(19) United States
(12) Patent Application Publication
(10) Pub. No.: US 2024/0252681 A1
(43) Pub. Date: Aug. 1, 2024

(54) GENE THERAPY CONSTRUCTS AND METHODS FOR TREATMENT OF HEARING LOSS

(71) Applicant: Rescue Hearing Inc., Gainesville, FL (US)

(72) Inventor: Hinrich Staecker, Leawood, KS (US)

(73) Assignee: Rescue Hearing Inc., Gainesville, FL (US)

(21) Appl. No.: 18/560,867

(22) PCT Filed: May 14, 2022
(86) PCT No.: PCT/US2022/029334

§ 371 Date: Nov. 14, 2023

Related U.S. Application Data
(60) Provisional application No. 63/188,857, filed on May 14, 2021.

(51) Int. Cl. A61K 48/00; A01K 67/0278; A61K 9/00; A61K 38/17; A61P 27/02; C12N 15/86

(52) U.S. Cl. CPC A61K 48/005; A01K 67/0278; A61K 9/0019; A61K 38/1709; A61K 48/0075; A61P 27/02; C12N 15/86; A01K 2217/072; A01K 2227/105; A01K 2267/0306; C12N 2740/15032; C12N 2740/15043

(57) ABSTRACT

Disclosed are compositions and methods that may be useful in the treatment and/or prevention of hearing loss caused by genetic mutation of the STRC gene. The compositions and methods disclosed herein use Lentiviral vectors to facilitate delivery of STRC into the inner ear to restore activity of the STRC gene, respectively, promote hair cell survival, prevent further degradation of hearing and/or restore hearing in patients suffering from hearing loss.

### CROSS-REFERENCE TO RELATED APPLICATIONS

The present application claims the benefit of and priority to U.S. Provisional Application No. 63/188,857, filed May 14, 2021, which is hereby incorporated by reference in its entirety for all purposes.

### TECHNICAL FIELD

The present disclosure provides compositions and methods useful in treating and/or preventing hearing loss. More particularly, the present disclosure provides compositions and methods useful for treating and/or preventing hearing loss caused by genetic mutation of the STRC gene.

### SEQUENCE LISTING

The instant application contains a Sequence Listing which has been filed electronically in ASCII format and is hereby incorporated by reference in its entirety. Said ASCII copy, created on May 14, 2022, is named BN00002_0051_SL_ST25.txt and is 56 KB in size.

### BACKGROUND

Hearing loss is the most common sensory deficit in humans. According to 2018 estimates on the magnitude of disabling hearing loss released by the World Health Organization (WHO), there are 466 million persons worldwide living with disabling hearing loss (432 million adults and 34 million children). The number of people with disabling hearing loss will grow to 630 million by 2030 and to over 900 million by 2050. Over 90% of persons with disabling hearing loss (420 million) reside in the low-income regions of the world (WHO global estimates on prevalence of hearing loss, Prevention of Deafness WHO 2018).

Research has demonstrated that more than 50% of prelingual deafness is genetic. Such hereditary hearing loss and deafness may be conductive, sensorineural, or a combination of both; syndromic (associated with malformations of the external ear or other organs or with medical problems involving other organ systems) or nonsyndromic (no associated visible abnormalities of the external ear or any related medical problems); and prelingual (before language develops) or postlingual (after language develops). Additionally, research has shown that more than 70% of hereditary hearing loss is nonsyndromic. The different gene loci for nonsyndromic deafness are designated DFN (for DeaFNess). Loci are named based on mode of inheritance: DFNA (Autosomal dominant), DFNB (Autosomal recessive) and DFNX (X-linked). The number following the above designations reflects the order of gene mapping and/or discovery. In the general population, the prevalence of hearing loss increases with age. This change reflects the impact of genetics and environment and the interactions between environmental triggers and an individual's genetic predisposition.

The current treatment options for those with disabling hearing loss are hearing aids or cochlear implants. Cochlear implantation is a common procedure with a large associated healthcare cost, over $1,000,000 lifetime cost per patient. The lifetime cost of cochlear implants and hearing aids is prohibitive for most people, and particularly for those living in low-income regions (where the majority of persons with disabling hearing loss reside). Unfortunately, there are currently no approved therapeutic agents for preventing or treating hearing loss or deafness. Accordingly, there is an urgent need for therapeutic options to provide cost effective alternatives to cochlear implants and hearing aids for hearing loss.

### SUMMARY

The present disclosure is based, at least in part, on the discovery that full length or near full length Stereocilin (STRC) may be incorporated into a lentivirus vector under the control of an inner ear specific promoter (e.g., a mouse or human Myo7A promoter) to generate robust expression of STRC in inner ear cells that is able to rescue the phenotypes associated with STRC loss-of-function mutations. The techniques herein provide the ability to rescue STRC loss-of-function mutations in mammals (e.g., humans) via gene therapy. The disclosure provides compositions and methods for restoring STRC function to patients suffering from disorders that result from STRC mutations.

In an aspect, the disclosure provides a lentivirus expression vector that includes a nucleic acid sequence encoding Stereocilin (STRC), or a part thereof, and a promoter operatively linked to the nucleic acid sequence.

In embodiments, the lentivirus expression vector is a third-generation self-inactivating (SIN) lentivirus vector. In embodiments, the SIN lentivirus vector lacks wildtype lentivirus long-terminal repeat (LTR) enhancer and promoter elements.

In embodiments, the promoter is selected from the group consisting of STRC promoters, Myo7a promoters, human cytomegalovirus (HCMV) promoters, cytomegalovirus/chicken beta-actin (CBA) promoters and Pou4f3 promoters. In embodiments, the promoter is Myo7a. In embodiments, the promoter is 95%, 96%, 97%, 98%, 99%, or 100% identical to SEQ ID NO: 4 or SEQ ID NO: 6. Optionally, the Myo7a promoter further includes a Myo7a enhancer. Optionally, the Myo7a promoter further includes a Myo7a enhancer. In embodiments where the promoter is 95%, 96%, 97%, 98%, 99%, or 100% identical to SEQ ID NO: 4 or SEQ ID NO: 6, the promoter may optionally further include a Myo7a enhancer 95%, 96%, 97%, 98%, 99%, or 100% identical to SEQ ID NO: 5.

In embodiments, the nucleic acid is 95%, 96%, 97%, 98%, 99%, or 100% identical to SEQ ID NO: 1. In embodiments, the nucleic acid encodes a polypeptide 95%, 96%, 97%, 98%, 99%, or 100% identical to SEQ ID NO: 2.

In an aspect, the disclosure provides a pharmaceutical composition for use in a method for the treatment or prevention of hearing loss comprising a lentivirus expression vector comprising a nucleic acid which is 95%, 96%, 97%, 98%, 99%, or 100% identical to SEQ ID NO: 1, wherein the nucleic acid sequence is operatively linked to a nucleic acid which is 95%, 96%, 97%, 98%, 99%, or 100% identical to SEQ ID NO: 4 or SEQ ID NO: 6.

In an aspect, the disclosure provides a cell comprising a lentivirus expression vector comprising the nucleic acid sequence of SEQ ID NO:1; and a promoter operatively linked to the nucleic acid.

In embodiments, the nucleic acid which is 95%, 96%, 97%, 98%, 99%, or 100% identical to SEQ ID NO: 1.

In embodiments, the promoter is selected from the group consisting of STRC promoters, Myo7a promoters, human cytomegalovirus (HCMV) promoters, cytomegalovirus/chicken beta-actin (CBA) promoters or Pou4f3 promoters.

In embodiments, the promoter is Myo7a. In embodiments, the promoter is 95%, 96%, 97%, 98%, 99%, or 100% identical to SEQ ID NO: 4 or SEQ ID NO: 6.

In embodiments, the cell is a stem cell. In embodiments, the stem cell is an induced pluripotent stem cell.

In an aspect, the disclosure provides a method for treating or preventing hearing loss including the step of: administering to a subject in need thereof an effective amount of the lentivirus vector of claim 1.

In embodiments, the promoter is selected from the group consisting of STRC promoters, Myo7a promoters, human cytomegalovirus (HCMV) promoters, cytomegalovirus/chicken beta-actin (CBA) promoters, or Pou4f3 promoters. In embodiments, the promoter is Myo7a. In embodiments, the promoter is 95%, 96%, 97%, 98%, 99%, or 100% identical to SEQ ID NO: 4 or SEQ ID NO: 6.

In embodiments, the expression vector is administered by injection into the inner ear of the subject.

In embodiments, the injection method is selected from the group consisting of cochleostomy, round window membrane, endolymphatic sac, scala media, canalostomy, scala media via the endolymphatic sac, or any combination thereof.

In embodiments, the subject has one or more genetic risk factors associated with hearing loss.

In embodiments, one of the genetic risk factors is selected from the group consisting of a mutation in the STRC gene.

In embodiments, the subject does not exhibit any clinical indicators of hearing loss.

In an aspect, the disclosure provides a transgenic mouse comprising a mutation/variation that causes hearing loss selected from a group consisting of a mutation/variation in the human STRC gene.

Disclosed herein is an expression vector including the nucleic acid sequence of SEQ ID NO:1 or SEQ ID NO:2, or a nucleic acid sequence having at least 90% sequence identity to the nucleic acid of SEQ ID NO:1 or SEQ ID NO:2, wherein the nucleic acid sequence is operatively linked to a promoter. Also disclosed herein is a pharmaceutical composition for use in a method for the treatment or prevention of hearing loss that includes an expression vector having the nucleic acid sequence of SEQ ID NO:1 or SEQ ID NO:2, or a nucleic acid sequence having at least 90% sequence identity to the nucleic acid of SEQ ID NO:1 or SEQ ID NO:2, wherein the nucleic acid sequence is operatively linked to a promoter. In some embodiments, the nucleic acid sequence has at least 75%, at least 80%, at least 85%, at least 90%, at least 95%, at least 96%, at least 97%, at least 98%, or at least 99% sequence identity to the nucleic acid sequence of SEQ ID NO:1 or SEQ ID NO:2. In some embodiments, the expression vector is selected from a lentiviral vector, an adeno-associated viral vector, an adenoviral vector, a herpes simplex viral vector, a vaccinia viral vector, or a helper dependent adenoviral vector. In some embodiments, the vector is a lentiviral vector or an adeno-associated viral vector selected from AAV2, AAV2/Anc80, AAV5, AAV6, AAV6.2, AAV7, AAV8, AAV9, AAVrh8, AAVrh10, AAVrh39, AAVrh43AAV1, AAV2, AAV3, AAV4, AAV5, AAV6, AAV7, AAV8 or Anc80. In some embodiments, the AAV vector may be an AAV50 mixed capsid, which has been shown to yield better transfection of inner and outer hair cells in adult animals when compared to Anc80. In some embodiments, the promoter is selected from any hair cell promoter that drives the expression of an operably linked nucleic acid at early development and maintains expression throughout the life, for example, STRC promoters, human cytomegalovirus (HCMV) promoters, cytomegalovirus/chicken beta-actin (CBA) promoters, Myo7a promoters or Pou4f3 promoters. In some embodiments, the enhancer may be the Barhl1 enhancer (see e.g., Hou et al. (2019) Cell 8(5):458). Examples of endogenous STRC promoters and enhancers are shown in Table 1.

TABLE 1

| | | TSS | # of | | Transcription | |
|---|---|---|---|---|---|---|
| GeneHancer ID | Type | distance (kb) | genes away | Size (kb) | Factor Binding Sites | Genomic location |
| GH15J043710 | Enhancer | +0.5 | 0 | 0.7 | — | chr15: 43710601- |
| | | | | | | 43711276 |
| | | | | | | (GRCh38/hg38) |
| | | | | | | chr15: 44002799- |
| | | | | | | 44003474 |
| | | | | | | (GRCh37/hg19) |
| GH15J043791 | Promoter/Enhancer | −85.0 | 19 | 11.0 | 236 | chr15: 43791000- |
| | | | | | | 43802001 |
| | | | | | | (GRCh38/hg38) |
| | | | | | | chr15: 44083198- |
| | | | | | | 44094199 |
| | | | | | | (GRCh37/hg19) |
| GH15J043745 | Promoter/Enhancer | −36.6 | 9 | 6.1 | 120 | chr15: 43745001- |
| | | | | | | 43751070 |
| | | | | | | (GRCh38/hg38) |
| | | | | | | chr15: 44037199- |
| | | | | | | 44043268 |
| | | | | | | (GRCh37/hg19) |
| GH15J043823 | Promoter/Enhancer | −114.6 | 24 | 5.8 | 237 | chr15: 43823173- |
| | | | | | | 43829001 |
| | | | | | | (GRCh38/hg38) |
| | | | | | | chr15: 44115371- |
| | | | | | | 44121199 |
| | | | | | | (GRCh37/hg19) |
| GH15J043774 | Promoter/Enhancer | −65.1 | 11 | 3.7 | 150 | chr15: 43774681- |
| | | | | | | 43778401 |
| | | | | | | (GRCh38/hg38) |
| | | | | | | chr15: 44066879- |
| | | | | | | 44070599 |
| | | | | | | (GRCh37/hg19) |
| GH15J044440 | Enhancer | −728.1 | 83 | 8.8 | 31 | chr15: 44435202- |
| | | | | | | 44444000 |
| | | | | | | (GRCh38/hg38) |
| | | | | | | chr15: 44727400- |
| | | | | | | 44736198 |
| | | | | | | (GRCh37/hg19) |
| GH15J043819 | Enhancer | −108.0 | 23 | 1.0 | 1 | chr15: 43819001- |
| | | | | | | 43820000 |
| | | | | | | (GRCh38/hg38) |
| | | | | | | chr15: 44111199- |
| | | | | | | 44112198 |
| | | | | | | (GRCh37/hg19) |
| GH15J043754 | Enhancer | −42.8 | 9 | 2.2 | — | chr15: 43753201- |
| | | | | | | 43755400 |
| | | | | | | (GRCh38/hg38) |
| | | | | | | chr15: 44045399- |
| | | | | | | 44047598 |
| | | | | | | (GRCh37/hg19) |
| GH15J043521 | Enhancer | +188.8 | 33 | 1.9 | — | chr15: 43521799- |
| | | | | | | 43523663 |
| | | | | | | (GRCh38/hg38) |
| | | | | | | chr15: 43813997- |
| | | | | | | 43815861 |
| | | | | | | (GRCh37/hg19) |
| GH15J043520 | Enhancer | +191.1 | 33 | 0.6 | — | chr15: 43520096- |
| | | | | | | 43520720 |
| | | | | | | (GRCh38/hg38) |
| | | | | | | chr15: 43812294- |
| | | | | | | 43812918 |
| | | | | | | (GRCh37/hg19) |
| GH15J043692 | Promoter | +17.7 | 4 | 2.4 | — | chr15: 43692600- |
| | | | | | | 43695000 |
| | | | | | | (GRCh38/hg38) |
| | | | | | | chr15: 43984798- |
| | | | | | | 43987198 |
| | | | | | | (GRCh37/hg19) |
| GH15J043648 | Promoter | +62.0 | 15 | 2.2 | — | chr15: 43648401- |
| | | | | | | 43650601 |
| | | | | | | (GRCh38/hg38) |
| | | | | | | chr15: 43940599- |
| | | | | | | 43942799 |
| | | | | | | (GRCh37/hg19) |
| GH15J043646 | Enhancer | +64.9 | 16 | 0.4 | 1 | chr15: 43646401- |
| | | | | | | 43646800 |
| | | | | | | (GRCh38/hg38) |
| | | | | | | chr15: 43938599- |
| | | | | | | 43938998 |
| | | | | | | (GRCh37/hg19) |
| GH15J043662 | Enhancer | +48.3 | 10 | 0.4 | — | chr15: 43663001- |
| | | | | | | 43663399 |
| | | | | | | (GRCh38/hg38) |
| | | | | | | chr15: 43955199- |
| | | | | | | 43955597 |
| | | | | | | (GRCh37/hg19) |
| GH15J043666 | Enhancer | +45.3 | 10 | 0.8 | — | chr15: 43665801- |
| | | | | | | 43666600 |
| | | | | | | (GRCh38/hg38) |
| | | | | | | chr15: 43957999- |
| | | | | | | 43958798 |
| | | | | | | (GRCh37/hg19) |
| GH15J043622 | Enhancer | +88.9 | 21 | 0.4 | — | chr15: 43622400- |
| | | | | | | 43622801 |
| | | | | | | (GRCh38/hg38) |
| | | | | | | chr15: 43914598- |
| | | | | | | 43914999 |
| | | | | | | (GRCh37/hg19) |
| GH15J043612 | Enhancer | +98.5 | 24 | 0.4 | — | chr15: 43612801- |
| | | | | | | 43613200 |
| | | | | | | (GRCh38/hg38) |
| | | | | | | chr15: 43904999- |
| | | | | | | 43905398 |
| | | | | | | (GRCh37/hg19) |
| GH15J043610 | Enhancer | +100.4 | 24 | 0.6 | — | chr15: 43610801- |
| | | | | | | 43611400 |
| | | | | | | (GRCh38/hg38) |
| | | | | | | chr15: 43902999- |
| | | | | | | 43903598 |
| | | | | | | (GRCh37/hg19) |

Disclosed herein is a cell having an expression vector that includes the nucleic acid sequence of SEQ ID NO:1, or a nucleic acid sequence having at least 90% sequence identity to the nucleic acid of SEQ ID NO:1, wherein the nucleic acid sequence is operatively linked to a promoter. In some embodiments, the nucleic acid sequence has at least 75%, at least 80%, at least 85%, at least 90%, at least 95%, at least 96%, at least 97%, at least 98%, at least 99%, or at least 100% sequence identity to the nucleic acid sequence of SEQ ID NO:1. In some embodiments, the cell is a stem cell. In some embodiments, the stem cell is an induced pluripotent stem cell.

Disclosed herein is a method for treating or preventing hearing loss, including administering to a subject in need thereof an effective amount of an expression vector that includes the nucleic acid sequence of SEQ ID NO:1, or a nucleic acid sequence having at least 90% sequence identity to the nucleic acid of SEQ ID NO:1, wherein the nucleic acid sequence is operatively linked to a promoter. In some embodiments, the nucleic acid sequence has at least 75%, at least 80%, at least 85%, at least 90%, at least 95%, at least 96%, at least 97%, at least 98%, or at least 99% sequence identity to the nucleic acid sequence of SEQ ID NO:1. In some embodiments, the expression vector is selected from a lentiviral vector, an adeno-associated viral vector, an adenoviral vector, a herpes simplex viral vector, a vaccinia viral vector, a helper dependent adenoviral vector. In some embodiments, the vector is a lentiviral vector or an adeno-associated viral vector selected from AAV2, AAV2/Anc80, AAV5, AAV6, AAV6.2, AAV7, AAV8, AAV9, AAVrh8, AAVrh10, AAVrh39, AAVrh43, AAV1, AAV2, AAV3, AAV4, AAV5, AAV6, AAV7, AAV8, Anc80, or AAV50. In some embodiments, the promoter is selected from any hair cell promoter that drives the expression of an operably linked nucleic acid sequence at early development and maintains expression throughout the life, for example, STRC promoters, human cytomegalovirus (HCMV) promoters, cytomegalovirus/chicken beta-actin (CBA) promoters, Myo7a promoters or Pou4f3 promoters. In some embodiments, the expression vector is administered into the inner ear of the subject, for example, by injection. In some embodiments, the delivery method is selected from cochleostomy, round window membrane, canalostomy or any combination thereof (see e.g., Erin E. Leary Swan, et al., Inner Ear Drug Delivery for Auditory Applications; Adv Drug Deliv Rev. 2008 December 14; 60(15): 1583-1599). In some embodiments, the expression vector is delivered into the scala media via the endolymphatic sac (see e.g., Colletti V, et al., Evidence of gadolinium distribution from the endolymphatic sac to the endolymphatic compartments of the human inner ear, Audiol Neurootol, 2010; 15(6):353-63; Marco Mandalà, MD, et al., Induced endolymphatic flow from the endolymphatic sac to the cochlea in Ménière's disease, Otolaryngology-Head and Neck Surgery (2010) 143, 673-679; Yamasoba T, et al., Inner ear transgene expression after adenoviral vector inoculation in the endolymphatic sac, Hum Gene Ther. 1999 Mar. 20; 10(5):769-74). In some embodiments, the subject has one or more genetic risk factors associated with hearing loss. In some embodiments, one of the genetic risk factors is a mutation in the STRC gene. In some embodiments, the mutation in the STRC gene is selected from any one or more STRC mutations known to cause hearing loss (see e.g., Table 4). In some embodiments, the subject does not exhibit any clinical indicators of hearing loss.

In some embodiments, an expression vector described herein is administered as a combination therapy with one or more expression vectors comprising other nucleic acid sequences and/or with one or more other active pharmaceutical agents for treating hearing loss. For example, a combination therapy may include a first expression vector that has the nucleic acid sequence of SEQ ID NO:1 and a second expression vector that has a nucleic acid sequence, wherein both expression vectors are administered to a subject as part of a combination therapy to treat hearing loss.

Disclosed herein is a transgenic mouse having a human STRC gene with a mutation selected from any one or more STRC mutation known to cause hearing loss (see, for example, Table 4).

### Definitions

By “alteration” is meant an increase or decrease. An alteration may be by as little as 1%, 2%, 3%, 4%, 5%, l0%, 20%, 30%, or by 40%, 50%, 60%, or even by as much as 75%, 80%, 90%, or 100%.

By “biologic sample” is meant any tissue, cell, fluid, or other material derived from an organism.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 70%, more preferably 80% or 85%, and more preferably 90%, 91%, 92%, 93%, 94%, 95%, 96%, 97%, 98%, or even 99% identical at the amino acid level or nucleic acid to the sequence used for comparison.

By “fusion protein” is meant an engineered polypeptide that combines sequence elements excerpted from two or more other proteins.

As used herein, the terms “transfect,” “transfects,” “transfecting” and “transfection” refer to the delivery of nucleic acids (usually DNA or RNA) to the cytoplasm or nucleus of cells, e.g., through the use of cationic lipid vehicle(s) and/or by means of electroporation, or other art-recognized means of transfection.

By “transduction,” is meant the delivery of nucleic acids (usually DNA or RNA) to the cytoplasm or nucleus of cells through the use of viral delivery, e.g., via lentiviral delivery vectors/plasmids, or other art-recognized means of transduction.

The term “plasmid” as used herein refers to an engineered construct comprised of genetic material designed to direct transformation of a targeted cell. The plasmid consists of a plasmid backbone. A “plasmid backbone” as used herein contains multiple genetic elements positional and sequentially oriented with other necessary genetic elements such that the nucleic acid in a nucleic acid cassette can be transcribed and when necessary translated in the transfected or transduced cells. The term plasmid as used herein can refer to nucleic acid, e.g., DNA derived from a plasmid vector, cosmid, phagemid or bacteriophage, into which one or more fragments of nucleic acid may be inserted or cloned which encode for particular genes

A “viral vector” as used herein is one that is physically incorporated in a viral particle by the inclusion of a portion of a viral genome within the vector, e.g., a packaging signal, and is not merely DNA or a located gene taken from a portion of a viral nucleic acid. Thus, while a portion of a viral genome can be present in a plasmid of the present disclosure, that portion does not cause incorporation of the plasmid into a viral particle and thus is unable to produce an infective viral particle.

As used herein, the term “vector” refers to any genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors.

As used herein, the term “integrating vector” refers to a vector whose integration or insertion into a nucleic acid (e.g., a chromosome) is accomplished via an integrase. Examples of “integrating vectors” include, but are not limited to, retroviral vectors, transposons, and adeno associated virus vectors.

As used herein, the term “integrated” refers to a vector that is stably inserted into the genome (i.e., into a chromosome) of a host cell.

As used herein, the term “exogenous gene” refers to a gene that is not naturally present in a host organism or cell, or is artificially introduced into a host organism or cell.

The term “gene” refers to a nucleic acid (e.g., DNA or RNA) sequence that comprises coding sequences necessary for the production of a precursor or polypeptide (e.g., STRC). The polypeptide can be encoded by a full-length coding sequence or by any portion of the coding sequence so long as the desired activity or functional properties (e.g., improved hair cell survival and hair cell function) of the full-length or fragment are retained. The term also encompasses the coding region of a structural gene and includes sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ untranslated sequences. The sequences that are located 3′ or downstream of the coding region and which are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

As used herein, the terms “nucleic acid molecule encoding,” “DNA sequence encoding,” “DNA encoding,” “RNA sequence encoding,” and “RNA encoding” refer to the order or sequence of deoxyribonucleotides or ribonucleotides along a strand of deoxyribonucleic acid or ribonucleic acid. The order of these deoxyribonucleotides or ribonucleotides determines the order of amino acids along the polypeptide (protein) chain. The DNA or RNA sequence thus codes for the amino acid sequence.

As used herein, the term “variant,” when used in reference to a protein, refers to proteins encoded by partially homologous nucleic acids so that the amino acid sequence of the proteins varies. As used herein, the term “variant” encompasses proteins encoded by homologous genes having both conservative and nonconservative amino acid substitutions that do not result in a change in protein function, as well as proteins encoded by homologous genes having amino acid substitutions that cause decreased (e.g., null mutations) protein function or increased protein function.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

As used herein, the term “regulatory element” refers to a genetic element which controls some aspect of the expression of nucleic acid sequences. For example, a promoter is a regulatory element that facilitates the initiation of transcription of an operably linked coding region. Other regulatory elements are splicing signals, polyadenylation signals, termination signals, RNA export elements, internal ribosome entry sites, etc.

Transcriptional control signals in eukaryotes comprise “promoter” and “enhancer” elements. Promoters and enhancers consist of short arrays of DNA sequences that interact specifically with cellular proteins involved in transcription (Maniatis et al., (1987) Science 236:1237). Promoter and enhancer elements have been isolated from a variety of eukaryotic sources including genes in yeast, insect and mammalian cells, and viruses (analogous control elements, i.e., promoters, are also found in prokaryotes). The selection of a particular promoter and enhancer depends on what cell type is to be used to express the protein of interest. Some eukaryotic promoters and enhancers have a broad host range while others are functional in a limited subset of cell types (for review see, Voss et al., (1986) Trends Biochem. Sci., 11:287; and Maniatis et al., supra). For example, the SV40 early gene enhancer is very active in a wide variety of cell types from many mammalian species and has been widely used for the expression of proteins in mammalian cells (Dijkema et al, (1985) EMBO J. 4:761). Two other examples of promoter/enhancer elements active in a broad range of mammalian cell types are those from the human elongation factor 1α gene (Uetsuki et al., (1989) J. Biol. Chem., 264:5791; Kim et al., (1990) Gene 91:217; and Mizushima and Nagata, (1990) Nuc. Acids. Res., 18:5322) and the long terminal repeats of the Rous sarcoma virus (Gorman et al., (1982) Proc. Natl. Acad. Sci. USA 79:6777) and the human cytomegalovirus (Boshart et al., (1985) Cell 41:521).

As used herein, the term “promoter/enhancer” denotes a segment of DNA which contains sequences capable of providing both promoter and enhancer functions (i.e., the functions provided by a promoter element and an enhancer element, see above for a discussion of these functions). For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques such as cloning and recombination) such that transcription of that gene is directed by the linked enhancer/promoter.

The term “promoter,” “promoter element,” or “promoter sequence” as used herein, refers to a DNA sequence which when ligated to a nucleotide sequence of interest is capable of controlling the transcription of the nucleotide sequence of interest into mRNA. A promoter is typically, though not necessarily, located 5′ (i.e., upstream) of a nucleotide sequence of interest whose transcription into mRNA it controls, and provides a site for specific binding by RNA polymerase and other transcription factors for initiation of transcription.

Promoters may be constitutive or regulatable. The term “constitutive” when made in reference to a promoter means that the promoter is capable of directing transcription of an operably linked nucleic acid sequence in the absence of a stimulus (e.g., heat shock, chemicals, etc.). In contrast, a “regulatable” promoter is one which is capable of directing a level of transcription of an operably linked nucleic acid sequence in the presence of a stimulus (e.g., heat shock, chemicals, etc.) which is different from the level of transcription of the operably linked nucleic acid sequence in the absence of the stimulus. Certain promoters are also known in the art to impart tissue-specificity and/or temporal/developmental specificity to expression of a nucleic acid sequence under control of such a promoter.

As used herein, the term “retrovirus” refers to a retroviral particle which is capable of entering a cell (i.e., the particle contains a membrane-associated protein such as an envelope protein or a viral G glycoprotein which can bind to the host cell surface and facilitate entry of the viral particle into the cytoplasm of the host cell) and integrating the retroviral genome (as a double-stranded provirus) into the genome of the host cell. The term “retrovirus” encompasses Oncovirinae (e.g., Moloney murine leukemia virus (Mo-LV, also recited as simply “MLV” herein), Moloney murine sarcoma virus (MoMSV), and Mouse mammary tumor virus (MMTV), Spumavirinae, and Lentivirinae (e.g., Human immunodeficiency virus, Simian immunodeficiency virus, Equine infection anemia virus, and Caprine arthritis-encephalitis virus; See, e.g., U.S. Pat. Nos. 5,994,136 and 6,013,516, both of which are incorporated herein by reference).

As used herein, the term “retroviral vector” refers to a retrovirus that has been modified to express a gene of interest. Retroviral vectors can be used to transfer genes efficiently into host cells by exploiting the viral infectious process. Foreign or heterologous genes cloned (i.e., inserted using molecular biological techniques) into the retroviral genome can be delivered efficiently to host cells which are susceptible to infection by the retrovirus.

As used herein, the term “lentivirus vector” refers to retroviral vectors derived from the Lentiviridae family (e.g., human immunodeficiency virus, simian immunodeficiency virus, equine infectious anemia virus, and caprine arthritis-encephalitis virus) that are capable of integrating into non-dividing cells (See, e.g., U.S. Pat. Nos. 5,994,136 and 6,013,516, both of which are incorporated herein by reference).

As used herein, the term “adeno-associated virus (AAV) vector” refers to a vector derived from an adeno-associated virus serotype, including without limitation, AAV-1, AAV-2, AAV-3, AAV-4, AAV-5, AAV-6, AAV-7, AAV-8, AAV-9, etc. AAV vectors can have one or more of the AAV wild-type genes deleted in whole or part, preferably the rep and/or cap genes, but retain functional flanking ITR sequences.

As used herein the term, the term “in vitro” refers to an artificial environment and to processes or reactions that occur within an artificial environment. In vitro environments can consist of, but are not limited to, test tubes and cell cultures. The term “in vivo” refers to the natural environment (e.g., an animal or a cell) and to processes or reaction that occur within a natural environment.

As used herein, the term “host cell” refers to any eukaryotic cell (e.g., mammalian cells, avian cells, amphibian cells, plant cells, fish cells, and insect cells), whether located in vitro or in vivo.

The term “administration” refers to introducing a substance into a subject. In general, any route of administration may be utilized including, for example, parenteral (e.g., intravenous), oral, topical, subcutaneous, peritoneal, intra-arterial, inhalation, vaginal, rectal, nasal, introduction into the cerebrospinal fluid, or instillation into body compartments. In some embodiments, administration is oral. Additionally or alternatively, in some embodiments, administration is parenteral. In some embodiments, administration is intravenous.

By “agent” is meant any small compound (e.g., small molecule), antibody, nucleic acid molecule, or polypeptide, or fragments thereof or cellular therapeutics such as allogeneic transplantation and/or CART-cell therapy.

By “STRC nucleic acid molecule” is meant a polynucleotide that encodes a STRC polypeptide. An exemplary STRC nucleic acid molecule is 95%, 96%, 97%, 98%, 99%, or 100% identical to the following sequence (e.g., NM_153700)(SEQ ID NO:1):

>NM_153700
gccctgccctcacctggctatcccacacaggtgagaataaccagaactcacctccggtac
cagtgttcacttggaaacatggctctcagcctctggcccctgctgctgctgctgctgctg
ctgctgctgctgtcctttgcagtgactctggcccctactgggcctcattccctggaccct
ggtctctccttcctgaagtcattgctctccactctggaccaggctccccagggctccctg
agccgctcacggttctttacattcctggccaacatttcttcttcctttgagcctgggaga
atgggggaaggaccagtaggagagcccccacctctccagccgcctgctctgcggctccat
gattttctagtgacactgagaggtagccccgactgggagccaatgctagggctgctaggg
gatatgctggcactgctgggacaggagcagactccccgagatttcctggtgcaccaggca
ggggtgctgggtggacttgtggaggtgctgctgggagccttagttcctgggggcccccct
accccaactcggcccccatgcacccgtgatgggccgtctgactgtgtcctggctgctgac
tggttgccttctctgctgctgttgttagagggcacacgctggcaagctctggtgcaggtg
cagcccagtgtggaccccaccaatgccacaggcctcgatgggagggaggcagctcctcac
tttttgcagggtctgttgggtttgcttaccccaacaggggagctaggctccaaggaggct
ctttggggcggtctgctacgcacagtgggggcccccctctatgctgcctttcaggagggg
ctgctccgtgtcactcactccctgcaggatgaggtcttctccattttggggcagccagag
cctgataccaatgggcagtgccagggaggtaaccttcaacagctgctcttatggggcgtc
cggcacaacctttcctgggatgtccaggcgctgggctttctgtctggatcaccaccccca
ccccctgccctccttcactgcctgagcacgggcgtgcctctgcccagagcttctcagccg
tcagcccacatcagcccacgccaacggcgagccatcactgtggaggccctctgtgagaac
cacttaggcccagcaccaccctacagcatttccaacttctccatccacttgctctgccag
cacaccaagcctgccactccacagccccatcccagcaccactgccatctgccagacagct
gtgtggtatgcagtgtcctgggcaccaggtgcccaaggctggctacaggcctgccacgac
cagtttcctgatgagtttttggatgcgatctgcagtaacctctccttttcagccctgtct
ggctccaaccgccgcctggtgaagcggctctgtgctggcctgctcccaccccctaccagc
tgccctgaaggcctgccccctgttcccctcaccccagacatcttttggggctgcttcttg
gagaatgagactctgtgggctgagcgactgtgtggggaggcaagtctacaggctgtgccc
cccagcaaccaggcttgggtccagcatgtgtgccagggccccaccccagatgtcactgcc
tccccaccatgccacattggaccctgtggggaacgctgcccggatgggggcagcttcctg
gtgatggtctgtgccaatgacaccatgtatgaggtcctggtgcccttctggccttggcta
gcaggccaatgcaggataagtcgtgggggcaatgacacttgcttcctagaagggctgctg
ggcccccttctgccctctctgccaccactgggaccatccccactctgtctgacccctggc
cccttcctccttggcatgctatcccagttgccacgctgtcagtcctctgtcccagctctt
gctcaccccacacgcctacactatctcctccgcctgctgaccttcctcttgggtccaggg
gctgggggcgctgaggcccaggggatgctgggtcgggccctactgctctccagtctccca
gacaactgctccttctgggatgcctttcgcccagagggccggcgcagtgtgctacggacg
attggggaatacctggaacaagatgaggagcagccaaccccatcaggctttgaacccact
gtcaaccccagctctggtataagcaagatggagctgctggcctgctttagtcctgtgctg
tgggatctgctccagagggaaaagagtgtttgggccctgcagattctagtgcaggcgtac
ctgcatatgcccccagaaaacctccagcagctggtgctttcagcagagagggaggctgca
cagggcttcctgacactcatgctgcaggggaagctgcaggggaagctgcaggtaccacca
tccgaggagcaggccctgggtcgcctgacagccctgctgctccagcggtacccacgcctc
acctcccagctcttcattgacctgtcaccactcatccctttcttggctgtctctgacctg
atgcgcttcccaccatccctgttagccaacgacagtgtcctggctgccatccgggattac
agcccaggaatgaggcctgaacagaaggaggctctggcaaagcgactgctggcccctgaa
ctgtttggggaagtgcctgcctggccccaggagctgctgtgggcagtgctgcccctgctc
ccccacctccctctggagaactttttgcagctcagccctcaccagatccaggccctggag
gatagctggccagcagcaggtctggggccagggcatgcccgccatgtgctgcgcagcctg
gtaaaccagagtgtccaggatggtgaggagcaggtacgcaggcttgggcccctcgcctgt
ttcctgagccctgaggagctgcagagcctagtgcccctgagtgatccaacggggccagta
gaacgggggctgctggaatgtgcagccaatgggaccctcagcccagaaggacgggtggca
tatgaacttctgggtgtgttgcgctcatctggaggagcggtgctgagcccccgggagctg
cgggtctgggcccctctcttctctcagctgggcctccgcttccttcaggagctgtcagag
ccccagcttagagccatgcttcctgtcctgcagggaactagtgttacacctgctcaggct
gtcctgctgcttggacggctccttcctaggcacgatctatccctggaggaactctgctcc
ttgcaccttctgctaccaggcctcagcccccagacactccaggccatccctaggcgagtc
ctggtcggggcttgttcctgcctggcccctgaactgtcacgcctctcagcctgccagacc
gcagcactgctgcagacctttcgggttaaagatggtgttaaaaatatgggtacaacaggt
gctggtccagctgtgtgtatccctggtcagcctattcccaccacctggccagactgcctg
cttcccctgctcccattaaagctgctacaactggattccttggctcttctggcaaatcga
agacgctactgggagctgccctggtctgagcagcaggcacagtttctctggaagaagatg
caagtacccaccaaccttaccctcaggaatctgcaggctctgggcaccctggcaggaggc
atgtcctgtgagtttctgcagcagatcaactccatggtagacttccttgaagtggtgcac
atgatctatcagctgcccactagagttcgagggagcctgagggcctgtatctgggcagag
ctacagcggaggatggcaatgccagaaccagaatggacaactgtagggccagaactgaac
gggctggatagcaagctactcctggacttaccgatccagttgatggacagactatccaat
gaatccattatgttggtggtggagctggtgcaaagagctccagagcagctgctggcactg
acccccctccaccaggcagccctggcagagagggcactacaaaacctggctccaaaggag
actccagtctcaggggaagtgctggagaccttaggccctttggttggattcctggggaca
gagagcacacgacagatccccctacagatcctgctgtcccatctcagtcagctgcaaggc
ttctgcctaggagagacatttgccacagagctgggatggctgctattgcaggagtctgtt
cttgggaaaccagagttgtggagccaggatgaagtagagcaagctggacgcctagtattc
actctgtctactgaggcaatttccttgatccccagggaggccttgggtccagagaccctg
gagcggcttctagaaaagcagcagagctgggagcagagcagagttggacagctgtgtagg
gagccacagcttgctgccaagaaagcagccctggtagcaggggtggtgcgaccagctgct
gaggatcttccagaacctgtgccaaattgtgcagatgtacgagggacattcccagcagcc
tggtctgcaacccagattgcagagatggagctctcagactttgaggactgcctgacatta
tttgcaggagacccaggacttgggcctgaggaactgcgggcagccatgggcaaagcaaaa
cagttgtggggtcccccccggggatttcgtcctgagcagatcctgcagcttggtaggctc
ttaataggtctaggagatcgggaactacaggagctgatcctagtggactggggagtgctg
agcaccctggggcagatagatggctggagcaccactcagctccgcattgtggtctccagt
ttcctacggcagagtggtcggcatgtgagccacctggacttcgttcatctgacagcgctg
ggttatactctctgtggactgcggccagaggagctccagcacatcagcagttgggagttc
agccaagcagctctcttcctcggcaccctgcatctccagtgctctgaggaacaactggag
gttctggcccacctacttgtactgcctggtgggtttggcccaatcagtaactgggggcct
gagatcttcactgaaattggcaccatagcagctgggatcccagacctggctctttcagca
ctgctgcggggacagatccagggcgttactcctcttgccatttctgtcatccctcctcct
aaatttgctgtggtgtttagtcccatccaactatctagtctcaccagtgctcaggctgtg
gctgtcactcctgagcaaatggcctttctgagtcctgagcagcgacgagcagttgcatgg
gcccaacatgagggaaaggagagcccagaacagcaaggtcgaagtacagcctggggcctc
caggactggtcacgaccttcctggtccctggtattgactatcagcttccttggccacctg
ctatgagcctgtctctacagtagaaggagattgtggggagagaaatcttaagtcataatg
aataaagtgcaaacagaagtgcatcctgattattttcagaagctgatgaggaata

By “STRC polypeptide” is meant a polypeptide, or fragment thereof, having at least 95%, 96%, 97%, 98%, 99%, or 100% identical to the following sequence (e.g., NP_714544.1)(SEQ ID NO:2):

>NP_714544.1
MALSLWPLLLLLLLLLLLSFAVTLAPTGPHSLDPGLSFLKSLLSTLDQAPQGSLSRSRFF
TFLANISSSFEPGRMGEGPVGEPPPLQPPALRLHDFLVTLRGSPDWEPMLGLLGDMLALL
GQEQTPRDFLVHQAGVLGGLVEVLLGALVPGGPPTPTRPPCTRDGPSDCVLAADWLPSLL
LLLEGTRWQALVQVQPSVDPTNATGLDGREAAPHFLQGLLGLLTPTGELGSKEALWGGLL
RTVGAPLYAAFQEGLLRVTHSLQDEVFSILGQPEPDTNGQCQGGNLQQLLLWGVRHNLSW
DVQALGFLSGSPPPPPALLHCLSTGVPLPRASQPSAHISPRQRRAITVEALCENHLGPAP
PYSISNFSIHLLCQHTKPATPQPHPSTTAICQTAVWYAVSWAPGAQGWLQACHDQFPDEF
LDAICSNLSFSALSGSNRRLVKRLCAGLLPPPTSCPEGIPPVPLTPDIFWGCFLENETLW
AERLCGEASLQAVPPSNQAWVQHVCQGPTPDVTASPPCHIGPCGERCPDGGSFLVMVCAN
DTMYEVLVPFWPWLAGQCRISRGGNDTCFLEGLLGPLLPSLPPLGPSPLCLTPGPFLLGM
LSQLPRCQSSVPALAHPTRLHYLLRLLTFLLGPGAGGAEAQGMLGRALLLSSLPDNCSFW
DAFRPEGRRSVLRTIGEYLEQDEEQPTPSGFEPTVNPSSGISKMELLACFSPVLWDLLQR
EKSVWALQILVQAYLHMPPENLQQLVLSAEREAAQGFLTLMLQGKLQGKLQVPPSEEQAL
GRLTALLLQRYPRLTSQLFIDLSPLIPFLAVSDLMRFPPSLLANDSVLAAIRDYSPGMRP
EQKEALAKRLLAPELFGEVPAWPQELLWAVLPLLPHLPIENFLQLSPHQIQALEDSWPAA
GLGPGHARHVLRSLVNQSVQDGEEQVRRLGPLACFLSPEELQSLVPLSDPTGPVERGLLE
CAANGTLSPEGRVAYELLGVLRSSGGAVLSPRELRVWAPLFSQLGLRFLQELSEPQLRAM
LPVLQGTSVTPAQAVLLLGRLLPRHDLSLEELCSLHLLIPGLSPQTLQAIPRRVLVGACS
CLAPELSRLSACQTAALLQTFRVKDGVKNMGTTGAGPAVCIPGQPIPTTWPDCLLPLLPL
KLLQLDSLALLANRRRYWELPWSEQQAQFLWKKMQVPTNLTLRNLQALGTLAGGMSCEFL
QQINSMVDFLEVVHMIYQLPTRVRGSLRACIWAELQRRMAMPEPEWTTVGPELNGLDSKL
LLDLPIQLMDRLSNESIMLVVELVQRAPEQLLALTPLHQAALAERALQNLAPKETPVSGE
VLETLGPLVGFLGTESTRQIPLQILLSHLSQLQGFCLGETFATELGWLLLQESVLGKPEL
WSQDEVEQAGRLVFTLSTEAISLIPREALGPETLERLLEKQQSWEQSRVGQLCREPQLAA
KKAALVAGVVRPAAEDLPEPVPNCADVRGTFPAAWSATQIAEMELSDFEDCLTLFAGDPG
LGPEELRAAMGKAKQLWGPPRGFRPEQILQLGRLLIGLGDRELQELILVDWGVLSTLGQI
DGWSTTQLRIVVSSFLRQSGRHVSHLDFVHLTALGYTLCGLRPEELQHISSWEFSQAALF
LGTLHLQCSEEQLEVLAHLLVLPGGFGPISNWGPEIFTEIGTIAAGIPDLALSALLRGQI
QGVTPLAISVIPPPKFAVVFSPIQLSSLTSAQAVAVTPEQMAFLSPEQRRAVAWAQHEGK
ESPEQQGRSTAWGLQDWSRPSWSLVLTISFLGHLL

By “STRC genomic sequence” is meant a genomic polynucleotide that encodes a STRC polypeptide. An exemplary STRC genomic sequence is 95%, 96%, 970% 98%, 99%, or 100% identical to the following sequence (e.g., NC_000015.10)(SEQ ID NO:3):

>NC_000015.10
gccctgccctcacctggctatcccacacaggtgagaataaccagaactcacctccggtac
cagtgttcacttggaaacatggctctcagcctctggcccctgctgctgctgctgctgctg
ctgctgctgctgtcctttgcaggtaagaagaacagtgagcagaactggggatgaggagga
gggtggctggaaaaagactttaagaatatggaggtgaacctgttagatagaaggacaaag
gagagaggcagagacttgtgcaaaagggaaaaatgagggttaagaaaagcaggccaagac
ttactgtaggccagtgaaaggggttcagctcaccatcccctcacctcatctttagatcca
ggtagggaactgtgctcaggggcagggttgagtttgggctctgtgttcctctccttcagt
gacctctggtttctctccttacagtgactctggcccctactgggcctcattccctggacc
ctggtctctccttcctgaagtcattgctctccactctggaccaggctccccagggctccc
tgagccgctcacggttctttacattcctggccaacatttcttcttcctttgagcctggga
gaatgggggaaggaccagtaggagagcccccacctctccagccgcctgctctgcggctcc
atgattttctagtgacactgagaggtagccccgactgggagccaatgctagggctgctag
gggatatgctggcactgctgggacaggagcagactccccgagatttcctggtgcaccagg
caggggtgctgggtggacttgtggaggtgctgctgggagccttagttcctgggggccccc
ctaccccaactcggcccccatgcacccgtgatgggccgtctgactgtgtcctggctgctg
actggttgccttctctgctgctgttgttagagggcacacgctggcaagctctggtgcagg
tgcagcccagtgtggaccccaccaatgccacaggcctcgatgggagggaggcagctcctc
actttttgcagggtctgttgggtttgcttaccccaacaggggagctaggctccaaggagg
ctctttggggcggtctgctacgcacagtgggggcccccctctatgctgcctttcaggagg
ggctgctccgtgtcactcactccctgcaggatgaggtcttctccattttggggcagccag
agcctgataccaatgggcagtgccagggaggtgagtgtggccagggctgggactgggatg
tggcagggcaaggaaagtgaaattggggtagttttcttccttactctttccctcctaggt
aaccttcaacagctgctcttatggtaagtaacaggagaccagttctgagggattgggcct
ggaaaatctggaggtgaagagctgaagacctcagcctctagagaggaaaactgatgggag
gagtgtagtttagtggttttggggtgtgactgtctgggttggtgtcccagctccacctct
tcctagccatatgaccttgagcaggttacatagtctttctatacctcagtttccccattt
ataaaatgagaatgataatattagttaccacagagttgttgcacccggttaaatgagttg
atactgtgtatgcaaacgacttaaaaccgtgctggcacatagcgcttaataatgttagct
agtaaagatgggatttggaaaataaggacacagctggattcctctacccccttactactt
cagtacaacaatgccagacagtagttagacatattgagttgctgagcagatttcctaaca
tgaggcccgctgagggttgtgtttaagctatctaaaagcatacgaagaaaggagacagaa
gggggccaggtggacagaaagaattccaactggggcttctcctaggtgattttggacctt
ggcagggcagctttctcttttttgccccgttgcagcatttcaaccagtaacgcctaaact
ctcagggacctcgcttgtagaaaagcctatgcttgccatgccccttgagggctctgagtc
agggtcagaatcttcagctggaggaaatgtgaactgaccagatcctgcctgctcctccct
ctgcacccaggggcgtccggcacaacctttcctgggatgtccaggcgctgggctttctgt
ctggatcaccacccccaccccctgccctccttcactgcctgagcacgggcgtgcctctgc
ccagagcttctcagccgtcagcccacatcagcccacgccaacggcgagccatcactgtgg
aggccctctgtgagaaccacttaggcccagcaccaccctacagcatttccaacttctcca
tccacttgctctgccagcacaccaagcctgccactccacagccccatcccagcaccactg
ccatctgccagacagctgtgtggtatgcagtgtcctgggcaccaggtgcccaaggctggc
tacaggcctgccacgaccagtttcctgatgagtttttggatgcgatctgcagtaacctct
ccttttcagccctgtctggctccaaccgccgcctggtgaagcggctctgtgctggcctgc
tcccaccccctaccagctgccctgaaggcctgccccctgttcccctcaccccagacatct
tttggggctgcttcttggagaatgagactctgtgggctgagcgactgtgtggggaggcaa
gtctacaggctgtgccccccagcaaccaggcttgggtccagcatgtgtgccagggcccca
ccccagatgtcactgcctccccaccatgccacattggaccctgtggggaacgctgcccgg
atgggggcagcttcctggtgatggtctgtgccaatgacaccatgtatgaggtcctggtgc
ccttctggccttggctagcaggccaatgcaggataagtcgtgggggcaatgacacttgct
tcctagaagggctgctgggcccccttctgccctctctgccaccactgggaccatccccac
tctgtctgacccctggccccttcctccttggcatgctatcccagttgccacgctgtcagt
cctctgtcccagctcttgctcaccccacacgcctacactatctcctccgcctgctgacct
tcctcttgggtccaggggctgggggcgctgaggcccaggggatgctgggtcgggccctac
tgctctccagtctcccagacaactgctccttctgggatgcctttcgcccagagggccggc
gcagtgtgctacggacgattggggaatacctggaacaagatgaggagcagccaaccccat
caggctttgaacccactgtcaaccccagctctggtataagcaagatggagctgctggcct
gctttagtgtgagtgctctgccagagggaaagctcctagaacagtgagaaggccctccag
gggaattcctcgaatactcagaggcagtagtgtggggtagtagttgaagcacacagctct
agagtcagacaggcttggattcatatcttggttctgtgaccagccttgaatgagttattt
aacttctctgagcaatatttttctcgtctcatttataaactagggatgataatggtatat
gagataatacatgctgtgggcttagcacagtgcatgatacacaaacatgcaataaatatt
accttgttattcttttgggctctttgactctctcactttctgcaccagaaagaaaaagga
tcaagttagaggactctaaatttttcccctagagagtgagaattggaggctggcagaata
caggaagataaggtaggaatgagaaagattcagggacactaccaatcagaagactttggt
tctaggttcaactgtgccacaaattagtgtgatcttaggcaagcaatttcatttagtttt
tctgggcttcagtttttagtctgtagaatggaggggtgagaatatgttaaacaccataat
taattcactgagtgcctattatatgcaaggcactttgctaggttctgtaggatatataaa
gatttcttactccatgttggggccacctttttcaaaccctgggcccagtaaaatggaatt
agatagtctcatagtatttggttcaggtctacaagtattaattgagccaactatggacct
ggcatgggagagggtacaagagaaattagagatatgatcccggacctaaaagagcttaat
atctgaagaatcacacttgagatgatggacaagcatcccagcaagtggagctggaatgcc
tgggggagctgcaggagagacagagaagacagctctgttggcatattgtctttcttccca
ccagcctgtgctgtgggatctgctccagagggaaaagagtgtttgggccctgcagattct
agtgcaggtaacaggtggagggcacatgggtgggctgggtgacagccatggctggaggtc
cctgccccgtgaggtgaggccatacccaccatgacctcctattcgcaggcgtacctgcat
atgcccccagaaaacctccagcagctggtgctttcagcagagagggaggctgcacagggc
ttcctgacactcatgctgcaggggaagctgcaggggaagctgcaggtgagcactgagaaa
ggggagcaagggcacctggagcctagtgttcagagggcttgctttagtgggaggaggaac
tccagagaggaaatggcagggatactgagcatctccagaggcagaatccattcctgtgcc
cctacaggtaccaccatccgaggagcaggccctgggtcgcctgacagccctgctgctcca
gcggtacccacgcctcacctcccagctcttcattgacctgtcaccactcatccctttctt
ggctgtctctgacctgatgcgcttcccaccatccctgttagccaacgacagtgtgtaagg
ttcttgcactactcctcctgctcctgtcacggtcaggccaaccgcatccacctggagcag
ccccttccggagctcctctctgtttttttctttcatgccagataggcaatgtgccaacat
cgtagcaaggtttgagagaggcacatctcacgcctgagtgtgaaaacccaatcattatgc
taatgaactacaaaaggatcagagagctcctctctattaaaaccagggagaggatgggcg
tggtggctcatgcctgtaatcccagcacgttgggagcccgaggcaggtggatcactaggt
ccgcctagtgagttcgagaccagcctggccaatatggtgaaaccccgtctctattaaact
acaaaaattagccaggcatggtggtgggcgcttgtagtcccagttactctggaggctgag
gcaggaggatagcttgaacctgggaggcagaggttgcagtgaaccaagatcgtgccactg
cactccagcctgggtgacagagcgagactccgtcttaaaaaaaacaaaaaacaaaacaaa
acaaaaaaacagggagagtctccttcctatctagacagcagggctacagagggtcagagg
aaaacagtttggaggaagacaaagggttaagacccatgactcctcgcagcctggctgcca
tccgggattacagcccaggaatgaggcctgaacagaaggaggctctggcaaagcgactgc
tggcccctgaactgtttggggaagtgcctgcctggccccaggagctgctgtgggcagtgc
tgcccctgctcccccacctccctctggagaactttttgcagctcagccctcaccaggtat
gagaatcatcttctttacttgactggcccatcttctgctagtggggacaaagagtcaatg
gcatgtctctcagtggcccctccctgcaagaaccctatagtgaccccagtgcgagctaac
cttccccatctcagatccaggccctggaggatagctggccagcagcaggtctggggccag
ggcatgcccgccatgtgctgcgcagcctggtaaaccagagtgtccaggatggtgaggagc
aggtacgcaggtgagttgttgtgggatcagtaaccaaggcaagagtggaagaggtagaga
gaggaaggcacagctgtcacgctgggtcggtgttctaggaagaaaggggcaagagagtag
gcagtggcctcaggcagcatagagttccaggagagaggtctatagatggtgcccctgtgt
agtggtgtagtgtcagagtgcccagtgtatgtacccataccatctgctgccaggcctgcc
ttagtgctagtcttggggaccacacaaaggtcagcttcatgccctcctcaggcttgggcc
cctcgcctgtttcctgagccctgaggagctgcagagcctagtgcccctgagtgatccaac
ggggccagtagaacgggggctgctggaatgtgcagccaatgggaccctcagcccagaagg
acgggtgagcccctcagcacaagcctacaagactttaggcttcccctgggtctgtgtgga
tggctttcccattgtgtcaacttgagcacagtggtgccagcccccatcccacttttgcaa
cctccattccttactccatggccattcttacctgttaccacctcttcctggcccttctct
atctggtctgtagcaccccaaacataccctttgccattttgaacctaatctactccagtc
caatccctagttccaaaccctagcccaggccctgggaaattcagatgtgggattagagag
gaagttcaaggttcatctgtcttttctctccagtcctaaaccttctttggttacaggtgg
catatgaacttctgggtgtgttgcgctcatctggaggagcggtgctgagcccccgggagc
tgcgggtctgggcccctctcttctctcagctgggcctccgcttccttcaggagctgtcag
agccccagcttagagccatgcttcctgtcctgcagggaactagtgttacacctgctcagg
tttgcctgtctcactccctggcatgtaccctccatccccgcttgagccccagtcaagaga
atcccattcagggataaaagcagcccctcctttccctgggtgaacagtagaggtaaactc
tgtctgcaggaggacgccttcattccctttcctcagatcaagaagggacctgagtcactg
aggatggttactagggatggttaagaggcagcgggaagttttggagggtttgccttagga
acccacttaggacctggctgctgggtcctgagagctgttgttttcggtcccatcccaaca
caggctgtcctgctgcttggacggctccttcctaggcacgatgtgagtagcagcaacttc
tcagcctcccgccagaggtctctatcctcttttaacctggctcctgcatctgcccctcct
ctctctccgctcccctcatacttactgccttgctgcattgtgattgttgtcttccccaac
acccttcccttcttcttcaggcctcttgtctctcttgctctttagctatccctggaggaa
ctctgctccttgcaccttctgctaccaggcctcagcccccagacactccaggccatccct
aggcgagtcctggtcggggcttgttcctgcctggcccctgaactgtcacgcctctcagcc
tgccagaccgcagcactgctgcagacctttcgggtatgagagtggcaaggaggatgagat
aatcagggataccggctctttctggttgggaggaaggcatcttccctgaggccagggaag
gcctttcatacctccccacttacacacacacacacacacacacacacacacacacacaca
accaattctcatgcaggttaaagatggtgttaaaaatatgggtacaacaggtgctggtcc
agctgtgtgtatccctggtcaggtaagtgtgagatctcccaactgagctcctctccccat
tctggggcagtttcatatggctggtgctacctcccacactaccctgcagtggccctgaga
gttctggttagctctgtgcccattagcagccctccccagtgccagatgcaggacagcatg
atccactcacattgtcctagactaatgtcaaagctggaagggcctgagaaatcttccagg
ccacccaccctgctttcagatgaaaagaccaaggctgggagaagctaagggactttgttt
gcctggtgcctaactagcagcaacacttgaccacagcagcctgcagtgtgaggctcttag
gcgtttattgctacagtggcaaatgccattccacttctgtcctagctttggtccctttcc
acccccatggttccttttctctgagtgctaagtacagactctctcacctatcactacact
gctatacccatcaccgccagcagcctattcccaccacctggccagactgcctgcttcccc
tgctcccattaaagctgctacaactggattccttggctcttctggcaaatcgaagacgct
actgggagctgccctggtctgagcagcaggtaattctccccacttaatttcagaacttcc
tccctcaatgtagtctaccttctttacctatcccttagccctatttggccagcttatccc
tactatcctttatttgattgtttgagatacagtctcactctgttgcccaggctgcagtgc
agtggcatgatcagagttcgctgtaacctcaaactcctgagctcaggcaatctttctgcc
tcagcctcctgaatagctaggacgacaggtggttaccaccatgcctggctaatttttaaa
ttttttttttgttttttgagatgaagtcttgctctgtcacccaggcttgagtacagtggc
acaagcttggctcactgcaacctctgtctcccgggttcaagcgattctcctgcctcagcc
tcccgagtagctgggactacaggcactccccacaatgcctggctaattttttttttgttt
tagtagagacagggtttcaccatattggccaggctggtctcgaactgctgaccttgtgat
ctgcctgcctctgcctctcaaagtgctgggattacaggtgtgagccaccatgcccggcca
atttttaaattttttgtagagacagacaatacaaaaatgtggacactatgtggagacact
atgttgaggtactatgctgtccagattggtcttgaactcctggcctcaagcaatcctcct
gccttggcctcccaaagtgctgggattacagacctgagccactgcacccagccccctagt
atctcttataatgtgacttgcttttctttttctttctccttcccttttctttcatttctt
tctcactctcgagagaagagtgggcatctgggagagtgggaggctggtgggtcccacaga
gtgaggaggcaggactgggtccaaggcagtcctgcctctccactctagggggtatccttg
gacagtgtctcttctgggaaggggctcgtctttctttctcttgtaggcacagtttctctg
gaagaagatgcaagtacccaccaaccttaccctcaggaatctgcagtgagtaacttgtgt
tgagcagtgcgctgaattcgaccaacatttttttgagtgcttactatgtgccaggcacca
tgtgatatggaatgggggatatagggatgaatgatgcatagtccctgcctcgtggacgtt
ctcctagcacctccctttgccctcctttccttccacagtgccatgcctatcctgactaga
gccaaaggactcagaaaacctggattcaggttccagtcctgtcacctacttgtcctcttg
ggcaagtcatttaacgtccctgtgtcagttttcccttctttaaatgagaattacaatggc
accagcctcataggtagttactgtgaagattaaatgaggtaggtcatgtaagatatttaa
cacagtgtttggtccattgtaaagtcccagtagtcatttgctactgttagtttacttcag
gatgacttcagaggcactggccaagcaagaataaataggaataagaaggtatcactttac
ttacacccacattagaagaacaatgggcttcagaatctttttttttttttttttttcgag
acagtcttgctctgttgcccaggctggagtgcagtggcgcgatttcggctcactgcaacc
tctgcctcccaggttcaagcgattctcctgtctcagcctctggagtagctgggattacag
gaatgtgccaccatacccagctaatttttgtatttttagtagagatggggtttcaccatt
ttggccaggctggtctcaaactcctgacctcaggtgatccacccgcctcagcctcccaag
ggcttcagaatctaagacatggctctagtttcagtttaccacatttctagcagaatgatg
ttgggaatgtcacctgacttccataaatccttattttctcctctgataaacagcagtgat
gttatggggagctgatgagatatctatgtaaaaacatttctcaaaccataaattacggtg
gatgaacatctgtacttgtgttgagagtactgatatcaaggagcaaacaggctgttgtat
gtgttgaatgagcctctccccactcacacacccacagggctctgggcaccctggcaggag
gcatgtcctgtgagtttctgcagcagatcaactccatggtagacttccttgaagtggtgc
acatgatctatcagctgcccactagagttcgagggagcctggtgagagggggtgcctgga
ctttagtgggagcagggaggctgggaccctaggtatagaacccagctcctatgttctgct
ctggcctcacactgcttccctacagagggcctgtatctgggcagagctacagcggaggat
ggcaatgccagaaccagaatggacaactgtagggccagaactgaacgggctggatagcaa
gctactcctggacttaccgtaagtactgcagctagagatattggcccctcagaaagctca
atctggggtgaagatctgcccttagggaatgccctggaggaggtagtttttctgtctggt
agttccctgacataatttatagcccaaagcagaggattttattcaaagttgctctatgta
ttgactggttcccagaatatgctccagcacagggcagctgagggtggcaacactgtattg
aagcctgccaagtaatcttacaataacctagtccacattaattgagattgagacagagca
tctgaagtgagggaggcaatgctccaaatctgccccagaggattgtagtttgctcagggc
actgtgttcttagtgcattcagaggagtagatcgagagaaaaatatatgaaaaatgtgat
aaataccttcaaatacctgaggggctatcaagtagaaattagattgtcatatttatgagt
ggccccattgggcaagactaagagtagttaacggagatcagatttttacatagtataaga
aaaactaaggtagtgagttcctggtccttggagctgttcgagcctaagccagatggcccc
atggcaggaatgttgtagagcacgttcatatacaggttgtgggaagaaaaggctatagga
acccaaggctcctccctacccatggagaaatttattagtatgttactcatatgctgcttt
tctcattttacccctaccaccaccccgttgccatccgcactgtaagtcaggataggaaaa
tgctggtgttacagtcttcctggggaatatggagctgaagtggagtaaaagcagttgact
tcattcctacttttttcttttttttctttttttttttttttgagacagagttttgctgtg
tcaccaaggctggagtgcagtgacgtgatctcggctcactgcaacctccatcttccaggt
tcaagcaattctcctgcctcagtctcccgagtagctgggactgtaggtgtgcaccaccat
gccaggctaatttttgtatttgttgtagggacgagctttcaccatgttggccaggctggt
cttgaactcctggcttcaagtgatctgcccacctcggcttcccaacattcttatattttt
ataggcctttccacagatttcagctcttgtatgacttagcccagttccagaactggtaat
cctaggtagggtacaggttatcacctctgatttcgggtaaaagggatttatttatttatt
tgtttatttatttatatttttgagacagagtctcgctctgtcacccaggctggagtgcaa
tggtgccatctcggctcactgcaacctctccctctggggttcaagcaattctcctgcctc
agcctgctgagtagctgggattacaggcgcgtgccaccacacccggctaatttttgcatt
tttagtagagacggggtttcaccatgttgctcagggtggtctcgaatttctgaccctgtg
atctgcctgcctcggcctcccaaagtgctgggattacaggcatgagccactgcgtccggc
ctgtttttacttttttttaatgccattcagatctgtttaaatatgtgggttctgtgagat
aatttagaatcccaaggttacagatgaggtgaaagatcctagaccatgcatcaaaaaact
tgagtttctcatttgtgaaagaaggataagagaaacacctattttgtctgggtgcagtgg
ctcatgcctataatcccagcatttggggaggccaaggtgggtggatcacggaggtcaggt
gttcaagaccagactggccaacatggcaaaacaccatctctactaaaaatacaaaagtta
gctgggcgtggtggcacgtgcgtgtaattccagctattcgggaggctgaggcacgagaat
tgcttgaacctgggaggtgcgggttgcagtgaactgagatcgcagcaccactgtgctcca
gcctgagtgatggagtgaggccaggtcttgttgtaggatcaaatgagataacacctgaaa
gaactttgtaaattgtatagcacgtacaaacaagaagggacctcttcacaagcagaggaa
gggtggtcctgtggaaaaaaacgggaattgggagtgagagacctcaacatttgatctctg
tgaacctcagttttttaatctataaaatggggaaatgttaatggtacttaatatttggag
cttttgagtccattagatcaggtaggattgttcgttattttttttttttaggaagactag
aaatatgttgctccctttttctcccccactcaagcttgatggtgggaattggccctggag
ctgtttactatcagttcctgtccagcttcactaaatttggtctggggtcacatcttagct
gcggactgtggggttttgtggtcccttctcgacttggcccagctccacctgaatcctgtt
gttgtcaaattgctgtaataggatccagttgatggacagactatccaatgaatccattat
gttggtggtggagctggtgcaaagagctccagagcagctgctggcactgacccccctcca
ccaggcagccctggcagagagggcactacaaaacctggtaagagtccaccctaccagact
cagatttgctgccctgggcaattcttgctcctcagacaatgctctctgactgtcccccaa
ccctctacttcttgctttcttgctgccaaacagattcctgtctacaaggcctggcccctg
ttttgcctctgggttctgttccttgataatatgcttcacgttacttgtccatacctcttg
gagtccgagaaatctcttggagtccacctctcagtctttctgcctgctcctatctgggct
cattgcttaaggaagtgaacaaaggtagtgagcatcatagggtgctgagctgggagcagg
agggagggaaggttagggggcttggtgtcttgatcaaggtgtctggtattctgagtcaga
agtgcattgtccaagttctgatgctcttctccaggctccaaaggagactccagtctcagg
ggaagtgctggagaccttaggccctttggttggattcctggggacagagagcacacgaca
gatccccctacagatcctgctgtcccatctcagtcagctgcaaggcttctgcctaggaga
gacatttgccacagagctgggatggctgctattgcaggagtctgttcttgggtatggacc
ttcgagaacttcagattctaactcattctatacccagtccctcagccaccatcatcagtg
gcagcctgttccatattcttaaggtcccctggagccctgtgtccgaaatcctagcatgtc
ctcttttccccttccttttcctcacagttccctcagctccccagcccccgattttcttcc
tgtccccaggaaaccagagttgtggagccaggatgaagtagagcaagctggacgcctagt
attcactctgtctactgaggcaatttccttgatccccagggtgagatgaaggaagaaggg
aagggagtaaatgcatagaggggactggtgagctggttatggggacccgtggccaaagag
ggcaaaggatatgaagcctagatctggggggagactgcaaaacagagacaggactttgga
cttagagctatagcagcaggtcctgatctgtccagatctccccactctccttctaccttc
tcatgcaggaggccttgggtccagagaccctggagcggcttctagaaaagcagcagagct
gggagcagagcagagttggacagctgtgtagggagccacagcttgctgccaagaaagcag
ccctggtagcaggggtggtgcgaccagctgctgaggatcttccaggtgaaactacccaaa
tacttatatgtccagcaggatgtacagggagtatcaaacggtctgggttctacatgtgct
cttccctgggactgggttttctaatttataaagcaaagagtttagagggatgatcttcaa
gcctcttgtagttctagaattctgtagttctgggagtttgtaaactattaagttttcttt
tagcccagaacttccattttcctgctctctcgtgtctgctctagactcagctctagctcg
gctaagtgtggagctctctgctggggagatccctagaagctttgaaggagacattgtgag
gctggagaactgggttcaaattcagtgctaccattaaatctctgaataacatcctcagtc
ttccatctataaaagtcttggcatctccaatcacttcttgttctattatctcctaagccc
tatacatattactctgtaatactcctttgatccctatttctcacagtgctctatcctcca
aaggttggaagactcactctatctacagatatctctctgggcatattttattactgcgct
gacctcctggccctgccttcccccttcagaacctgtgccaaattgtgcagatgtacgagg
gacattcccagcagcctggtctgcaacccagattgcagagatggagctctcagactttga
ggactgcctgacattatttgcaggagacccaggacttgggcctgaggaactgcgggcagc
catgggcaaagcaaaacaggttagggatggagagccaactggggttggccatgaggaagc
tatttgggtgtgatgtaggacacaaagagaatggagagttggatgagaggtgggggaagc
aagagatagaagagttagaagatttgggtcacaagtaggaggtgaagggagataaatatt
gaggaaagagagctagtataatgaatagagggacgaaagcagtggttaccaaattttaat
gcatatcacgatcatcaagggaacagatttttttctttatttttttttctttcttaaaaa
aataatggcatgcttcggctgggtgcagcggctcacgcctataatctcagaactttggga
ggccaaggcgggcagatcacgaggtcaggagatcaagaccatcctgtctaacacggcgaa
acacggtctctactaaaaatacaaaaaagttagccgggcatggtggtgcacacttgttgt
cccagctacttgggaggctgaggcaggagaatggcgtgaacctgggagggggagcttgca
gtgagccgaagtcaagccaatgcactccatcctgggtgacagagcaagactccatctcaa
aaaaaaaaaaaaaaaaaaaaggcatgcttcatgaatttgcgtgttatccttgcacaggcg
ccatgcaaatctctgtatcattccaattttttggggtatgtgctgctgaactgagcatgg
gaacagtgccagtgccagattaccatgcttcactgacttaataaaaacctttggggaggc
tgggcgcagtgactcatgcctgtaatcacagcactttgggaggcggaggcaggtggattg
cttgagcccaggagttagagaccagactgggcaacatggtgaaaccctgtctctactaaa
aatagaaaaaacattagctgggtgtggcggcacatgcctgtaatcccagctactcaggag
gctggggtaggagaatcccatgagtgcaggaggtggagggtgcaatgtgccaagatcgca
ccactgccctccagcctgggtgtcagagcaagaccctgtctcataaattaaaaaataagc
ctctgggggaaagagtctagacatctgcatctcctttttttttttttttttttttttttg
agacagagtctcactctgtcacccagcatccaggctggagtgcagtggtgtgatcttggc
tcactgtaacctctacatcctgggttcaaacgatcctcctgcctcagcctctcaagtagc
tgggactacaggtgcaccacacctggctaatttttgtatctttggtagagatggggtttc
actatgttgcccaggatggtctcgaacttctgggctcaagcaatcctcccacctcagcct
cccaaagtgctgggattacagctgttagccactgtgctgggccctaggcatctgttttaa
taagcgtctctgtgtctgatgcacataaaagtgtggaactcatggactagagttagtttg
ctcttcttttccactgattgtaatgtctttcaaaacaccttagaggaactgtaaggcaac
ggtctcattttatagtggaggaaactaaagaaaaggcaaatgatttacctagagttatac
agctaagggcagaggcaagacttaaaacccagcagtatgactcccaatccactgcttttc
cactcacattgttcctgtctttctcctagttgtggggtcccccccggggatttcgtcctg
agcagatcctgcagcttggtaggctcttaataggtctaggagatcgggaactacaggagc
tgatcctagtggactggggagtgctgagcaccctggggcagatagatggctggagcacca
ctcaggtaacacttttcctcctccctacggcttcccaaacacccatcccacagacccagc
cctatagatcatctaaagcccaaggaatttttttcctgtgaccctacctggtccttcttt
ctatcttttgttgataccccatactagtgaccttcaggactctgatttattcactctgag
gccctggacacataatactgtctcctacctcttttcctggaggcttcctctttttctttc
cttttcttttctgagtcctcagccttccccatgactccttaggtcttaatagtaacagaa
tataacccagtaacacctatcacttccctgtccattaattctccataactttcctccttc
ccctcttctcccaccccccaccccagctccgcattgtggtctccagtttcctacggcaga
gtggtcggcatgtgagccacctggacttcgttcatctgacagcgctgggttatactctct
gtggactgcggccagaggagctccagcacatcagcagttgggagttcaggtcatttgtga
aggggctgagggtggtggtgctgaggtaaaggtggacttactggggaaagaaggatcatg
aaggtctggtcccatggaggaagggaactcatttgaagccatctcttcctttgtctcatg
accacagcccctttcactgaagccgaattcttcttccttccttcctactgttctacagcc
aagcagctctcttcctcggcaccctgcatctccagtgctctgaggaacaactggaggttc
tggcccacctacttgtactgcctggtgggtttggcccaatcagtaactgggggcctgaga
tcttcactgaaattggcaccatagcaggtggggagctgggccactgctggtgcaagttgg
tttggtttctataccatgggtggactggatggaagactgccctgcaattcttaaggtggg
ggcctgagggtgtttaaataaggggctagagacatattggggaaggtctatgatagggca
ctttgggagtagttagagaaggtctataggtttgaagagagggaaggtcagtctaagaca
atgtttggatgccacttgcttcaacagctgggatcccagacctggctctttcagcactgc
tgcggggacagatccagggcgttactcctcttgccatttctgtcatccctcctcctaaat
ttgctgtaagtattaatggactggggtgaccacaggagagccagggcccaatggggacta
catgcatgcactgattcctacccctgccctcaggtggtgtttagtcccatccaactatct
agtctcaccagtgctcaggctgtggctgtcactcctgagcaaatggcctttctgagtcct
gagcagcgacgagcagttgcatgggcccaacatgagggaaaggagagcccagaacagcaa
ggtgagttcccagctgcacagcttgatcctccatctcctgacccagaatcaaacccctaa
tttggtgctgtctggctcttagagtgcacccagggagatccctggagtgaaggagtctac
aggcagagcgctaatttccaagtatcaatgctcctggagagctgagttgtgatattactc
ccattccctgtctattataggtcgaagtacagcctggggcctccaggactggtcacgacc
ttcctggtccctggtattgactatcagcttccttggccacctgctatgagcctgtctcta
cagtagaaggagattgtggggagagaaatcttaagtcataatgaataaagtgcaaacaga
agtgcatcctgattattttcagaagctgatgaggaata

By “human Myo7A promoter” is meant a polynucleotide that encodes a human Myo7A promoter region. An exemplary Myo7A promoter nucleic acid molecule is 95%, 96%, 97%, 98%, 99%, or 100% identical to the following sequence (SEQ ID NO:4):

>Human_MYO7A_Promoter_Sequence
ccaggcaggtaaggatcaggggtgataggggagagcaattactttgttcactgtatgcac
ccgcgggggcctgggagtccccatttgcaggtgggtagggcctccagcccacaccaccca
gacctaggcttccctcttctcaggatccaccacagggttaggggacaggaagcctgttct
attctcaataaatcttacaaaattccaaaaagactttcctgtgtccctctgtccccctgg
ggggaagggacaatggaggaatattccccaggcctgggcgactgtccgctggtcagaggg
aaggccccgctgcctgcccggtgcacctgtgagctgagattgtggggatcattcagtcat
tcctttattcagtagatctgtacggggctggctttgtgccaaggcctgccccgggcacct
gggatgtggggaaccaggcatagcaatccctgctctgggacagtctacaaacgagcagcg
agaacaaagacatactctggtcatgacagacactaccccgcacattactgatggggcagt
gtgggcctgctgggttagagacggcctactccctgatctgtgcatgaagggctgggaaga
ggagatgggccttcggggcacaggaaggtgtggggggaccagagcggaggctgctgaggg
cagatgcaggcatctagggccaggaagctggtgtggtggtggggagaagcagggttggga
tgactgaaagcagagtggacgactcaatgaaaaacgacttgggagacggggtgagtgcag
tgtgagggcaaagtgaaccgagatgcctcgctttcttcatgggactgagaaatgggtatt
gcaaatcgtgtgatctgcttagcatagtgcctggttcatagcatggctgaacacaatgta
gctgtcatcctggtgtttggaggccgccctgtcccccataacctgcccacctctctgccc
cattcccacagctcactgcattttgtccctgctgccagtcgtgtgccatgggctctcctg
tgctcccccggggtctcatttccccatccgtgtagggctgacgccactggccccgcccag
cctgggaggatggagaggggggacttgaggagtggccagtactgggtttcggcagcaggt
gcaaatcccaggtggggactgtgttccagggactcctggactcctgttaggccagcaggc
taaggggcagacggcatcctggggtcttcctgggacagggtcgtcgggtctgaggggacc
caggccctagcagaagctgactcctgtcacggtcacttcccagcggtgctcagcccgcct
gacggaagcagctgggcagtggggcctgtgaccagcaggcggcgctggcgagctatagcg
cccctttgggcccccaccccaccttctgggcagcttccctgcagacaccccagttatggg
ggctagggacccaaaagagacatccttctgccacccagagctgccctggcgaggtgcact
atggggccgccgacagctgcgtggctgccgagggcggaaaggagaaactgtcatgtcccg
atagggccgcgcgaggtctccatcctcgacaacgctaataacaaagacgtgtgctcctct
ttgcttggttccccccactcctttaaatcacagatttcacttcagtttatctgtgtcgct
gtcacacgtggggtggctcccagtcagctggtttggcaaagtttctggatgattacggaa
taacatgtgtccccaacccgcagagcaggttgtgggggcaatgttgcattgaccagcgtc
agagaacacacatcagaggcaagggtgggtgtgcaggagggagaaggcgcagaaggcagg
gctttagctcagcactctccctcctgccatgctctgcctgaccgttccctctctgagtcc
caaacagccaggtagaggaggaagaaatggggctgagaccccagcacatcagtgattaag
tcaggatcaggtgcggtttcctgctcaggtgctgagacagcaggcggtgtcctgcaaaca
acaggaggcacctgaagctagcctggggggcccacgcccaggtgcggtgcattcagcagc
acagccagagacagaccccaatgaccccgcctccctgtcggcagccagtgctctgcacag
agccctgagcagcctctggacattagtcccagccccagcacggcccgtcccccacgctga
tgtcaccgcacccagaccttggaggccccctccggctccgcctcctgggagaaggctctg
gagtgaggaggggagggcagc

By “human Myo7A enhancer” is meant a polynucleotide that encodes a Myo7A enhancer region (e.g., the intron 1 enhancer). An exemplary human Myo7A enhancer nucleic acid molecule is 95%, 96%, 97%, 98%, 99%, or 100% identical to the following sequence (SEQ ID NO:5):

>Myo7A_enhancer
agtgctggctggacagctgctctgggcaggagagagagggagagacaagagacacacaca
gagagacggcgaggaagggaaagacccagagggacgcctagaacgagacttggagccaga
cagaggaagaggggacgtgtgtttgcagactggctgggcccgtgacccagcttcctgagt
cctccgtgcaggtggcagctgtaccaggctggcaggtcactgagagtgggcagctgggcc
ccaggtaaggatgggctgcccactgtcctgggcattgggaggggtttggatgtggaggag
tcatggacttgagctacctctagagcctctgccccacagccacttgctcctgggactggg
cttcctgccacccttgagggctcagccaccacagccactgaatgaaactgtcccgagcct
gggaagatggatgtgtgtcccctggaggagggaagagccaaggagcatgttgtccatcga
atcttctctgagctggggctggggttagtggcatcctggggccaggggaatagacatgct
gtggtggcagagagaagagtccgttctctctgtctcctttgctttctctctgacactctt
tatctccgtttttggataagtcacttccttcctctatgccccaaatatcccatctgtgaa
atgggagtatgaagccccaacagccagggttgtagtggggaagaggtaaaatcaggtata
gacatagaaatacaaatacagtctatgccccctgttgtcagttggaaaagaaattaactt
gaaggtggtctagttctcatttttagaaatgaaatgtctgtctggtcattttaaaatgtg
gcccttaaatttcacgccctcaccactctcccccatcccttggagccccatgtctctagt
gaaagcactggctctgcccccagccctcatggctcatgctggcatagggcgcctgctcca
cagcctgggcaccatcttcagacaagtgcccggtggcaactgcctgctggccctgttgaa
tccacatctccaccaggcatccagactagttcaggtctctggaaggactgtgggtttgct
gtgtcccagagctccagggcaggggtcagggctcggatgtcgggcagtgtcatgggcaga
ggatcgaatgccccggcggctctgaatgggcccttgtgaaaaattgatgcgcattctagg
agacaggttgggagccagaggggcctcataccagggtctgtaggctggggctgcctttta
agctccttcctgaggccgtctctgggtctggccctgtgctggacaaggctggagacaagg
Caatgtctcagaccctctcccattggccacatcctgccctggatcaactcgccaactttg
ggggcagaggtgggactgacccttaccctgacaacataatgcatatagtcaaaatgggat
aaaggggaatatagaggctcttggcagcttgggagtggtcagggaaggcttcctggagga
ggtatcatctgaactgagccatgaaccataagtggaaattcactagtcaaaatttcaggt
agaagggccagtgtgtgaaggccaggagatggcaagagctggcgtatttcaggaacagtg
agtcactgaggatgtccaagtataagggtaggaaagggagtgagcagtgagagaaaagac
cgaggcatcagcaggggccagattgtgctgggcctagcggggcgggcccgggcccgggcc
caggcccaggtgcggtgcattcagcagcacagccagagacagaccccaatgaccctgcct
ccccgtcagcagccagtgctctgcacagagccatcctgagggcagtgggtgctcttgaga
ggtttcaggcagggtgtgctgtgagcaggtcatgcccagcccttgaccttctgctcagtc
aggcttgtccttgtcacccacattcctggggcagtccctaagctgagtgccggagattaa
gtcctagtcctaaatttgctctggctagctgtgtgaccctgggcaagtcttggtccctct
ctgggcccctttgccgtaggtccctggtggggccagacttgctactttctaggagccctt
tgggaatctctgaatgacagtggctgagagaagaattcagctgctctgggcagtggtgct
ggtgacagtggctgaggctcaggtcacacaggctgggcagtggtcagagggagagaagcc
aaggagggttcccttgagggaggaggagctggggctttgggaggagcccaggtgacccca
gccaggctcaaggcttccagggctggcctgcccagaagcatgacatggtctctctccctg
cag

By “mouse Myo7A promoter” is meant a polynucleotide that encodes a mouse Myo7A promoter region. An exemplary Myo7A promoter nucleic acid molecule is 95%, 96%, 97%, 98%, 99%, or 100% identical to the following sequence (SEQ ID NO:6):

>mouse_Myo7A_promoter
agagggatctgtctgtttcatttttcccgtaccccgcacccccccccacacacacacaca
caagggtaaagacagagggcacaatgggtagctgacctctggtcagaaggatgagcaagg
aaggccttgggaatcacagataaaagctggccttgctggttacctagtgagtcagagctc
agctctgtggaatccttcaggttccctgtcagtgtggggctggctttgccgagctctgcc
catctcaggccctggggacatgggggcacacagttcctgctctgcagcagccttccagca
actgaggaaaatgtacaaatcatgtctgatcgaagtactgcagtgtccttgataggcagt
gtccctaggagctgactctcagtctctgagttcaagatctgtgccctgttctagtggaga
ggagagacttacatgcaggccacagacagaggtggcagagcagagatactgggaaggtgc
ggtagctagagctaggaatttatggccagggccacgggcagaagctgggttgggttgtgg
aaaacgaaatgatggaatacgacgatagattggatttggaggggcgagggtggtatgaac
ttaagaccagttctgcagtggttcagatgtggtgcccttaagaggctcaggtgccagaaa
ccagtgccccttctggatagggcagaaaacaggcagaacctggagaggtacggtggagca
tgaggtggtaaggacacacaccatctcaggtggtttgttttgggggaaacaagcatggat
ttctaggtttctccgatcgggtgacctgcttagccgagtttgggtgctgaggaaatgtct
ccgttatggttctgagaccactttctcacccacccactgccttcccagcatgcacctcag
ccccaacgaagcacacctgccccagttgccgccttgtcgattgggctctggatgctccaa
cctgtgtctctcctactctgtgtaggctcatgctactcacctgactcataagtatcagtt
tcgtaggtgaaggctgcgttgggtgcaagtcaaccttggaccgtggtctcactgtggcac
cctggtatgaggaagttgaccggcttttccttagtcttgtagcagttggctgtgcccagc
aggtggcaccattgcaccattgattctccccactccatacccactatagattccacccca
ccctattcccctccccaccttaacccacccacccccaccgaaaagcagctttcctgagta
gatgtcccagctggcaagtgtgggcagaagaaggggccaggtctcaggaggaggaggagg
aggaagaggaagaggagtcctccatcctccatcctcatctaccatgatgagtatttgtgt
cctgttcattcccacccactcccttttttaaatcacacatttaaatcacacacacacaca
cacacacacacacacacacacacacacacacacacacacacacacacacacaccgggtaa
gtctgtcctgcgtgaggtggctcctactcaggtggctttgcaaactgtctggataacagc
acactcaagactcctaggacaggctgtgggggccagttgtagagcttgggggtggggtgc
atcttggggagtcctggtttggatgttgtgtccagccaaggctccaggtattgcaaagcc
tgctcatctatgtggtctctagtagtgccctgacgagggaagctgggtgagcaggggagg
ctactgggaactgagacccagcaaaatcatgaggaagatgggacgtgatcaggtgtccta
accatgcagagatggcaggtagtaacacatgtgacaagagaccctgaggtcctgatggtt
ggccccaggcccgaggttcccactggccagcagtgccccctggagcttctatgccttgca
tccctgctggttagctttacacagcaccttgggcaacctctagacgttagtcagcagccc
cagcacagcccgcccctcatgctgatgtcaccacatccagaccttcgaggcccccagggc
tccgcctcctgggagaaggcttt

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Unless otherwise clear from context, all numerical values provided herein are modified by the term “about.”

By “control” or “reference” is meant a standard of comparison. Methods to select and test control samples are within the ability of those in the art. Determination of statistical significance is within the ability of those skilled in the art, e.g., the number of standard deviations from the mean that constitute a positive result.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

Unless specifically stated or obvious from context, as used herein, the term “or” is understood to be inclusive. Unless specifically stated or obvious from context, as used herein, the terms “a”, “an”, and “the” are understood to be singular or plural.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, the terms “treat,” “treating,” and “treatment” encompass a variety of activities aimed at desirable changes in clinical outcomes. For example, the term “treat”, as used herein, encompasses any activity aimed at achieving, or that does achieve, a detectable improvement in one or more clinical indicators or symptoms of hearing loss, as described herein.

The transitional term “comprising,” which is synonymous with “including,” “containing,” or “characterized by,” is inclusive or open-ended and does not exclude additional, non-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed embodiments presented in the disclosure.

The embodiments set forth below and recited in the claims can be understood in view of the above definitions.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

In an aspect, the present disclosure provides an expression vector that includes a nucleic acid sequence selected from the group consisting of SEQ ID NO: 1, a nucleic acid sequence having at least 90% sequence identity to the nucleic acid of SEQ ID NO: 1, and a promoter operatively linked to the nucleic acid sequence.

In some embodiments, the expression vector is a Lentiviral vector.

In some embodiments, the expression vector is an adeno-associated viral vector such as, for example, AAV2, AAV2/Anc80, AAV5, AAV6, AAV6.2, AAV7, AAV8, AAV9, AAVrh8, AAVrh10, AAVrh39, AAVrh43, AAV1, AAV2, AAV3, AAV4, AAV5, AAV6, AAV7, AAV8, Anc80, or AAV50.

In some embodiments, the promoter may be an STRC promoter, a Myo7a promoter, a human cytomegalovirus (HCMV) promoter, a cytomegalovirus/chicken beta-actin (CBA) promoter, a Barhl1 promoter/enhancer, or a Pou4f3 promoter.

In one aspect, the present disclosure provides a pharmaceutical composition for use in a method for the treatment or prevention of hearing loss comprising an expression vector comprising the nucleic acid sequence of SEQ ID NO:1 or a nucleic acid sequence having at least 90% sequence identity to the nucleic acid of SEQ ID NO:1, wherein the nucleic acid sequence is operatively linked to the nucleic acid.

In one aspect, the present disclosure provides a cell comprising an expression vector comprising the nucleic acid sequence of SEQ ID NO:1 a nucleic acid sequence having at least 90% sequence identity to the nucleic acid of SEQ ID NO:1; and a promoter operatively linked to the nucleic acid.

In one aspect, the present disclosure provides a method for treating or preventing hearing loss, comprising administering to a subject in need thereof an effective amount of an expression vector comprising a nucleic acid sequence selected from the group consisting of SEQ ID NO:1, a nucleic acid sequence having at least 90% sequence identity to the nucleic acid of SEQ ID NO: 1; and a promoter operatively linked to the nucleic acid.

In some embodiments, the expression vector may be a Lentiviral vector or an adeno-associated viral vector such as, for example, AAV2, AAV2/Anc80, AAV5, AAV6, AAV6.2, AAV7, AAV8, AAV9, AAVrh8, AAVrh10, AAVrh39, AAVrh43, AAV1, AAV2, AAV3, AAV4, AAV5, AAV6, AAV7, AAV8, Anc80, or AAV50.

In some embodiments, the promoter may be an STRC promoter, a Myo 6 promoter, a Myo7a promoter, a prestin promoter/enhancer, a Myo15 promoter/enhancer, a human cytomegalovirus (HCMV) promoter, a cytomegalovirus/chicken beta-actin (CBA) promoter, a Barhl1 promoter/enhancer, or a Pou4f3 promoter.

In some embodiments, the cell is a stem cell. In some embodiments, the stem cell is an induced pluripotent stem cell.

In some embodiments, the expression vector is administered by injection into the inner ear of the subject. In some embodiments, the injection method is selected from the group consisting of cochleostomy, round window membrane, endolymphatic sac, scala media, canalostomy, scala media via the endolymphatic sac, or any combination thereof.

In some embodiments, the subject has one or more genetic risk factors associated with hearing loss.

In some embodiments, the genetic risk factors may be a mutation in the STRC gene.

In some embodiments, the subject does not exhibit any clinical indicators of hearing loss.

In one aspect, the present disclosure provides a transgenic mouse comprising a mutation/variation that causes hearing loss selected from a group consisting of a mutation/variation in the human STRC gene.

The subject matter that is regarded as the invention is particularly pointed out and distinctly claimed in the claims at the conclusion of the specification. The foregoing and other objects, features, and advantages of the invention will be apparent from the following detailed description taken in conjunction with the accompanying drawings.

### BRIEF DESCRIPTION OF THE DRAWINGS

FIG. 1 shows the location of the Stereocilin (STRC) gene on chromosome 15 from 15q13-q21.

FIG. 2 shows the mRNA transcription map of STRC.

FIG. 3 shows the mRNA transcription map of a STRC pseudogene.

LV-SIN FIG. 4 shows a linear vector map of an exemplary LV-SIN lentiviral vector, where GOI represents the STRC gene.

FIG. 5 shows a linear vector map of an exemplary LV-ctrl lentiviral vector.

FIGS. 6A-6D are a series of dotplots showing dTom expression in HEI-OC1 cells. In particular, the percentage of HEI-OC1 cells expressing the vector-encoded dTomato reporter and the STRC protein. Flow cytometry analysis was performed upon intracellular staining for dTom expression in non-transduced controls (NTC) and cells transduced with LV-ctrl or LV-SIN at MOI 2. The populations shown were pre-gated for live cells using SSC-A/FSC-A characteristics, followed by gating for single cells according to FSC-A/FSC-H characteristics. FIG. 6A shows data for NTC. FIG. 6B shows dTom expression at MOI 1.277. FIG. 6C shows dTom expression at MOI 3.278. FIG. 6D shows dTom expression at MOI 10.279.

FIG. 7 shows a fluorescent image of delivery of an exemplary human STRC gene to the inner ear of the mouse via an exemplary embodiment of a gene therapy construct in which a human cytomegalovirus promoter (hcmv-p)/STRC/dTom cassette is incorporated into a third-generation lentivirus pseudotyped with vesicular stomatitis virus (VSV-g) protein. Briefly, STRC transcription is controlled by the hcmv-p and the dTom tag facilitates detection of the expressed STRC protein. Robust delivery to the inner hair cells (arrow) and outer hair cells (stars) was detected.

FIG. 8 shows the distribution of pseudotyped LV-hcmv-dTom in the adult mouse inner ear. Delivery of 1×10\hat{ }6 PU to the posterior semicircular canal of a P30 C57Bl/6 mouse. Expression of dtom can be seen in all hair cells as well as in the spiral ganglion demonstrating the capacity of this vector to target the cells targeted by mutations in STRC.

### DETAILED DESCRIPTION

The present disclosure is based, at least in part, on the discovery that full length or near full length Stereocilin (STRC) gene may be incorporated into a lentivirus vector under the control of an inner ear specific promoter (e.g., a mouse or human Myo7A promoter) to generate robust expression of STRC in inner ear cells. The techniques herein provide the ability to rescue STRC loss-of-function mutations in mammals (e.g., humans) via gene therapy. The disclosure provides compositions and methods for restoring STRC function to patients suffering from disorders that result from STRC mutations.

### Overview

Hearing loss is the most common sensory deficit in humans. According to 2018 estimates on the magnitude of disabling hearing loss released by the World Health Organization (WHO), there are 466 million persons worldwide living with disabling hearing loss (432 million adults and 34 million children). The number of people with disabling hearing loss will grow to 630 million by 2030 and to over 900 million by 2050. Over 90% of persons with disabling hearing loss (420 million) reside in the low-income regions of the world (WHO global estimates on prevalence of hearing loss, Prevention of Deafness WHO 2018).

More than 50% of prelingual deafness is genetic (Centers for Disease Control and Prevention-Genetics of Hearing Loss). Hereditary hearing loss and deafness may be conductive, sensorineural, or a combination of both; syndromic (associated with malformations of the external ear or other organs or with medical problems involving other organ systems) or nonsyndromic (no associated visible abnormalities of the external ear or any related medical problems); and prelingual (before language develops) or postlingual (after language develops) (Deafness and Hereditary Hearing Loss Overview; GeneReviews; Richard J H Smith, MD, A Eliot Shearer, Michael S Hildebrand, PhD, and Guy Van Camp, PhD).

Hearing impairment is a heterogeneous disorder affecting approximately 1 of 1000 newborns. At present, 42 genes and 69 loci (http://hereditaryhearingloss.org) are implicated in nonsyndromic autosomal recessive deafness (locus notation DFNB). In the European population, 20-40% of nonsyndromic hearing loss (NSHL) is due to mutations in GJB2 (MIM: 121011) and GJB6 (MIM: 604418), together comprising the DFNB1 locus. With few exceptions, autosomal-recessive NSHL has similar manifestations, wherein hearing loss is severe to profound with prelingual onset initial candidate gene approach assigned STRC (MIM: 606440) to chromosome 15q15.3 encompassing the DFNB16 locus. Stereocilia form crosslinks necessary for longitudinal rigidity and outer hair cell structure, and upon mechanical deflection, stereociliary transduction sensitive channels open for cellular depolarization. Reverse transcriptase polymerase chain reaction (RT PCR) from several mouse tissues showed strong, nearly exclusive expression in the inner ear and upon knockout, these key structures were absent (Vona, B et al. “DFNB16 is a frequent cause of congenital hearing impairment: implementation of STRC mutation analysis in routine diagnostics.” Clinical genetics vol. 87,1 (2015): 49-55. doi:10.1111/cge.12332.).

STRC deletion frequencies of >1% have been calculated in mixed deafness populations and the incidence of STRC hearing loss is an estimated 1 in 16,000. Accumulating evidence suggests that DFNB16 constitutes a significant proportion of the otherwise genetically heterogeneous etiology comprising NSHL. One challenge impeding diagnostic implementation of STRC screening is the presence of a non-processed pseudogene with 98.9% genomic and 99.6% coding sequence identity residing less than 100 kb downstream from STRC in a region encompassing a segmental duplication with four genes, HISPPD2A (MIM: 610979), CATSPER2 (MIM: 607249), STRC, and CKMT1A (MIM: 613415). Apart from CKMT1A, these pseudogenes have mutations rendering them inactive. Homozygous deletions of STRC and CATSPER2 result in deafness infertility syndrome (DIS; MIM: 611102), characterized by deafness in both males and females, and exclusive male infertility, as CATSPER2 is required for sperm motility. Not only is it challenging to generate accurate sequencing data without pseudogene inclusion, it is even more difficult to interpret such data without the usual reliable resources for mutation interpretation, as these databases are ‘polluted’ with pseudogene data as well (Vona, B et al. (2015).

More than 70% of hereditary hearing loss is nonsyndromic. The different gene loci for nonsyndromic deafness are designated DFN (for DeaFNess). Loci are named based on mode of inheritance: DFNA (Autosomal dominant), DFNB (Autosomal recessive) and DFNX (X-linked). The number following the above designations reflects the order of gene mapping and/or discovery (Deafness and Hereditary Hearing Loss Overview; GeneReviews; Richard J H Smith, MD, A Eliot Shearer, Michael S Hildebrand, PhD, and Guy Van Camp, PhD). In the general population, the prevalence of hearing loss increases with age. This change reflects the impact of genetics and environment and the interactions between environmental triggers and an individual's genetic predisposition.

Sensorineural hearing loss (SNHL) is the most common neurodegenerative disease in humans and there are currently no approved pharmacologic interventions. SNHL can be caused by genetic disorders as well as acquired through injuries such as sound trauma and ototoxicity. Genetic diagnostics have demonstrated that there are at least 100 genes causing nonsyndromic sensorineural hearing loss, with the majority of causative alterations in the genes being single nucleotide variants (SNVs) or small insertions/deletions (indels). Recently, copy number variants (CNVs) have also been found to play an important role in many human diseases including neural developmental disorders. CNVs; i.e., alterations through the deletion, insertion, or duplication of approximately 1 kb or more of a gene, are thought to affect gene expression, variation in phenotype, and adaptation via gene disruption, which may impact disease traits. More recently, CNVs have been recognized as a major cause of SNHL. Shearer et al. reported that CNVs were identified in 16 of 89 hearing loss-associated genes, with the STRC gene being the most common cause of SNHL4 (Yokota, Yoh et al. “Frequency and clinical features of hearing loss caused by STRC deletions.” Scientific reports vol. 9,1 4408. 13 Mar. 2019, doi:10.1038/s41598-019-40586-7).

Clinical characteristics of hearing loss patients with detected CNVs were identified by a study of 1,025 subjects (age range, 0-70 years, mean age, 11.8 years). When classified based on age of onset as congenital-6 years, 7-18 years, adulthood (>18 years old), or unknown, most of the subjects with a causative STRC deletion were diagnosed with SNHL by adolescence. Causative homozygous STRC deletions were found in 14 of the 723 cases categorized as segregating autosomal recessive or sporadic (1.94%), and in 3 of the 264 cases with autosomal dominant inheritance (1.14%). Duplications (3 copies) of STRC were identified in 19 subjects (1.85%). It was unclear whether the 3 STRC copies were pathogenic or had any impact on phenotypes. Additionally, 27 subjects were identified with ST9RC heterozygous deletions defined as carrier deletions. The frequency of carrier STRC deletions was 2.63% ( 27/1,025) in the hearing loss cohort, which was identical (2.63%, 4/152) to that in the normal hearing controls (Yokota, Yoh et al. (2019).

The prevalence of CNVs in STRC among subjects in the study that were diagnosed with genetic hearing loss accounted for 5% 17/395) of all subjects. Moreover, when classified based on hearing level as mild-to-moderate or severe-to-profound, the prevalence of causative STRC deletions was 12% ( 17/140) in the subjects with mild-to-moderate SNHL. Consequently, CNVs in STRC were the second most common cause of mild-to-moderate SNHL after SNVs in GJB2. None of the subjects with severe-to-profound or asymmetric SNHL had disease-causing CNVs in STRC (Yokota, Yoh et al. (2019).

Recent advances in genetics and gene therapy techniques have shown that rescue of a number of recessive types of deafness is possible through gene therapy (Akil et al., 2012; Askew et al., 2015). Long term gene delivery to the inner ear has been achieved using adeno associated viral vectors (AAV) (Shu, Tao, Wang, et al., 2016). The first human clinical trial to reverse deafness using a gene therapy (CGF166) was initiated in June of 2014 and completed in December of 2019 (https://clinicaltrials.gov/ct2/show/NCT02132130). This trial evaluated the effects of overexpression of atoh1 in cochlear supporting cells to induce regeneration of hair cells. An alternate disease target for translational research in this domain is a recessive genetic hearing loss that affects a defined group of cells within the inner ear. Prevalence of the mutation within the general population and maintenance of normal cellular architecture are additional considerations.

There are currently no approved therapeutic agents for preventing or treating hearing loss or deafness. The current treatment options for those with disabling hearing loss are hearing aids or cochlear implants. Cochlear implantation is a common procedure with a large associated healthcare cost, over $1,000,000 lifetime cost per patient (Mohr P E, et al. (2000). The societal costs of severe to profound hearing loss in the United States; Int J Technol Assess Health Care; 16 (4):1120-35). The lifetime cost of a cochlear implants and hearing aids is prohibitive for most people and particularly for those living in low income regions (where the majority of persons with disabling hearing loss reside). Therapeutic options are needed to provide cost effective alternatives to cochlear implants and hearing aids.

As described herein, by carefully evaluating both the incidence of common recessive causes of hearing loss and taking into account the size of the gene and recent advancements in viral vector technology (i.e. carrying capacity), it is possible to develop a gene therapy program that has an accessible and fairly common patient population. For example, STRC is a major cause of congenital hearing impairment worldwide and is severe enough to require lifetime use of hearing aids and in severe cases, cochlear implantation.

### STRC

The STRC gene is a known deafness-associated gene causing mild-to-moderate hearing loss, and is a part of a large deletion in chromosome 15q15.3 at the DFNB16 locus. The STRC gene is part of a tandem duplication on chromosome 15; the second copy is a pseudogene. The two copies are in a telomere-to-centromere orientation less than 100 kb apart. The pseudogene is interrupted by a stop codon in exon 20 (e.g., n.t. 4057C>T; a.a. Gln1353Stop).

STRC contains 29 exons encompassing approximately 19 kb. STRC is made up of 1,809 amino acids and contains a putative signal peptide and several hydrophobic segments, suggesting plasma membrane localization. The predicted molecular weight of STRC post signal peptide cleavage is 194kD.

The Exon map of STRC including chromosome 15 base pair positions (negative strand) are shown in Table 2.

TABLE 2

| ID | Chromosome | Strand | Exon Start | Exon End |
|---|---|---|---|---|
| 161497 | 15 | — | 43599243 | 43599671 |
| 161497 | 15 | — | 43599563 | 43599671 |
| 161497 | 15 | — | 43599672 | 43599760 |
| 161497 | 15 | — | 43599960 | 43600107 |
| 161497 | 15 | — | 43600196 | 43600293 |
| 161497 | 15 | — | 43600534 | 43600682 |
| 161497 | 15 | — | 43600872 | 43601014 |
| 161497 | 15 | — | 43601396 | 43601551 |
| 161497 | 15 | — | 43603242 | 43603411 |
| 161497 | 15 | — | 43603996 | 43604152 |
| 161497 | 15 | — | 43604361 | 43604451 |
| 161497 | 15 | — | 43604650 | 43604846 |
| 161497 | 15 | — | 43605264 | 43605399 |
| 161497 | 15 | — | 43607863 | 43607975 |
| 161497 | 15 | — | 43608080 | 43608203 |
| 161497 | 15 | — | 43609276 | 43609334 |
| 161497 | 15 | — | 43610312 | 43610437 |
| 161497 | 15 | — | 43610312 | 43610440 |
| 161497 | 15 | — | 43610919 | 43610984 |
| 161497 | 15 | — | 43611148 | 43611315 |
| 161497 | 15 | — | 43611499 | 43611537 |
| 161497 | 15 | — | 43611842 | 43612024 |
| 161497 | 15 | — | 43612377 | 43612509 |
| 161497 | 15 | — | 43612791 | 43612906 |
| 161497 | 15 | — | 43613045 | 43613306 |
| 161497 | 15 | — | 43613887 | 43614053 |
| 161497 | 15 | — | 43614196 | 43614312 |
| 161497 | 15 | — | 43614414 | 43614476 |
| 161497 | 15 | — | 43615433 | 43616690 |
| 161497 | 15 | — | 43617458 | 43617482 |
| 161497 | 15 | — | 43617458 | 43617509 |
| 161497 | 15 | — | 43617571 | 43618356 |
| 161497 | 15 | — | 43618659 | 43618722 |
| 161497 | 15 | — | 43618659 | 43618770 |
| 161497 | 15 | — | 43618723 | 43618770 |
| 161497 | 15 | — | 43618723 | 43618800 |
| 161497 | 15 | — | 43618885 | 43619665 |
| 161497 | 15 | — | 43622241 | 43622716 |
| 161497 | 15 | — | 43709784 | 43709909 |
| 161497 | 15 | — | 43710391 | 43710456 |
| 161497 | 15 | — | 43710610 | 43710777 |
| 161497 | 15 | — | 43710961 | 43710999 |
| 161497 | 15 | — | 43711304 | 43711345 |
| 161497 | 15 | — | 43711346 | 43711486 |

mRNA transcripts found to correspond to the STRC gene are shown below in Table 3. In some embodiments, the STRC gene comprises the Q7RTU9 sequence.

TABLE 3

| | | Length | | | | |
|---|---|---|---|---|---|---|
| Transcript ID | Length (bp) | protein (aa) | Translation ID | Biotype | Uniprot ID | RefSeq Match |
| ENST00000450892.7 | 5515 | 1775 | ENSP00000401513.2 | Protein coding | Q7RTU9 | NM 153700.2 |
| ENST00000541030.5 | 5305 | 1002 | ENSP00000440413.1 | Protein coding | F5GXA4 | — |
| ENST00000432436.1 | 2259 | 663 | ENSP00000407303.1 | Protein coding | H7C2Q6 | — |
| ENST00000428650.5 | 5386 | 969 | ENSP00000415991.1 | Nonsense | E9PBT5 | — |
| | | | | mediated decay | | |
| ENST00000440125.5 | 4291 | 351 | ENSP00000394866.1 | Nonsense | E7EPM8 | — |
| | | | | mediated decay | | |
| ENST00000455136.5 | 1104 | 119 | ENSP00000394755.1 | Nonsense | H7C0F7 | — |
| | | | | mediated decay | | |
| ENST00000485556.5 | 4253 | No protein | | Retained intron | | — |
| ENST00000471703.5 | 3364 | No protein | | Retained intron | | — |
| ENST00000448437.6 | 2518 | No protein | | Retained intron | | — |
| ENST00000483250.5 | 571 | No protein | | Retained intron | | — |
| ENST00000470279.1 | 569 | No protein | | Retained intron | | — |
| ENST00000460952.1 | 543 | No protein | | Retained intron | | — |
| ENST00000493750.1 | 513 | No protein | | Retained intron | | — |

Stereocilin is expressed in the inner ear, nervous system, and CD14+ cells. The incidence of STRC deletions has been estimated to be between about 100 and about 500 in deaf populations (Yokota 2019). Mutations in the STRC gene are associated with Autosomal Recessive Nonsyndromic Hearing Impairment type DFNB16. The DFNB16 hearing loss is a major contributor to congenital hearing impairment. The clinical features of DFNB16 hearing loss are (OMIM 603720):
Autosomal Recessive
Mostly Congenital Presentation
Prelingual onset
Hearing loss is moderate to profound
Affects the high frequencies (e.g., high frequency sloping) and
Most likely to be stable over time

The STRC gene encodes stereocilin, a large extracellular structural protein found in the stereocilia of outer hair cells in the inner ear. It is associated with horizontal top connectors and the tectorial membrane attachment crowns that are important for proper cohesion and positioning of the stereociliary tips (OMIM 606440). The outer hair cell (OHC) bundle is composed of stiff microvilli called stereocilia and is involved with mechanoreception of sound waves.

In STRC null mice, the OHC bundle tip-links progressively deteriorate and fully disconnect from each other. Also, the tips of the tallest stereocilia fail to embed into the tectorial membrane. STRC is essential to the formation of horizontal top connectors, which maintain the cohesiveness of the mature OHC hair bundle. (Verpy 2011)

STRC deletion frequencies of >1% have been calculated in mixed deafness populations and the incidence of STRC hearing loss is an estimated 1 in 16,000. Accumulating evidence suggests that DFNB16 constitutes a significant proportion of the otherwise genetically heterogeneous etiology comprising nonsyndromic sensorineural hearing loss (NSHL) (Vona, 2015).

STRC Variants/Mutations on chromosome 15 known to cause hearing loss are described in Table 4.

TABLE 4

| VARIANT | | MUTATION | |
|---|---|---|---|
| NAME | MUTATION | TYPE | REFERENCE |
| NM_153700.2 | c.4701 + | Single | (www)ncbi.nlm.nih.gov/ |
| (STRC) | 1G > A | Nucleotide | clinvar/variation/165305 |
| NM_153700.2 | c.4195G > T | Single | (www)ncbi.nlm.nih.gov/ |
| (STRC) | (p.Glu1399Ter) | Nucleotide | clinvar/variation/165310 |
| NM_153700.2 | c.3670C > T | Single | (www)ncbi.nlm.nih.gov/ |
| (STRC) | (p.Arg1224Ter) | Nucleotide | clinvar/variation/165315 |
| NM_153700.2 | c.3670C > T | Single | (www)ncbi.nlm.nih.gov/ |
| (STRC) | (p.Arg1224Ter) | Nucleotide | clinvar/variation/179758 |
| NM_153700.2 | c.1086C > A | Single | (www)ncbi.nlm.nih.gov/ |
| (STRC) | (p.Tyr362Ter) | Nucleotide | clinvar/variation/228401 |
| NM_153700.2 | c.3217C > T | Single | (www)ncbi.nlm.nih.gov/ |
| (STRC) | (p.Arg1073Ter) | Nucleotide | clinvar/variation/228402 |
| NM_153700.2 | c.3493C > T | Single | (www)ncbi.nlm.nih.gov/ |
| (STRC) | (p.Gln1165Ter) | Nucleotide | clinvar/variation/228403 |
| NM_153700.2 | c.4057C > T | Single | (www)ncbi.nlm.nih.gov/ |
| (STRC) | | Nucleotide | clinvar/variation/242391 |
| NM_153700.2 | c.379C > T | Single | (www)ncbi.nlm.nih.gov/ |
| (STRC) | (p.Arg127Ter) | Nucleotide | clinvar/variation/505325 |
| NM_153700.2 | c.259C > T | Single | (www)ncbi.nlm.nih.gov/ |
| (STRC) | | Nucleotide | clinvar/variation/666998 |
| | c.4171C > G | Single | Francey et al. 2012 |
| | (p.R1391G) | Nucleotide | |
| | c.3436G > A | Single | Francey et al. 2012 |
| | (p.D1146N) | Nucleotide | |
| | c.4433C > T | Single | Francey et al. 2012 |
| | (p.T14781) | Nucleotide | |

Table 5 lists 31 patients that have the STRC mutation showing the name of the variant, genes affected, the protein change if any, the conditions that result and their clinical significance. The location of the mutation, the accession number and the ID of the patient are also provided.

TABLE 5

| | Genes | Protein | | Clinical | | | |
|---|---|---|---|---|---|---|---|
| Name | Affected | Change | Conditions | Significance | Location | Accession | ID |
| NC_000015.9: g.(4388 | CKMT1B, | None | Deafness, | Pathogenic | (GRCh38): | CV000562140 | 562140 |
| 6857_43888004)_(439 | CATSPER2, | | autosomal | (Last reviewed: | 43594659- | | |
| 84930_43992627)del | STRC, | | recessive | Aug. 6, 2018) | 43700429 | | |
| | CKMT1A | | 16 | | | | |
| GRCh38/hg38 | CKMT1B, | None | See cases | Pathogenic | (GRCh38): | CV000148737 | 148737 |
| 15q15.3(chr15: 43596729- | CATSPER2, | | | (Last reviewed: | 43596729- | | |
| 43659103) × 0 | STRC | | | Dec. 16, 2011) | 43659103 | | |
| NC_000015.10: g.(?_4 | STRC | None | Deafness, | Pathogenic | (GRCh38): | CV000004345 | 4345 |
| 3599438)_(43608225_ | | | autosomal | (Last reviewed: | 43599438- | | |
| 43613711)del | | | recessive | Nov. 1, 2001) | 43613711 | | |
| | | | 16 | | | | |
| NC_000015.9: g.(?_43 | STRC | None | Rare | Pathogenic | (GRCh38): | CV000165295 | 165295 |
| 891870)_(43910920_?)del | | | genetic | (Last reviewed: | 43599672- | | |
| | | | deafness | Jul. 14, 2015) | 43618722 | | |
| NC_000015.9: g.(?_43 | STRC | None | Rare | Pathogenic | (GRCh38): | CV000165297 | 165297 |
| 892732)_(43897597_?)del | | | genetic | (Last reviewed: | 43600534- | | |
| | | | deafness | Jan. 6, 2014) | 43605399 | | |
| NC_000015.9: g.(?_43 | STRC | None | Rare | Pathogenic | (GRCh38): | CV000165296 | 165296 |
| 892732)_(43893212_?)del | | | genetic | (Last reviewed: | 43600534- | | |
| | | | deafness | Mar. 4, 2014) | 43601014 | | |
| Single allele | CATSPER2, | None | Deafness, | Pathogenic | (GRCh38): | CV000560061 | 560061 |
| | STRC | | autosomal | (Last reviewed: | 43600609- | | |
| | | | recessive | Mar. 29, 2018) | 43647444 | | |
| | | | 16 | | | | |
| NM_153700.2(STRC): | STRC | None | Rare | Pathogenic | (GRCh38): | CV000228400 | 228400 |
| c.(?_4443)_(4845_?)- | | | genetic | (Last reviewed: | 43600750- | | |
| 68del | | | deafness | Feb. 11, 2019) | 43603344 | | |
| NM_153700.2(STRC): | STRC | None | Rare | Pathogenic | (GRCh38): | CV000180122 | 180122 |
| c.(?_4376)- | | | genetic | (Last reviewed: | 43600750- | | |
| 190_(4845_?)-68del | | | deafness | Nov. 28, 2014) | 43603601 | | |
| NM_153700.2(STRC): | STRC | E1613* | not | Pathogenic | (GRCh38): | CV000499237 | 499237 |
| c.4837G > T | | | provided | (Last reviewed: | 43600879 | | |
| (p.Glu1613Ter) | | | | Jan. 19, 2017) | | | |
| C | STRC | C1599fs | Rare | Pathogenic | (GRCh38): | CV000165302 | 165302 |
| | | | genetic | (Last reviewed: | 43600916- | | |
| | | | deafness | Mar. 15, 2014) | 43600920 | | |
| NM_153700.2(STRC): | STRC | None | Rare | Pathogenic | (GRCh38): | CV000165305 | 165305 |
| c.4701 + 1G > A | | | genetic | (Last reviewed: | 43601395 | | |
| | | | deafness, | Mar. 6, 2015) | | | |
| | | | deafness, | | | | |
| | | | autosomal | | | | |
| | | | recessive | | | | |
| | | | 16 | | | | |
| NM_153700.2(STRC): | STRC | R1468* | Rare | Pathogenic | (GRCh38): | CV000179758 | 179758 |
| c.4402C > T | | | genetic | (Last reviewed: | 43603385 | | |
| (p.Arg1468Ter) | | | deafness, | Apr. 27, 2018) | | | |
| | | | not | | | | |
| | | | provided | | | | |
| NM_153700.2(STRC): | STRC | E1399* | Rare | Pathogenic | (GRCh38): | CV000165310 | 165310 |
| c.4402C > T | | | genetic | (Last reviewed: | 43604384 | | |
| (p.Arg1468Ter) | | | deafness | Nov. 6, 2013) | | | |
| NM_153700.2(STRC): | STRC | Q1353* | not | Pathogenic | (GRCh38): | CV000242391 | 242391 |
| c.4057C > T | | | provided, | (Last reviewed: | 43604720 | | |
| | | | Deafness, | Aug. 29, 2017) | | | |
| | | | autosomal | | | | |
| | | | recessive | | | | |
| | | | 16 | | | | |
| NM_153700.2(STRC): | STRC | R1224* | Rare | Pathogenic | (GRCh38): | CV000165315 | 165315 |
| c.3670C > T | | | genetic | (Last reviewed: | 43608091 | | |
| (p.Arg1224Ter) | | | deafness | Nov. 28, 2014) | | | |
| NM_153700.2(STRC): | STRC | Q1165* | Rare | Pathogenic | (GRCh38): | CV000228403 | 228403 |
| c.3493C > T | | | genetic | (Last reviewed: | 43610317 | | |
| (p.Gln1165Ter) | | | deafness | Jun. 16, 2015) | | | |
| NM_153700.2(STRC): | STRC | W1162fs | Rare | Pathogenic | (GRCh38): | CV000179717 | 179717 |
| c.3484del | | | genetic | (Last reviewed: | 43610326 | | |
| (p.Trp1162fs) | | | deafness | Apr. 11, 2014) | | | |
| NM_153700.2(STRC): | STRC | R1073* | Rare | Pathogenic | (GRCh38): | CV000228402 | 228402 |
| c.3217C > T | | | genetic | (Last reviewed: | 43611237 | | |
| (p.Arg1073Ter) | | | deafness | Nov. 3, 2016) | | | |
| NM_153700.2(STRC): | STRC | None | Deafness, | Pathogenic | (GRCh38): | CV000004343 | 4343 |
| c.3156dup | | | autosomal | (Last reviewed: | 43611298 | | |
| (p.Cys1053fs) | | | recessive | Nov. 1, 2001) | | | |
| | | | 16 | | | | |
| NM_153700.2(STRC): | STRC | V724fs | Deafness, | Pathogenic | (GRCh38): | CV000004344 | 4344 |
| c.2171_2174del | | | autosomal | (Last reviewed: | 43614436- | | |
| (p.Val724fs) | | | recessive | Nov. 1, 2001) | 43614439 | | |
| | | | 16 | | | | |
| NM_153700.2(STRC): | STRC | Y362* | Rare | Pathogenic | (GRCh38): | CV000228401 | 228401 |
| c.1086C > A | | | genetic | (Last reviewed: | 43618042 | | |
| (p.Tyr362Ter) | | | deafness | Nov. 19, 2015) | | | |
| NM_153700.2(STRC): | STRC | R127* | Rare | Pathogenic | (GRCh38): | CV000505325 | 505325 |
| c.379C > T | | | genetic | (Last reviewed: | 43618042 | | |
| (p.Arg127Ter) | | | deafness | Sep. 1, 2016) | | | |
| GRCh37/hg19 | CKMT1B, | None | Deafness, | Pathogenic | Not | CV000625830 | 625830 |
| 15q15.3(chr15: 43890409- | CATSPER2, | | autosomal | (Last reviewed: | provided | | |
| 43939642) | STRC | | recessive | Nov. 1, 2018) | | | |
| | | | 16 | | | | |
| GRCh37/hg19 | CKMT1B, | None | Deafness, | Pathogenic | Not | CV000625827 | 625827 |
| 15q15.3(chr15: 43891364- | CATSPER2, | | autosomal | (Last reviewed: | provided | | |
| 43939659) | STRC | | recessive | Nov. 1, 2018) | | | |
| | | | 16 | | | | |
| GRCh37/hg19 | CATSPER2, | None | Not | Pathogenic | Not | VCV000602122 | 602122 |
| 15q15.3(chr15: 43892807- | STRC | | provided | (Last reviewed: | provided | | |
| 43940669) × 1 | | | | Jul. 18, 2016) | | | |
| NC_000015.9: g.43890 | CKMT1B, | None | Deafness- | Pathogenic | Not | CV000598749 | 598749 |
| 409_43939642del49234 | CATSPER2, | | infertility | (Last reviewed: | provided | | |
| | STRC | | syndrome | Nov. 14, 2017) | | | |
| NM_153700.2: | STRC | None | Deafness, | Pathogenic | Not | none | 692158 |
| c.3499_4701 + 1del | | | autosomal | (Last reviewed: | provided | | |
| | | | recessive | Jul. 29, 2019) | | | |
| | | | 16 | | | | |
| NM_153700.2(STRC): | STRC | None | Rare | Pathogenic | Not | CV000666998 | 666998 |
| c.259C > T | | | genetic | (Last reviewed: | provided | | |
| | | | deafness | Feb. 28, 2019) | | | |
| NM_153700.2(STRC): | STRC | None | Rare | Pathogenic | Not | CV000666997 | 666997 |
| c.4375 + 1G > A | | | genetic | (Last reviewed: | provided | | |
| | | | deafness | Aug. 22, 2018) | | | |
| 15q15.3 deletion | STRC | None | Deafness, | Pathogenic | Not | CV000236065 | 236035 |
| | | | autosomal | (Last reviewed: | provided | | |
| | | | dominant | Feb. 19, 2016) | | | |
| | | | 16 | | | | |

U.S. Application Publication No. 2013/0095071, incorporated by reference herein in its entirety, describes gene therapy methods for restoring age-related hearing loss using mutated tyrosine adeno-associated viral vectors to deliver the X-linked inhibitor of apoptosis protein (XIAP) to the round window membrane of the inner ear. However, the publication does not contemplate the delivery of a nucleic acid sequence encoding functional STRC to prevent or delay the onset of or restore hearing loss caused by genetic mutation of the STRC gene, as disclosed herein.

Additionally, an important pitfall in the current state of the art for developing clinical gene therapies for hearing disorders is a lack of animal models that mirror human hearing loss. Many of the available mouse models for genetic hearing losses with adult onset in humans present with congenital hearing loss making delivery studies complex. There are few models with onset of genetic hearing loss after development of hearing. Delivery of vectors in neonatal mice results in different transfection patterns than delivery in adult mice (Shu, Tao, Li, et al., 2016). There is a need for novel animal models that can be used to evaluate rescue of hearing using different vector systems and gene targets.

There are currently no approved therapeutic treatments for preventing or treating hearing loss or deafness and there is a lack of useful preclinical animal models for testing such treatments. The present invention describes compositions and methods for viral vector gene delivery of STRC into the inner ear to restore activity of a mutated STRC gene, promote hair cell survival and restore hearing in patients suffering from hearing loss or deafness, and cell-based and animal-based models for testing such compositions and methods.

Hearing loss caused by STRC mutations generally presents in two populations: (i) the congenital population where subjects are born with hearing loss and (ii) the progressive population where subjects do not have measurable hearing loss at birth but exhibit progressive hearing loss over a period of time. Therefore, in some instances, a subject may have a mutation in the STRC gene (for example, as detected in a genetic diagnostic test) but does not yet exhibit clinical indicators or symptoms of hearing loss, thus providing a window during which therapeutic intervention can be initiated. Accordingly, in some embodiments, the present invention provides methods for therapeutic intervention during the period of gradual regression of hearing. The methods of the present invention can be commenced prior to such time period. The methods of treating hearing loss provided by the invention include, but are not limited to, methods for preventing or delaying the onset of hearing loss or the progression of clinical indicators or symptoms of hearing loss.

As used herein, the term “hearing loss” is used to describe the reduced ability to hear sound, and includes deafness and the complete inability to hear sound.

The terms “effective amount” or “therapeutically effective amount,” as used herein, refer to an amount of an active agent as described herein that is sufficient to achieve, or contribute towards achieving, one or more desirable clinical outcomes, such as those described in the “treatment” description above. An appropriate “effective” amount in any individual case may be determined using standard techniques known in the art, such as a dose escalation study.

The term “active agent” as used herein refers to a molecule (for example, a Lenti or AAV derived vector as described herein) that is intended to be used in the compositions and methods described herein and that is intended to be biologically active, for example for the purpose of treating hearing loss.

The term “pharmaceutical composition” as used herein refers to a composition comprising at least one active agent as described herein or a combination of two or more active agents, and one or more other components suitable for use in pharmaceutical delivery such as carriers, stabilizers, diluents, dispersing agents, suspending agents, thickening agents, excipients, and the like.

The terms “subject” or “patient” as used interchangeably herein encompass mammals, including, but not limited to, humans, non-human primates, rodents (such as rats, mice and guinea pigs), and the like. In some embodiments of the invention, the subject is a human.

The dose of an active agent of the invention may be calculated based on studies in humans or other mammals carried out to determine efficacy and/or effective amounts of the active agent. The dose amount and frequency or timing of administration may be determined by methods known in the art and may depend on factors such as pharmaceutical form of the active agent, route of administration, whether only one active agent is used or multiple active agents (for example, the dosage of a first active agent required may be lower when such agent is used in combination with a second active agent), and patient characteristics including age, body weight or the presence of any medical conditions affecting drug metabolism.

In one embodiment, a single dose may be administered. In another embodiment, multiple doses may be administered over a period of time, for example, at specified intervals, such as, four times per day, twice per day, once a day, weekly, monthly, and the like.

Clinical characteristics of hearing loss. Hereditary hearing loss and deafness may be conductive, sensorineural, or a combination of both; syndromic (associated with malformations of the external ear or other organs or with medical problems involving other organ systems) or nonsyndromic (no associated visible abnormalities of the external ear or any related medical problems); and prelingual (before language develops) or postlingual (after language develops). (Richard J H Smith, MD, et al.; Deafness and Hereditary Hearing Loss Overview; GeneReviews; Initial Posting: Feb. 14, 1999; Last Revision: Jan. 9, 2014.) Diagnosis/testing. Genetic forms of hearing loss should be distinguished from acquired (non-genetic) causes of hearing loss. The genetic forms of hearing loss are diagnosed by otologic, audiologic, and physical examination, family history, ancillary testing (e.g., CT examination of the temporal bone), and molecular genetic testing. Molecular genetic testing, possible for many types of syndromic and nonsyndromic deafness, plays a prominent role in diagnosis and genetic counseling.

### Selected Tests Used to Measure Hearing Loss:

1. Distortion Product Otoacoustic Emissions (DPOAE). Distortion product otoacoustic emissions (DPOAE) are responses generated when the cochlea is stimulated simultaneously by two pure tone frequencies whose ratio is between 1.1 to 1.3. Recent studies on the generation mechanism of DPOAEs have underlined the presence of two important components in the DPOAE response, one generated by an intermodulation “distortion” and one generated by a “reflection”.

The prevalence of DPOAEs is 100% in normal adult ears. Responses from the left and right ears are often correlated (that is, they are very similar). For normal subjects, women have higher amplitude DPOAEs. Aging processes have an effect on DPOAE responses by lowering the DPOAE amplitude and narrowing the DPOAE response spectrum (i.e. responses at higher frequencies are gradually diminishing). The DPOAEs can be also recorded from other animal species used in clinical research such as lizards, mice, rats, guinea pigs, chinchilla, chicken, dogs and monkeys. (Otoacoustic Emissions Website).

2. Auditory Brainstem Response (ABR). The auditory brainstem response (ABR) test gives information about the inner ear (cochlea) and brain pathways for hearing. This test is also sometimes referred to as auditory evoked potential (AEP). The test can be used with children or others who have a difficult time with conventional behavioral methods of hearing screening. The ABR can also measure WAVE 1 Amplitudes, which is a measure of neuronal activity including the synchronous firing of numerous auditory nerve fibers in the Spiral Ganglion cells (Verhulst, 2016). The ABR is also indicated for a person with signs, symptoms, or complaints suggesting a type of hearing loss in the brain or a brain pathway. The test is used on both humans and animals. The ABR is performed by pasting electrodes on the head—similar to electrodes placed around the heart when an electrocardiogram is run—and recording brain wave activity in response to sound. The person being tested rests quietly or sleeps while the test is performed. No response is necessary. ABR can also be used as a screening test in newborn hearing screening programs. When used as a screening test, only one intensity or loudness level is checked, and the baby either passes or fails the screen. (American Speech-Language-Hearing Association Website).

Clinical Manifestations of hearing loss. Hearing loss is described by type and onset:

### Type

Conductive hearing loss results from abnormalities of the external ear and/or the ossicles of the middle ear.
Sensorineural hearing loss results from malfunction of inner ear structures (i.e., cochlea).
Mixed hearing loss is a combination of conductive and sensorineural hearing loss.
Central auditory dysfunction results from damage or dysfunction at the level of the eighth cranial nerve, auditory brain stem, or cerebral cortex.

### Onset

Prelingual hearing loss is present before speech develops. All congenital (present at birth) hearing loss is prelingual, but not all prelingual hearing loss is congenital.
Postlingual hearing loss occurs after the development of normal speech.

(Richard J H Smith, MD, et al.; Deafness and Hereditary Hearing Loss Overview; GeneReviews; Initial Posting: Feb. 14, 1999; Last Revision: Jan. 9, 2014.)

Severity of hearing loss. Hearing is measured in decibels (dB). The threshold or 0 dB mark for each frequency refers to the level at which normal young adults perceive a tone burst 50% of the time. Hearing is considered normal if an individual's thresholds are within 15 dB of normal thresholds. Severity of hearing loss is graded as shown in Table 6.

TABLE 6
Severity of Hearing Loss in Decibels (dB)

| Severity | Hearing Threshold in Decibels | |
|---|---|---|
| Mild | 26-40 | dB |
| Moderate | 41-55 | dB |
| Moderate Severe | 56-70 | dB |
| Severe | 71-90 | dB |
| Profound | 90 | dB |

Percent hearing impairment. To calculate the percent hearing impairment, 25 dB is subtracted from the pure tone average of 500 Hz, 1000 Hz, 2000 Hz, 3000 Hz. The result is multiplied by 1.5 to obtain an ear-specific level. Impairment is determined by weighting the better ear five times the poorer ear, as shown in Table 7. Because conversational speech is at approximately 50-60 dB HL (hearing level), calculating functional impairment based on pure tone averages can be misleading. For example, a 45-dB hearing loss is functionally much more significant than 30% implies. A different rating scale is appropriate for young children, for whom even limited hearing loss can have a great impact on language development [Northern & Downs 2002].

TABLE 7
Percent Hearing Impairment

| % Impairment | Pure Tone Average (dB)* | % Residual Hearing |
|---|---|---|
| 100% | 91 dB | 0% |
| 80% | 78 dB | 20% |
| 60% | 65 dB | 40% |
| 30% | 45 dB | 70% |

*Pure tone average of 500 Hz, 1000 Hz, 2000 Hz, 3000 Hz

### Frequency of Hearing Loss

The frequency of hearing loss is designated as:
Low (<500 Hz)
Middle (501-2000 Hz)
High (>2000 Hz)

### Gene Therapy

Gene therapy is when DNA is introduced into a patient to treat a genetic disease. The new DNA usually contains a functioning gene to correct the effects of a disease-causing mutation in the existing gene. Gene transfer, either for experimental or therapeutic purposes, relies upon a vector or vector system to shuttle genetic information into target cells. The vector or vector system is considered the major determinant of efficiency, specificity, host response, pharmacology, and longevity of the gene transfer reaction. Currently, the most efficient and effective way to accomplish gene transfer is through the use of vectors or vector systems based on viruses that have been made replication-defective (PCT Publication No. WO 2015/054653; Methods of Predicting Ancestral Virus Sequences and Uses Thereof).

The sensory cells of the adult mammalian cochlea lack the capacity for self-repair; consequently, current therapeutic strategies rely on sound amplification (e.g., hearing aids), better transmission of sound (e.g., middle ear prostheses/active implants), or direct neuronal stimulation (e.g., cochlear implants) to compensate for permanent damage to primary sensory hair cells or spiral ganglion neurons which form the auditory nerve and relay acoustic information to the brain. While these approaches have been transformative, they are not optimal for restoring complex human hearing function important for modern life.

Therapeutic gene transfer to the cochlea has been considered to further improve upon the current standard of care ranging from age-related and environmentally induced hearing loss to genetic forms of deafness such as STRC. More than 300 genetic loci have been linked to hereditary hearing loss with over 70 causative genes described (see e.g., Parker & Bitner-Glindzicz, 2015, Arch. Dis. Childhood, 100:271-8). Therapeutic success in these approaches relies significantly on the safe and efficient delivery of exogenous gene constructs to the relevant therapeutic cell targets in the organ of Corti (OC) in the cochlea.

Conventional viral and non-viral based gene transfer methods can be used to introduce nucleic acids in mammalian cells or target tissues such as the cochlea. Such methods can be used to administer nucleic acids encoding components of a nucleic acid-targeting system to cells in culture, or in a host organism. Non-viral vector delivery systems include DNA plasmids, RNA (e.g., a transcript of a vector), naked nucleic acid, and nucleic acid complexed with a delivery vehicle, such as a liposome. Viral vector delivery systems include DNA and RNA viruses, which have either episomal or integrated genomes after delivery to the cell. Methods of non-viral delivery of nucleic acids include lipofection, nucleofection, microinjection, biolistics, virosomes, liposomes, immunoliposomes, poly cation or lipid:nucleic acid conjugates, naked DNA, artificial virions, and agent-enhanced uptake of DNA. (see e.g., Publication No. JP2022/000041A; Systems, methods and compositions for targeted nucleic acid editing).

### Vectors

To date, adenovirus, adeno-associated virus, herpes simplex virus, vaccinia virus, retrovirus, helper dependent adenovirus and lentivirus have all tested for cochlear gene delivery. Of these, the adeno associated virus (AAV) has demonstrated the most potential but AAV has limited DNA packaging capacity of genes that are less than 4.7 kb in length. The STRC gene is 5.5 kb in length. Two different vector systems will be tested, one based on a lentiviral vector system and the second based on a dual AAV vector system. The Lentiviral vector system disclosed herein has minimal risk of insertional mutagenesis and has been pseudotyped to target hair cells. The lentiviral vector system disclosed herein has been tested in the ear for safety and it has shown consistent delivery to over 95% hair cells from base to apex.

### Lentivirus Vectors

Lentiviruses belong to a genus of the Retroviridae family. They are unique among the retroviruses because they are able to infect mitotic and post-mitotic cells. They can deliver a significant amount of genetic information into the DNA of the host cell, so they are one of the most efficient methods of a gene delivery vector. HIV, SIV, and FIV are all examples of lentiviruses. A lentivirus vector is a vector derived from at least a portion of a lentivirus genome, including especially a self-inactivating lentiviral vector.

Third generation lentiviral vector systems introduced so-called self-inactivating (SIN) vectors. Suitable third generation lentiviral vectors are known in the art and can be prepared and used by the skilled person and are described in, for example, PCT/EP2021/084131, filed Dec. 3, 2021, and incorporated herein by reference in its entirety for all purposes.

An optimal way to achieve replication incompetence is to establish a split packaging design and self-inactivation (SIN) due to a deletion in the U3 region of the 3′ LTR. The genes vif vpr, vpu, nef and, optionally, tat should be eliminated. Specifically, enhancements to the lentiviral system include a 5′ LTR comprising a constitutively active heterologous promoter at the U3 position, a repeat region (R) and a U5 region, a 5′ UTR comprising a primer binding site (PBS), a splice donor site (SD), a packaging signal (ψ), a Rev-responsive element, and, optionally, a splice acceptor (SA) site, an internal enhancer/promoter region operably linked to a cargo sequence, RNA processing elements optionally comprising a Woodchuck hepatitis virus posttranscriptional regulatory element (PRE), and a 3′ LTR with a deleted (SIN) U3 region, a repeat region (R) and a U5 region.

These modifications pseudotype the lentiviral vector for the ability to carry foreign viral envelope proteins on their surface. These viral surface glycoproteins modulate viral entry into the host cell by interacting with particular cellular receptors to induce membrane fusion and make it possible to deliver a cargo load (i.e. STRC) into the inner ear of a subject. Specific enhancements make it possible to pseudotype the lentiviral vector with a viral envelope glycoprotein capable of binding the LDL receptor or LDL-R family members such as MARAV-G, COCV-G, VSV-G or VSV-G ts, and also the SLC1A5-receptor, the Pit1/2-receptor and the PIRYV-G-receptor.

An exemplary lentiviral vector that can be used according to the techniques herein is the first lentiviral sequence disclosed in PCT/EP2021/084131 either partially or in its entirety. The lentiviral vector may also comprise a nucleic acid sequence having at least 80%, at least 85%, at least 90%, at least 95%, at least 96%, at least 97%, at least 98%, or at least 99% sequence identity to the first lentiviral sequence disclosed in PCT/EP2021/084131. It may also consist of the first lentiviral sequence disclosed in PCT/EP2021/084131 in its entirety. Alternatively, if the lentiviral vector is pseudotyped with wild-type VSG, VSV-G or a VSG derivative capable of binding to the LDL-receptor or LDL-R family members, and if the wild type VSV-G is a glycoprotein derived from the Indiana VSV serotype, it may have an amino acid sequence having at least 80%, preferably at least 85%, at least 90%, at least 95%, at least 96%, at least 97%, at least 98%, at least 99% or 100% sequence identity to any of the lentiviral sequences disclosed in PCT/EP2021/084131. To achieve higher particle stability upon in-vivo administration and to evade potential recognition by the host's complement system, a thermostable and complement-resistant VSV-G glycoprotein (VSV-G ts) may alternatively be used, and be capable of binding to the LDL-R or LDL-R family members.

The lentiviral vector may be pseudotyped with a COCV-G glycoprotein, i.e., a glycoprotein derived from Cocal virus. COCV-G is capable of binding to the LDL-receptor. Alternatively, the glycoprotein used for pseudotyping the lentiviral vector of the invention capable of binding to the LDL-receptor is MARAV-G. The lentiviral vector may also be pseudotyped with a viral envelope glycoprotein derived from RD114 glycoprotein (GP) that is capable of binding the SLC1A5-receptor. It may also be a glycoprotein derived from BaEV GP that is capable of binding the SLC1A5-receptor.

The lentiviral vector may also be pseudotyped with a viral envelope glycoprotein capable of binding the Pit1/2-receptor. Pit1 and Pit2 are sodium-dependent phosphate transporters that play a vital role in phosphate transport to ensure normal cellular function. Pit1 and Pit2 serve also as receptors for the gibbon ape leukemia virus (GALV) and the amphotropic murine leukemia virus (A-MuLV), respectively. Therefore, the viral envelope glycoprotein may be derived from GALV. GALV GP is capable of binding the Pit1/2-receptor. Alternatively, the viral glycoprotein may be derived from A-MuLV/Ampho. Such an Ampho GP is capable of binding the Pit1/2-receptor. It may also be pseudotyped with a glycoprotein capable of binding the Pit1/2-receptor and derived from 10A1 MLV.

The lentiviral vector may also be pseudotyped with a glycoprotein capable of binding the Pit1/2-receptor and derived from 10A1 MLV. The lentiviral vector may be alternatively pseudotyped with PIRYV-G. The glycoprotein is thus capable of mediating entry into a host cell that can be entered by PIRYV-G.

At least four different expression plasmids are provided in a process that packages the lentiviral vector. The lentiviral particles may be provided from a vector plasmid encoding the lentiviral vector genome itself as described above, a packaging plasmid coding for Gag and Pol, a plasmid encoding Rev and a plasmid encoding at least one of the herein mentioned envelope glycoproteins. The vector plasmid, the Rev-encoding plasmid, and or the Env-encoding plasmid may be a nucleic acid sequence disclosed in PCT/EP2021/084131.

The techniques herein provide third-generation lentivirus vectors as disclosed in PCT/EP2021/084131 that include a nucleotide sequence encoding the stereocilin gene (STRC) gene operatively connected to a promoter able to drive high levels of STRC expression in the ear cells that express STRC. In some embodiments, the nucleotide sequence encoding STRC may be 95%, 96%, 97%, 98%, 99%, or 100% identical to SEQ ID NO:1. In some embodiments, the promoter may be the human Myo7a promoter or the mouse Myo7a promoter. In some embodiments, the promoter may be 95%, 96%, 97%, 98%, 99%, or 100% identical to SEQ ID NO:4 or SEQ ID NO:6. In some embodiments, the promoter may be 95%, 96%, 97%, 98%, 99%, or 100% identical to SEQ ID NO:4. One of skill in the art will appreciate that the Myo7a promoter sequences represented by SEQ ID NO:4 or SEQ ID NO:6 may need to be shortened to facilitate the ability of a promoter:STRC recombinant nucleic acid to be incorporated into the packaging limitations of the lentivirus vectors disclosed herein. In particular, it is expressly contemplated within the scope of the disclosure that various derivatives of either SEQ ID NO:4 or SEQ ID NO:6 may be constructed that include deletions of the 5′ end of the specified promoter sequence to facilitate the ability of the Myo7a:STRC recombinant nucleotide to be incorporated to the lentivirus vectors disclosed herein in a manner that allows sufficient packaging of the resulting LV-SIN vector into virus particles.

The Myo7a promoter has been characterized, and the core promoter (e.g., SEQ ID NO: 4) is known to be positively regulated by an enhancer located in the first intron of the Myo7a gene (see e.g., Street et al. (2011) A DNA Variant within the MYO7A Promoter Regulates YY1 Transcription Factor Binding and Gene Expression Serving as a Potential Dominant DFNA11 Auditory Genetic Modifier, JBC, 286(17): 15278-15286; Boeda et al. (2001) A specific promoter of the sensory cells of the inner ear defined by trans-Genesis, Human Molecular Genetics, 10(15): 1581-1589), and the human version of the sequences represented by SEQ ID NO:5. It is specifically contemplated within the scope of the disclosure some, or all, portions of the nucleic acid sequence represented by SEQ ID NO:5 may be used in combination with the disclosed promoter sequences in order to facilitate transcriptional activation of STRC. In some embodiments, the enhancer may be 95%, 96%, 97%, 98%, 99%, or 100% identical to SEQ ID NO:5. In some embodiments, SEQ ID NO:4 or SEQ ID NO:6 may be combined with some or all of SEQ ID NO:5 to create a promoter/enhancer combination which may then be operatively linked to STRC and incorporated into a third-generation lentivirus vector disclosed herein. Without being bound be theory, it is believed that such promoter/enhancer combinations may further increase transcriptional activity of STRC in vivo, thereby improving the ability of LV-SIN vectors disclosed herein to rescue STRC⁻ phenotypes in patients having disorders associated with STRC mutations.

### Adeno Associated Virus Vectors

Adeno-associated virus (AAV) vectors are the leading platform for gene delivery for the treatment of a variety of human diseases. Recent advances in developing clinically desirable AAV capsids, optimizing genome designs harnessing revolutionary biotechnologies have contributed substantially to the growth of the gene therapy field. Preclinical and clinical successes in AAV-mediated gene replacement, gene editing and gene silencing have helped AAV become the primary choice for the ideal therapeutic vector, with two AAV-based therapeutics gaining regulatory approval in Europe or the United States (see e.g., Wang, D., Tai, P. W. L. & Gao, G. Adeno-associated virus vector as a platform for gene therapy delivery. (2019) Nat Rev Drug Discov 18, 358-378). Continued study of AAV biology and increased understanding of the associated therapeutic challenges and limitations will build the foundation for future clinical success.

Although adeno-associated viral vector (AAV)-mediated inner ear gene therapy has been applied to animal models of hereditary hearing loss to improve auditory function, infection rates in some cochlear cell types are low. Partly this is due to the large size of AAVs, since only small genes of up to 4.6 kb can be effectively incorporated into the vector without a risk of the production of a truncated protein. In order for inner ear gene therapy to effectively treat hearing loss, a viral vector with higher efficiency is required.

AAV-mediated inner ear gene therapy, delivered into the inner ear involves a precise and focused strategy. The organ of Corti (OC) includes two classes of sensory hair cells: inner hair cells (IHCs), which convert mechanical information carried by sound into electrical signals transmitted to neuronal structures and outer hair cells (OHCs) which serve to amplify and tune the cochlear response, a process required for complex hearing function. Other potential targets in the inner ear include spiral ganglion neurons, columnar cells of the spiral limbus, which are important for the maintenance of the adjacent tectorial membrane or supporting cells, which have protective functions and can be triggered to trans-differentiate into hair cells up to an early neonatal stage.

Injection to the cochlear duct, which is filled with high potassium endolymph fluid, could provide direct access to hair cells. Alterations to this delicate fluid environment, however, may disrupt the endocochlear potential, heightening the risk for injection-related toxicity. Through the oval or round window membrane (RWM), the perilymph-filled spaces surrounding the cochlear duct, scala tympani and scala vestibuli, can be accessed from the middle ear. The RWM, which is the only non-bony opening into the inner ear, is relatively easily accessible in many animal models and administration of viral vector using this route is well tolerated. Cochlear implant placement in humans routinely relies on surgical electrode insertion through the RWM.

Partial rescue of hearing in mouse models of inherited deafness has been a result of previous studies evaluating AAV serotypes in organotypic cochlear explant and in vivo inner ear injection. In these studies, it has been observed that an adeno-associated virus (AAV) containing an ancestral AAV capsid protein transduces OHCs with high efficiency. This finding overcomes the low transduction rates that have limited successful development of cochlear gene therapy using conventional AAV serotypes. An AAV containing an ancestral AAV capsid protein may provide a valuable platform for inner ear gene delivery to IHCs and OHCs, as well as an array of other inner ear cell types that are compromised by genetic hearing and balance disorders. In addition to providing high transduction rates, an AAV containing an ancestral AAV capsid protein was shown to have an analogous safety profile in mouse and nonhuman primate upon systemic injection, and is antigenically distinct from circulating AAVs, providing a potential benefit in terms of pre-existing immunity that limits the efficacy of conventional AAV vectors.

The viruses described herein that contain an ancestral AAV capsid protein can be used to deliver a variety of nucleic acids to inner ear cells. Representative transgenes that can be delivered to, and expressed in, inner ear cells include, without limitation, a transgene that encodes a neurotrophic factor (e.g., glial cell line-derived neurotrophic factor (GDNF), brain-derived neurotrophic factor (BDNF), neurotrophin-3 (NT3), or heat shock protein (HSP)-70), an immunomodulatory protein or an anti-oncogenic transcript. In addition, representative transgenes that can be delivered to, and expressed in, inner ear cells also include, without limitation, a transgene that encodes an antibody or fragment thereof, an antisense, silencing or long non-coding RNA species, or a genome editing system (e.g., a genetically-modified zine finger nuclease, transcription activator-like effector nucleases (TALENs), or clustered regularly interspaced short palindromic repeats (CRISPRs)). Further, representative transgenes that can be delivered to, and expressed in, inner ear cells include nucleic acid STRC presented herein, but may also include ACTG1, ADCY1, ATOHI, ATP6V1B1, BDNF, BDP1, BSND, DATSPER2, CABP2, CD164, CDCl4A, CDH23, CEACAM16, CHD7, CCDC50, CIB2, CLDN14, CLIC5, CLPP, CLRN1, COCH, COL2A1, COL4A3, COL4A4, COL4A5, COL9A1, COL9A2, COL11A1, COL11A2, CRYM, DCDC2, DFNA5, DFNB31, DFNB59, DIAPH1, EDN3, EDNRB, ELMOD3, EMOD3, EPS8, EPS8L2, ESPN, ESRRB, EYA1, EYA4, FAM65B, FOXI1, GIPC3, GJB2, GJB3, GJB6, GPR98, GRHL2, GPSM2, GRXCR1, GRXCR2, HARS2, HGF, HOMER2, HSD17B4, ILDR1, KARS, KCNE1, KCNJ10, KCNQ1, KCNQ4, KITLG, LARS2, LHFPL5, LOXHDI, LRTOMT, MARVELD2, MCM2, MET, MIR183, MIRN96, MITF, MSRB3, MT-RNR1, MT-TS1, MYH14, MYH9, MYO15A, MYO1A, MYO3A, MYO6, MYO7A, NARS2, NDP, NF2, NT3, OSBPL2, OTOA, OTOF, OTOG, OTOGL, P2RX2, PAX3, PCDH15, PDZD7, PJVK, PNPT1, POLR1D, POLR1C, POU3F4, POU4F3, PRPS1, PTPRQ, RDX, S1PR2, SANS, SEMA3E, SERPINB6, SLC17A8, SLC22A4, SLC26A4, SLC26A5, SIX1, SIX5, SMAC/DIABLO, SNAI2, SOX10, SYNE4, TBC1D24, TCOF1, TECTA, TIMM8A, TJP2, TNC, TMC1, TMC2, TMIE, TMEM132E, TMPRSS3, TRPN, TRIOBP, TSPEAR, USH1C, USH1G, USH2A, USH2D, VLGR1, WFS1, WHRN, and XIAP, optionally included in a third-generation lentiviral vector as disclosed herein.

Induced Pluripotent Stem Cells (iPSCs)

An Induced Pluripotent Stem Cell (IPS or IPSCs) is a stem cell that has been created from an adult cell such as a skin, liver, stomach or other mature cell through the introduction of genes that reprogram the cell and transform it into a cell that has all the characteristics of an embryonic stem cell. The term pluripotent connotes the ability of a cell to give rise to multiple cell types, including all three embryonic lineages forming the body's organs, nervous system, skin, muscle and skeleton.

Autologous induced pluripotent stem cells (iPSCs) theoretically constitute an unlimited cell source for patient-specific cell-based organ repair strategies. Their generation, however, poses technical and manufacturing challenges and is a lengthy process that conceptually prevents any acute treatment modalities. Allogeneic iPSC-based therapies or embryonic stem cell-based therapies are easier from a manufacturing standpoint and allow the generation of well-screened, standardized, high-quality cell products. Because of their allogeneic origin, however, such cell products would undergo rejection. With the reduction or elimination of the cells' antigenicity, universally-acceptable cell products could be produced. Because pluripotent stem cells can be differentiated into any cell type of the three germ layers, the potential application of stem cell therapy is wide-ranging. Differentiation can be performed ex vivo or in vivo by transplanting progenitor cells that continue to differentiate and mature in the organ environment of the implantation site. Ex vivo differentiation allows researchers or clinicians to closely monitor the procedure and ensures that the proper population of cells is generated prior to transplantation.

In most cases, however, undifferentiated pluripotent stem cells are avoided in clinical transplant therapies due to their propensity to form teratomas. Rather, such therapies tend to use differentiated cells (e.g., stem cell-derived cardiomyocytes transplanted into the myocardium of patients suffering from heart failure). Clinical applications of such pluripotent cells or tissues would benefit from a “safety feature” that controls the growth and survival of cells after their transplantation.

Pluripotent stem cells (PSCs) may be used because they rapidly propagate and differentiate into many possible cell types. The family of PSCs includes several members generated via different techniques and possessing distinct immunogenic features. Patient compatibility with engineered cells or tissues derived from PSCs determines the risk of immune rejection and the requirement for immunosuppression.

To circumvent the problem of rejection, different techniques for the generation of patient-specific pluripotent stem cells have been developed. These include the transfer of a somatic cell nucleus into an enucleated oocyte (somatic cell nucleus transfer (SCNT) stem cells), the fusion of a somatic cell with an ESC (hybrid cell), and the reprogramming of somatic cells using certain transcription factors (induced PSCs or iPSCs). SCNT stem cells and iPSCs, however, may have immune incompatibilities with the nucleus or cell donor, respectively, despite chromosomal identity. SCNT stem cells carry mitochondrial DNA (mtDNA) passed along from the oocyte. mtDNA-coded proteins can act as relevant minor antigens and trigger rejection. DNA and mtDNA mutations and genetic instability associated with reprogramming and culture-expansion of iPSCs can also create minor antigens relevant for immune rejection. This hurdle decreases the likelihood of successful, large-scale engineering of compatible patient-specific tissues using SCNT stem cells or iPSCs.

### CRISPR/Cas9 Gene Editing

The methods described herein also contemplate the use of CRISPR/Cas9 (clustered regularly interspaced short-palindromic repeats and CRISPR-associated proteins) genome editing to rescue hearing by editing the STRC gene mutation.

This technology has been used to successfully rescue hearing in two genetic hearing loss mouse models (Tmc1 and Pmca2) (Askew, C et al., Tmc gene therapy restores auditory function in deaf mice; Sci Transl Med. 2015 Jul. 8; 7(295):295ra108). While the technology has primarily been used to target dominant hearing loss, it can be developed to target recessive hearing loss and restore hearing in the STRC knock-in mouse model, and ultimately in humans with hearing loss caused by a mutation in the STRC gene. The use of CRISPR/Cas9 gene editing to repair defective gene sequences is further described in PCT Publication No. WO 2016/069910, PCT Publication No. WO 2015/048577, and U.S. Application Publication No. 2015/0291966, each of which are incorporated by reference herein in its entirety.

Conventional molecular biology, microbiology, biochemical, and recombinant DNA techniques within the skill of the art can be used in accordance with the present disclosure. Such techniques are explained fully in the literature and are exemplified in the Examples below. The invention will be further described in the following examples, which do not limit the scope of the methods and compositions of matter described in the claims.

### EXAMPLES

### Example 1: Development of a STRC-Mutant Mouse Model

The development of a mouse model that resembles the human condition as closely as possible is important for initial clinical development. A knock-out STRC mouse model is available from commercial vendors and may be used in the experiments described in these Examples. Additionally, a mouse model that harbors a human a mutation known to cause hearing loss has also been generated using CRISPR/Cas9 technology. The STRC⁻ mouse model shows that the human mutation causes hearing loss in mouse, which makes the model valuable for assessment of the below-described gene therapy constructs.

The disclosure provides a STRC⁻ mouse model carrying a human mutation for the present study. The STRC knock-in mouse model disclosed herein provides the ability to study survival of hair cells and hearing loss by ABR, DPOAE, and histology. Characterization of the mouse is confirming whether the STRC⁻ mouse exhibits the full spectrum of human STRC⁻ phenotypes including: progressive hearing loss, deterioration of stereocilia tip-links, and detachment of stereocilia to the tectorial membrane, which will demonstrate the generation of a STRC mouse model for human DFNB16.

### Example 2: Production of Lentiviral-STRC Constructs for Gene Therapy

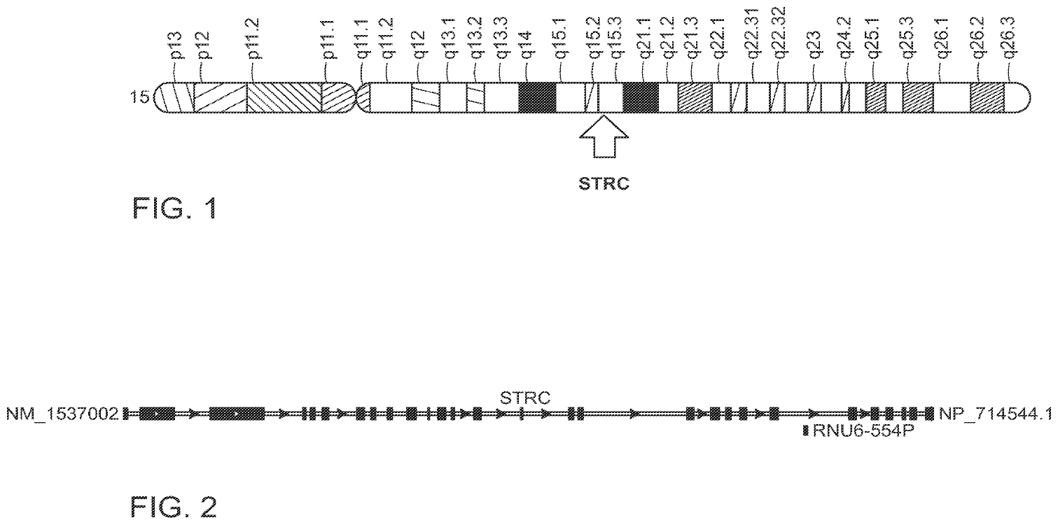

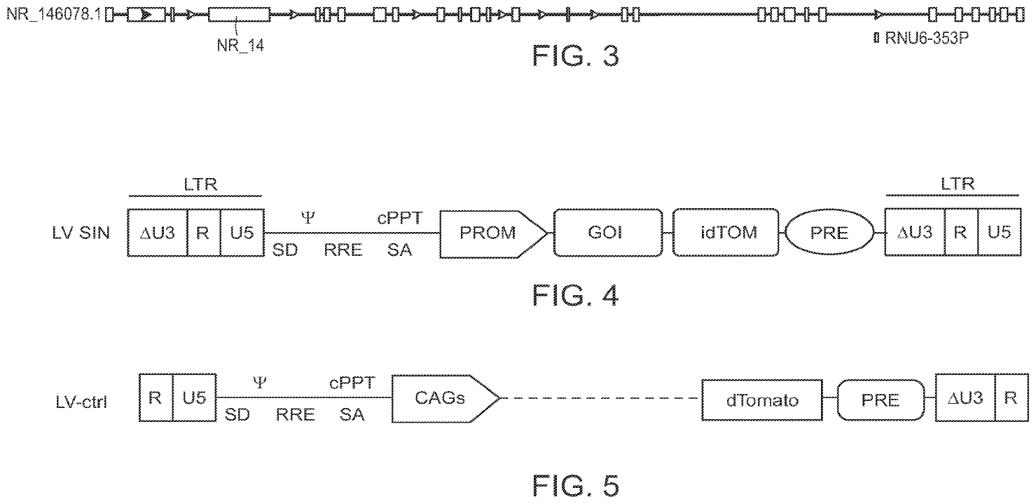

As shown in FIG. 1, the stereocilin (STRC) gene is located on chromosome 15 at position 15q13-q21. FIG. 2 shows the mRNA transcription map of STRC. FIG. 3 shows the mRNA transcription map of a STRC pseudogene.

A novel third-generation, high-capacity lentiviral vector system was used to deliver the large 5,515 bp STRC cDNA plus a dTomato reporter gene in one vector. Briefly, the human STRC cDNA sequence (STRC) as deposited in NCBI (NM_153700) was flanked by a 5′ Kozak consensus sequence and SgrAI/AgeI restriction sites as well as a 3′ SalI restriction site by PCR. The STRC sequence was cloned into a state-of-the-art 3rd generation, self-inactivating (SIN) lentiviral vector harboring a Myo7a promoter resulting in LV-SIN (shown in FIG. 4).

FIG. 4 shows a schematic of a general third generation lentiviral vector including a gene of interest (GOI) and a promoter (PROM), where the GOI is STRC and the promoter is Myo7a (e.g., SEQ ID NO: 4 or SEQ ID NO: 6).

A control vector only expressing the dTomato reporter driven by an SFFV promoter was generated by inserting the dTomato sequence flanked by AgeI and SalI into the vector backbone using the unique AgeI and SalI restriction sites, generating pRRL.PPT.SF.dTomato.pre (LV-ctrl) as shown in FIG. 5.

In order to establish a gene therapeutic option for STRC mutations, a high-capacity 3^{rd} generation lentiviral vector was equipped with the large 5,515 bp cDNA sequence of the native STRC isoform. The vector harbored a self-inactivating (SIN) architecture devoid of the enhancer and promoter elements naturally present in the long-terminal repeats (LTRs). This design confers an improved safety profile by reducing the risk of insertional mutagenesis, and allows the usage of an internal promoter of choice (e.g., prestin, myosin 6, myosin 7, myosin 15 or hcmv promoters) to drive transgene expression. Here, the myo7a promoter was chosen to mediate high-level and sustained cell-type specific expression of the transgene cassette. To facilitate titration of viral vector particle preparations and identification of successfully transduced cells upon in-vitro and in-vivo application, the STRC cDNA was linked to a dTomato reporter gene via an internal ribosomal entry site (IRES) to create the lentiviral vector LV-SIN; shown in FIG. 4. A counterpart expressing dTomato only served as a reference and control (LV-ctrl) and is shown in FIG. 5.

Transient production using a split-packaging system successfully generated lentiviral particles despite the challenging size of the STRC cDNA. LV titers were in a range that is sufficient for in vitro and in vivo application.

### Example 3: Lentiviral STRC Constructs are Expressed in the Otic Cell Lines and Organ of Corti Cultures

The ability of LV-SIN to drive STRC expression was initially tested in HEI-OC1 Otic cell lines. MYO7A and dTomato were successfully expressed upon in-vitro transduction of the cochlea-derived cell line HEI-OC1, which is one of the few mouse auditory cell lines available for research purposes. HEI-OC1 cells are useful for investigating drug-activated apoptotic pathways, autophagy, senescence, mechanisms of cell protection, inflammatory responses, cell differentiation, genetic and epigenetic effects of pharmacological drugs, etc. According to the techniques herein, HEI-OC1 cells may be used to assess expression of gene constructs in auditory cells. Importantly, HEI-OC1 cells endogenously express prestin, an important motor protein of outer hair cells. In this regard, HEI-OC1 cells serve as a useful in vitro auditory model.

Evaluating vector functionality and the capacity to transduce inner ear cells, LV-SINLV-SIN was tested for its in vitro performance using the established hair-cell-like cell line HEI-OC1 (Kalinec et al. (2003) A cochlear cell line as an in vitro system for drug ototoxicity screening. Audiol. Neurotol.).

HEI-OC1 cells were seeded at 3×10⁴ per well of a 24-well plate on the day prior to transduction. Three wells were harvested for counting to determine the cell number at the time point of transduction, and the volume of viral vector supernatant was calculated based on the vector's titer to apply defined multiplicities of infection (MOI), i.e. a defined particle number per seeded cell. The transduction procedure followed the same protocol as described under titration. The percentage of cells expressing the vector-encoded dTomato reporter protein was assessed by flow cytometry as described under titration.

Cells were harvested using trypsin-assisted detachment and pelletized by centrifugation for 5 min at 400 xg. The pellets were resuspended in 500 μL Fixation Buffer (Cat #420801, BioLegend, San Diego, CA, USA) and cells incubated for 20 min at room temperature. Samples were pelletized again and washed with 1 mL FACS buffer, followed by three cycles of resuspension in 1× Intracellular Staining Perm Wash Buffer (Cat #421002, BioLegend) and centrifugation for 5 min at 400 xg. Incubation with the primary antibody polyclonal rabbit-anti-myosin-VIIA (Catalog #25-6790, Proteus BioSciences Inc., Ramona, CA, USA) was performed at 1:300 dilution in 1× Intracellular Staining Perm Wash Buffer for 20 min at room temperature, followed by two washes with 1× Intracellular Staining Perm Wash Buffer. Incubation with the secondary antibody Alexa Fluor® 488 AffiniPure Donkey Anti-Rabbit IgG (H+L) (Catalog #711-545-152, Jackson ImmunoResearch Europe Ltd, Ely, UK) was performed at 1:800 dilution in 1× Intracellular Staining Perm Wash Buffer for 20 min at room temperature in the dark. After two washes with 1× Intracellular Staining Perm Wash Buffer, cell pellets were resuspended in FACS buffer, processed on a CytoFLEX S flow cytometer and analyzed using CytExpert software.

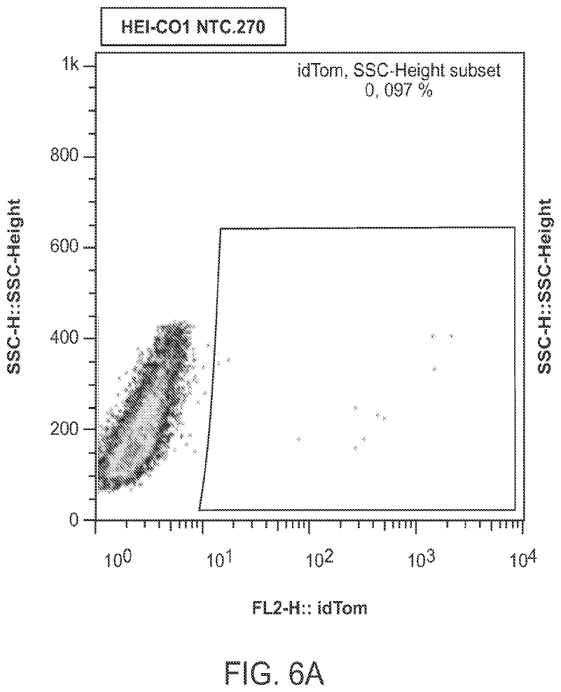

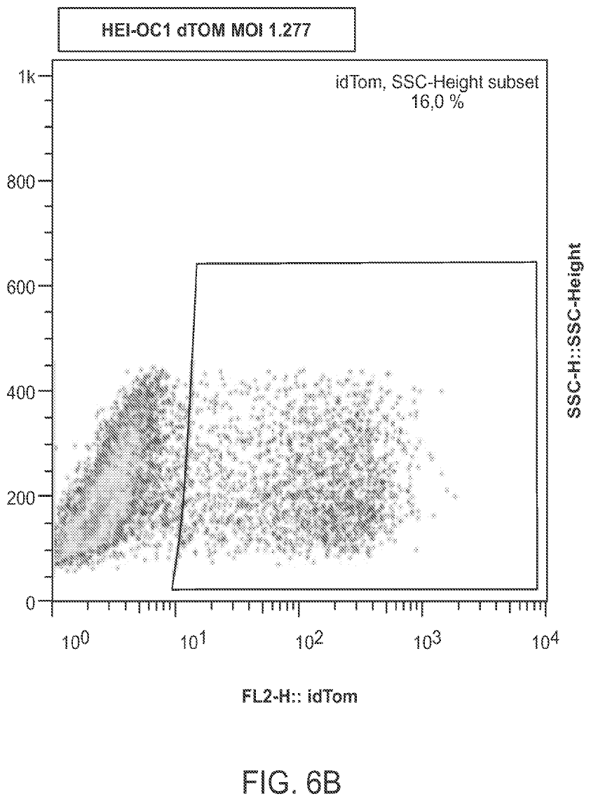

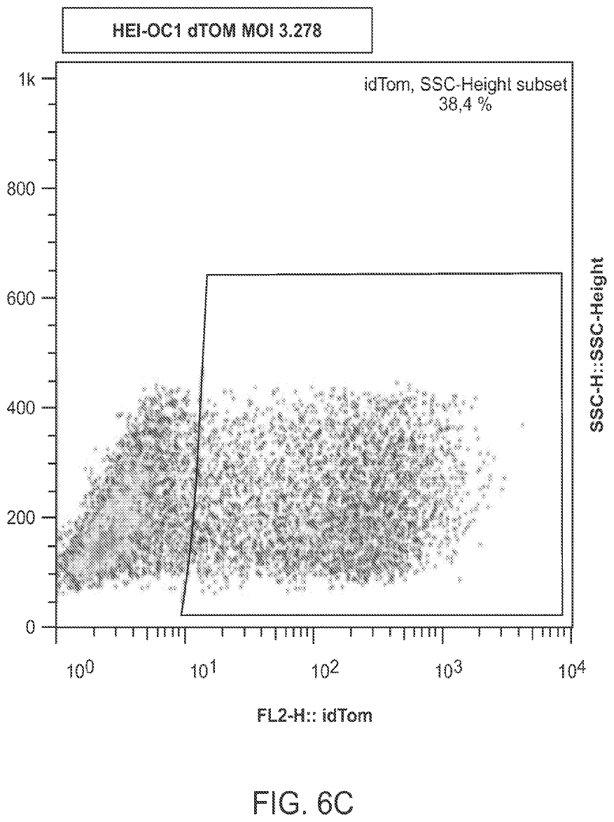

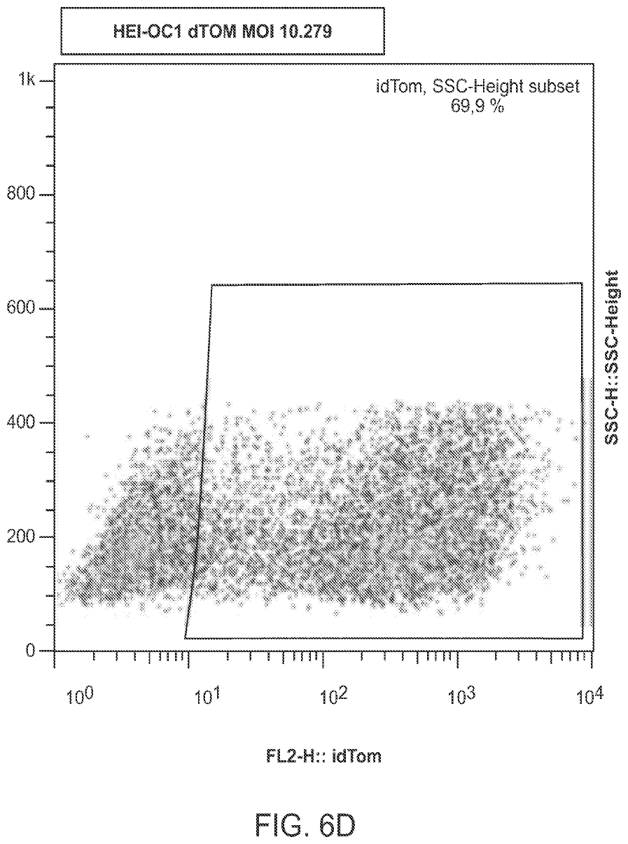

Upon transduction at different multiplicity of infection (MOI), i.e. applying defined numbers of viral vector particles per seeded cell, no significant difference in the percentage of successfully transduced, dTomato-positive cells was observed by flow cytometry analysis between LV-SINLV-SIN and LV-ctrl across all MOIs tested. FIGS. 6A-6D are a series of dotplots showing dTom expression in HEI-OC1 cells. In particular, the percentage of HEI-OC1 cells expressing the vector-encoded dTomato reporter and the STRC protein. Flow cytometry analysis was performed upon intracellular staining for dTom expression in non-transduced controls (NTC) and cells transduced with LV-ctrl or LV-SIN at a series of different MOIs. The populations shown were pre-gated for live cells using SSC-A/FSC-A characteristics, followed by gating for single cells according to FSC-A/FSC-H characteristics. FIG. 6A shows data for NTC. FIG. 6B shows dTom expression at MOI 1.277. FIG. 6C shows dTom expression at MOI 3.278. FIG. 6D shows dTom expression at MOI 10.279. This confirmed that the transduction efficiency of the lentiviral vector encoding the large STRC cDNA was comparable to smaller vectors.

Visualization via immunofluorescence microscopy or flow cytometry revealed low-level endogenous STRC expression in the non-transduced HEI-OC1 cells and no signal for dTomato (FIGS. 6A-6D). Altogether, despite the large size of the STRC transgene, fully functional LV vector particles could be produced that successfully transferred and expressed STRC in otic target cells.

### Example 4: Lentiviral STRC Constructs are Expressed in the Inner Ear of the Mouse

Having confirmed that STRC can be delivered by and expressed from LV-STRC, the ability of STRC to be expressed appropriately in vivo was investigated. Adult C57BL/6 mice aged 16 days were anesthetized with an intraperitoneal (IP) injection of a mixture of ketamine (150 mg/kg), xylocaine (6 mg/kg) and acepromazine (2 mg/kg) in sodium chloride 0.9%. A dorsal postauricular incision was made, and the posterior semicircular canal exposed. Using a microdrill, a canalostomy was created, exposing the perilymphatic space. Subsequently, 1 μL of vector was injected using a Hamilton microsyringe with 0.1 μL graduations and a 36 gauge needle. The canalostomy was sealed with bone wax, and the animals were allowed to recover.

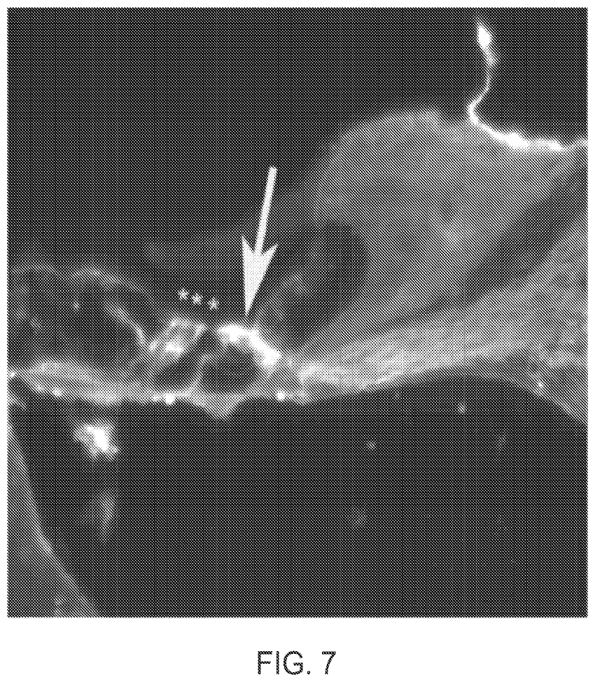

LV-SIN was injected into the inner ear of a wildtype mouse as described above to assess the ability of LV-STRC to drive in vivo expression of human STRC. As shown in FIG. 7, STRC (as visualized by dTom expression) was robustly expressed in the inner ear of the mouse. In particular, robust expression was observed in the inner hair cells (arrow) and outer hair cells (stars) was detected. The characteristics of successful packaging and efficient in vivo delivery of STRC in the absence of adverse effects to wildtype mice indicate LV-SIN to be a suitable candidate for in vivo gene therapy of STRC related genetic disorders.

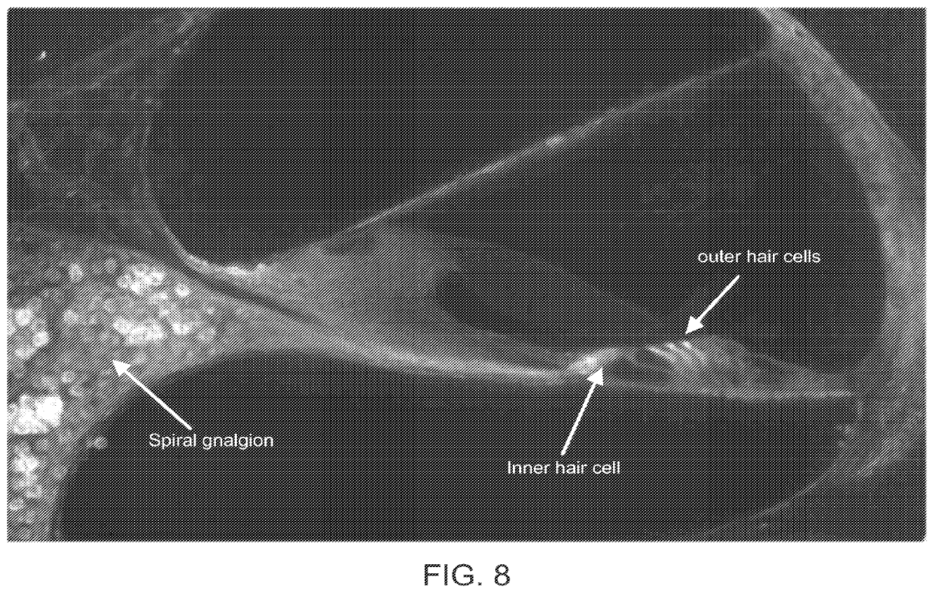

FIG. 8 shows the distribution of pseudotyped LV-hcmv-dTom in the adult mouse inner ear. Delivery of 1×10\hat{ }6 PU to the posterior semicircular canal of a P30 C57Bl/6 mouse. Expression of dTom can be seen in all hair cells as well as in the spiral ganglion demonstrating the capacity of this vector to target the cells targeted by mutations in STRC.

### Example 5: Study of LV-SIN in Restoration of Hearing

LV-SIN is injected into the neonatal STRC⁻ mutant mouse inner ear. Analysis is performed for the injected and control mice injected with LV-GFP/dTom, which may include hearing tests, cellular and molecular studies and long-term effect. LV-SIN may be assessed at the cellular level to determine whether it promotes hair cell survival at one month of age. In control mutant ears injected with LV-GFP/dTom, it is expected that there will be a loss of hair cells at this time point. In contrast, it is expected that LV-SIN injected hair cells will survive. The injection procedure (cochleostomy, round window membrane, canalostomy) and doses for better hearing recovery. Importantly, injections may be performed in adult (1-6 months of age) mice to assess the possibility of hearing recovery. Adult injection results will be compared with neonatal results, which provide information about the time window in which intervention is still effective.

### Example 6: Study of Hair Cells Derived from Patient Induced Pluripotent Stem Cells (iPS) Cells

One important aspect of the study is to demonstrate that the techniques disclosed herein may be effective on human hair cells. As no human temporal bone is available for the study, iPS cell lines are established from patient iPS cells using patient fibroblasts as well as control family member fibroblasts. The fibroblasts are harvested from the patients with the most frequent mutation and the iPS cell lines are established. The iPS cell lines are differentiated into inner ear cells including hair cells. With the culture system, LV-SIN is used to infect iPS-derived hair cells. Infected hair cells are studied for survival and hair cell transduction by patchy clamping. It is expected to see improved hair cell survival and hair cell function, compared to the uninfected and un-treated control hair cells. The study provides the opportunities to evaluate the efficiency of LENTI-STRC infection in human hair cells and expression of STRC gene. Such achievement is a demonstration that defective human hair cells can be treated with LV-SIN, which makes it one major step forward to future clinical studies.

1. A lentivirus expression vector comprising:
a nucleic acid sequence encoding Stereocilin (STRC), or a part thereof; and
a promoter operatively linked to the nucleic acid sequence.

2. The lentivirus expression vector of claim 1, wherein the lentivirus expression vector is a third-generation self-inactivating (SIN) lentivirus vector, optionally wherein the SIN lentivirus vector lacks wildtype lentivirus long-terminal repeat (LTR) enhancer and promoter elements.

3. (canceled)

4. The lentivirus expression vector of claim 1, wherein the promoter is selected from the group consisting of STRC promoters, Myo7a promoters, human cytomegalovirus (HCMV) promoters, cytomegalovirus/chicken beta-actin (CBA) promoters and Pou4f3 promoters, optionally wherein the promoter is Myo7a, optionally further comprising a Myo7a enhancer, optionally wherein the promoter is 95%, 96%, 97%, 98%, 99%, or 100% identical to SEQ ID NO: 4 or SEQ ID NO: 6, optionally further comprising a Myo7a enhancer 95%, 96%, 97%, 98%, 99%, or 100% identical to SEQ ID NO: 5.

5-6. (canceled)

7. The lentivirus expression vector of claim 1, wherein the nucleic acid is 95%, 96%, 97%, 98%, 99%, or 100% identical to SEQ ID NO: 1.

8. The lentivirus expression vector of claim 1, wherein the nucleic acid encodes a polypeptide 95%, 96%, 97%, 98%, 99%, or 100% identical to SEQ ID NO: 2.

9. A pharmaceutical composition for use in a method for the treatment or prevention of hearing loss comprising a lentivirus expression vector comprising a nucleic acid which is 95%, 96%, 97%, 98%, 99%, or 100% identical to SEQ ID NO: 1, wherein the nucleic acid sequence is operatively linked to a nucleic acid which is 95%, 96%, 97%, 98%, 99%, or 100% identical to SEQ ID NO: 4 or SEQ ID NO: 6.

10. A cell comprising a lentivirus expression vector comprising the nucleic acid sequence of SEQ ID NO:1; and a promoter operatively linked to the nucleic acid.

11. The cell of claim 10, wherein the nucleic acid which is 95%, 96%, 97%, 98%, 99%, or 100% identical to SEQ ID NO: 1, or wherein the promoter is selected from the group consisting of STRC promoters, Myo7a promoters, human cytomegalovirus (HCMV) promoters, cytomegalovirus/chicken beta-actin (CBA) promoters or Pou4f3 promoters, optionally wherein the promoter is Myo7a, optionally wherein the promoter is 95%, 96%, 97%, 98%, 99%, or 100% identical to SEQ ID NO: 4 or SEQ ID NO: 6.

12-14. (canceled)

15. The cell of claim 10, wherein the cell is a stem cell.

16. The cell of claim 15, wherein the stem cell is an induced pluripotent stem cell.

17. A method for treating or preventing hearing loss, comprising administering to a subject in need thereof an effective amount of the lentivirus vector of claim 1.

18. The method of claim 17, wherein the promoter is selected from the group consisting of STRC promoters, Myo7a promoters, human cytomegalovirus (HCMV) promoters, cytomegalovirus/chicken beta-actin (CBA) promoters, or Pou4f3 promoters.

19. The method of claim 18, wherein the promoter is Myo7a.

20. The method of claim 19, wherein the promoter is 95%, 96%, 97%, 98%, 99%, or 100% identical to SEQ ID NO: 4 or SEQ ID NO: 6.

21. The method of claim 17, wherein the expression vector is administered by injection into the inner ear of the subject.

22. The method of claim 21, wherein the injection method is selected from the group consisting of cochleostomy, round window membrane, endolymphatic sac, scala media, canalostomy, scala media via the endolymphatic sac, or any combination thereof.

23. The method of claim 17, wherein the subject has one or more genetic risk factors associated with hearing loss.

24. The method of claim 23, wherein one of the genetic risk factors is selected from the group consisting of a mutation in the STRC gene.

25. The method of claim 23, wherein the subject does not exhibit any clinical indicators of hearing loss.

26. A transgenic mouse comprising a mutation/variation that causes hearing loss selected from a group consisting of a mutation/variation in the human STRC gene, wherein the mouse is generated using the lentivirus vector of claim 1.


SEQUENCE LISTING (6 sequences)

SEQ ID NO: 1 - DNA, 5515 bp, human
gccctgccctcacctggctatcccacacaggtgagaataaccagaactcacctccggtaccagtgttcacttggaaacatggctctcagcctctggcccctgctgctgctgctgctgctgctgctgctgctgtcctttgcagtgactctggcccctactgggcctcattccctggaccctggtctctccttcctgaagtcattgctctccactctggaccaggctccccagggctccctgagccgctcacggttctttacattcctggccaacatttcttcttcctttgagcctgggagaatgggggaaggaccagtaggagagcccccacctctccagccgcctgctctgcggctccatgattttctagtgacactgagaggtagccccgactgggagccaatgctagggctgctaggggatatgctggcactgctgggacaggagcagactccccgagatttcctggtgcaccaggcaggggtgctgggtggacttgtggaggtgctgctgggagccttagttcctgggggcccccctaccccaactcggcccccatgcacccgtgatgggccgtctgactgtgtcctggctgctgactggttgccttctctgctgctgttgttagagggcacacgctggcaagctctggtgcaggtgcagcccagtgtggaccccaccaatgccacaggcctcgatgggagggaggcagctcctcactttttgcagggtctgttgggtttgcttaccccaacaggggagctaggctccaaggaggctctttggggcggtctgctacgcacagtgggggcccccctctatgctgcctttcaggaggggctgctccgtgtcactcactccctgcaggatgaggtcttctccattttggggcagccagagcctgataccaatgggcagtgccagggaggtaaccttcaacagctgctcttatggggcgtccggcacaacctttcctgggatgtccaggcgctgggctttctgtctggatcaccacccccaccccctgccctccttcactgcctgagcacgggcgtgcctctgcccagagcttctcagccgtcagcccacatcagcccacgccaacggcgagccatcactgtggaggccctctgtgagaaccacttaggcccagcaccaccctacagcatttccaacttctccatccacttgctctgccagcacaccaagcctgccactccacagccccatcccagcaccactgccatctgccagacagctgtgtggtatgcagtgtcctgggcaccaggtgcccaaggctggctacaggcctgccacgaccagtttcctgatgagtttttggatgcgatctgcagtaacctctccttttcagccctgtctggctccaaccgccgcctggtgaagcggctctgtgctggcctgctcccaccccctaccagctgccctgaaggcctgccccctgttcccctcaccccagacatcttttggggctgcttcttggagaatgagactctgtgggctgagcgactgtgtggggaggcaagtctacaggctgtgccccccagcaaccaggcttgggtccagcatgtgtgccagggccccaccccagatgtcactgcctccccaccatgccacattggaccctgtggggaacgctgcccggatgggggcagcttcctggtgatggtctgtgccaatgacaccatgtatgaggtcctggtgcccttctggccttggctagcaggccaatgcaggataagtcgtgggggcaatgacacttgcttcctagaagggctgctgggcccccttctgccctctctgccaccactgggaccatccccactctgtctgacccctggccccttcctccttggcatgctatcccagttgccacgctgtcagtcctctgtcccagctcttgctcaccccacacgcctacactatctcctccgcctgctgaccttcctcttgggtccaggggctgggggcgctgaggcccaggggatgctgggtcgggccctactgctctccagtctcccagacaactgctccttctgggatgcctttcgcccagagggccggcgcagtgtgctacggacgattggggaatacctggaacaagatgaggagcagccaaccccatcaggctttgaacccactgtcaaccccagctctggtataagcaagatggagctgctggcctgctttagtcctgtgctgtgggatctgctccagagggaaaagagtgtttgggccctgcagattctagtgcaggcgtacctgcatatgcccccagaaaacctccagcagctggtgctttcagcagagagggaggctgcacagggcttcctgacactcatgctgcaggggaagctgcaggggaagctgcaggtaccaccatccgaggagcaggccctgggtcgcctgacagccctgctgctccagcggtacccacgcctcacctcccagctcttcattgacctgtcaccactcatccctttcttggctgtctctgacctgatgcgcttcccaccatccctgttagccaacgacagtgtcctggctgccatccgggattacagcccaggaatgaggcctgaacagaaggaggctctggcaaagcgactgctggcccctgaactgtttggggaagtgcctgcctggccccaggagctgctgtgggcagtgctgcccctgctcccccacctccctctggagaactttttgcagctcagccctcaccagatccaggccctggaggatagctggccagcagcaggtctggggccagggcatgcccgccatgtgctgcgcagcctggtaaaccagagtgtccaggatggtgaggagcaggtacgcaggcttgggcccctcgcctgtttcctgagccctgaggagctgcagagcctagtgcccctgagtgatccaacggggccagtagaacgggggctgctggaatgtgcagccaatgggaccctcagcccagaaggacgggtggcatatgaacttctgggtgtgttgcgctcatctggaggagcggtgctgagcccccgggagctgcgggtctgggcccctctcttctctcagctgggcctccgcttccttcaggagctgtcagagccccagcttagagccatgcttcctgtcctgcagggaactagtgttacacctgctcaggctgtcctgctgcttggacggctccttcctaggcacgatctatccctggaggaactctgctccttgcaccttctgctaccaggcctcagcccccagacactccaggccatccctaggcgagtcctggtcggggcttgttcctgcctggcccctgaactgtcacgcctctcagcctgccagaccgcagcactgctgcagacctttcgggttaaagatggtgttaaaaatatgggtacaacaggtgctggtccagctgtgtgtatccctggtcagcctattcccaccacctggccagactgcctgcttcccctgctcccattaaagctgctacaactggattccttggctcttctggcaaatcgaagacgctactgggagctgccctggtctgagcagcaggcacagtttctctggaagaagatgcaagtacccaccaaccttaccctcaggaatctgcaggctctgggcaccctggcaggaggcatgtcctgtgagtttctgcagcagatcaactccatggtagacttccttgaagtggtgcacatgatctatcagctgcccactagagttcgagggagcctgagggcctgtatctgggcagagctacagcggaggatggcaatgccagaaccagaatggacaactgtagggccagaactgaacgggctggatagcaagctactcctggacttaccgatccagttgatggacagactatccaatgaatccattatgttggtggtggagctggtgcaaagagctccagagcagctgctggcactgacccccctccaccaggcagccctggcagagagggcactacaaaacctggctccaaaggagactccagtctcaggggaagtgctggagaccttaggccctttggttggattcctggggacagagagcacacgacagatccccctacagatcctgctgtcccatctcagtcagctgcaaggcttctgcctaggagagacatttgccacagagctgggatggctgctattgcaggagtctgttcttgggaaaccagagttgtggagccaggatgaagtagagcaagctggacgcctagtattcactctgtctactgaggcaatttccttgatccccagggaggccttgggtccagagaccctggagcggcttctagaaaagcagcagagctgggagcagagcagagttggacagctgtgtagggagccacagcttgctgccaagaaagcagccctggtagcaggggtggtgcgaccagctgctgaggatcttccagaacctgtgccaaattgtgcagatgtacgagggacattcccagcagcctggtctgcaacccagattgcagagatggagctctcagactttgaggactgcctgacattatttgcaggagacccaggacttgggcctgaggaactgcgggcagccatgggcaaagcaaaacagttgtggggtcccccccggggatttcgtcctgagcagatcctgcagcttggtaggctcttaataggtctaggagatcgggaactacaggagctgatcctagtggactggggagtgctgagcaccctggggcagatagatggctggagcaccactcagctccgcattgtggtctccagtttcctacggcagagtggtcggcatgtgagccacctggacttcgttcatctgacagcgctgggttatactctctgtggactgcggccagaggagctccagcacatcagcagttgggagttcagccaagcagctctcttcctcggcaccctgcatctccagtgctctgaggaacaactggaggttctggcccacctacttgtactgcctggtgggtttggcccaatcagtaactgggggcctgagatcttcactgaaattggcaccatagcagctgggatcccagacctggctctttcagcactgctgcggggacagatccagggcgttactcctcttgccatttctgtcatccctcctcctaaatttgctgtggtgtttagtcccatccaactatctagtctcaccagtgctcaggctgtggctgtcactcctgagcaaatggcctttctgagtcctgagcagcgacgagcagttgcatgggcccaacatgagggaaaggagagcccagaacagcaaggtcgaagtacagcctggggcctccaggactggtcacgaccttcctggtccctggtattgactatcagcttccttggccacctgctatgagcctgtctctacagtagaaggagattgtggggagagaaatcttaagtcataatgaataaagtgcaaacagaagtgcatcctgattattttcagaagctgatgaggaata

SEQ ID NO: 2 - protein, 1775 aa, human
MALSLWPLLLLLLLLLLLSFAVTLAPTGPHSLDPGLSFLKSLLSTLDQAPQGSLSRSRFFTFLANISSSFEPGRMGEGPVGEPPPLQPPALRLHDFLVTLRGSPDWEPMLGLLGDMLALLGQEQTPRDFLVHQAGVLGGLVEVLLGALVPGGPPTPTRPPCTRDGPSDCVLAADWLPSLLLLLEGTRWQALVQVQPSVDPTNATGLDGREAAPHFLQGLLGLLTPTGELGSKEALWGGLLRTVGAPLYAAFQEGLLRVTHSLQDEVFSILGQPEPDTNGQCQGGNLQQLLLWGVRHNLSWDVQALGFLSGSPPPPPALLHCLSTGVPLPRASQPSAHISPRQRRAITVEALCENHLGPAPPYSISNFSIHLLCQHTKPATPQPHPSTTAICQTAVWYAVSWAPGAQGWLQACHDQFPDEFLDAICSNLSFSALSGSNRRLVKRLCAGLLPPPTSCPEGLPPVPLTPDIFWGCFLENETLWAERLCGEASLQAVPPSNQAWVQHVCQGPTPDVTASPPCHIGPCGERCPDGGSFLVMVCANDTMYEVLVPFWPWLAGQCRISRGGNDTCFLEGLLGPLLPSLPPLGPSPLCLTPGPFLLGMLSQLPRCQSSVPALAHPTRLHYLLRLLTFLLGPGAGGAEAQGMLGRALLLSSLPDNCSFWDAFRPEGRRSVLRTIGEYLEQDEEQPTPSGFEPTVNPSSGISKMELLACFSPVLWDLLQREKSVWALQILVQAYLHMPPENLQQLVLSAEREAAQGFLTLMLQGKLQGKLQVPPSEEQALGRLTALLLQRYPRLTSQLFIDLSPLIPFLAVSDLMRFPPSLLANDSVLAAIRDYSPGMRPEQKEALAKRLLAPELFGEVPAWPQELLWAVLPLLPHLPLENFLQLSPHQIQALEDSWPAAGLGPGHARHVLRSLVNQSVQDGEEQVRRLGPLACFLSPEELQSLVPLSDPTGPVERGLLECAANGTLSPEGRVAYELLGVLRSSGGAVLSPRELRVWAPLFSQLGLRFLQELSEPQLRAMLPVLQGTSVTPAQAVLLLGRLLPRHDLSLEELCSLHLLLPGLSPQTLQAIPRRVLVGACSCLAPELSRLSACQTAALLQTFRVKDGVKNMGTTGAGPAVCIPGQPIPTTWPDCLLPLLPLKLLQLDSLALLANRRRYWELPWSEQQAQFLWKKMQVPTNLTLRNLQALGTLAGGMSCEFLQQINSMVDFLEVVHMIYQLPTRVRGSLRACIWAELQRRMAMPEPEWTTVGPELNGLDSKLLLDLPIQLMDRLSNESIMLVVELVQRAPEQLLALTPLHQAALAERALQNLAPKETPVSGEVLETLGPLVGFLGTESTRQIPLQILLSHLSQLQGFCLGETFATELGWLLLQESVLGKPELWSQDEVEQAGRLVFTLSTEAISLIPREALGPETLERLLEKQQSWEQSRVGQLCREPQLAAKKAALVAGVVRPAAEDLPEPVPNCADVRGTFPAAWSATQIAEMELSDFEDCLTLFAGDPGLGPEELRAAMGKAKQLWGPPRGFRPEQILQLGRLLIGLGDRELQELILVDWGVLSTLGQIDGWSTTQLRIVVSSFLRQSGRHVSHLDFVHLTALGYTLCGLRPEELQHISSWEFSQAALFLGTLHLQCSEEQLEVLAHLLVLPGGFGPISNWGPEIFTEIGTIAAGIPDLALSALLRGQIQGVTPLAISVIPPPKFAVVFSPIQLSSLTSAQAVAVTPEQMAFLSPEQRRAVAWAQHEGKESPEQQGRSTAWGLQDWSRPSWSLVLTISFLGHLL

SEQ ID NO: 3 - DNA, 19238 bp, human
gccctgccctcacctggctatcccacacaggtgagaataaccagaactcacctccggtaccagtgttcacttggaaacatggctctcagcctctggcccctgctgctgctgctgctgctgctgctgctgctgtcctttgcaggtaagaagaacagtgagcagaactggggatgaggaggagggtggctggaaaaagactttaagaatatggaggtgaacctgttagatagaaggacaaaggagagaggcagagacttgtgcaaaagggaaaaatgagggttaagaaaagcaggccaagacttactgtaggccagtgaaaggggttcagctcaccatcccctcacctcatctttagatccaggtagggaactgtgctcaggggcagggttgagtttgggctctgtgttcctctccttcagtgacctctggtttctctccttacagtgactctggcccctactgggcctcattccctggaccctggtctctccttcctgaagtcattgctctccactctggaccaggctccccagggctccctgagccgctcacggttctttacattcctggccaacatttcttcttcctttgagcctgggagaatgggggaaggaccagtaggagagcccccacctctccagccgcctgctctgcggctccatgattttctagtgacactgagaggtagccccgactgggagccaatgctagggctgctaggggatatgctggcactgctgggacaggagcagactccccgagatttcctggtgcaccaggcaggggtgctgggtggacttgtggaggtgctgctgggagccttagttcctgggggcccccctaccccaactcggcccccatgcacccgtgatgggccgtctgactgtgtcctggctgctgactggttgccttctctgctgctgttgttagagggcacacgctggcaagctctggtgcaggtgcagcccagtgtggaccccaccaatgccacaggcctcgatgggagggaggcagctcctcactttttgcagggtctgttgggtttgcttaccccaacaggggagctaggctccaaggaggctctttggggcggtctgctacgcacagtgggggcccccctctatgctgcctttcaggaggggctgctccgtgtcactcactccctgcaggatgaggtcttctccattttggggcagccagagcctgataccaatgggcagtgccagggaggtgagtgtggccagggctgggactgggatgtggcagggcaaggaaagtgaaattggggtagttttcttccttactctttccctcctaggtaaccttcaacagctgctcttatggtaagtaacaggagaccagttctgagggattgggcctggaaaatctggaggtgaagagctgaagacctcagcctctagagaggaaaactgatgggaggagtgtagtttagtggttttggggtgtgactgtctgggttggtgtcccagctccacctcttcctagccatatgaccttgagcaggttacatagtctttctatacctcagtttccccatttataaaatgagaatgataatattagttaccacagagttgttgcacccggttaaatgagttgatactgtgtatgcaaacgacttaaaaccgtgctggcacatagcgcttaataatgttagctagtaaagatgggatttggaaaataaggacacagctggattcctctacccccttactacttcagtacaacaatgccagacagtagttagacatattgagttgctgagcagatttcctaacatgaggcccgctgagggttgtgtttaagctatctaaaagcatacgaagaaaggagacagaagggggccaggtggacagaaagaattccaactggggcttctcctaggtgattttggaccttggcagggcagctttctcttttttgccccgttgcagcatttcaaccagtaacgcctaaactctcagggacctcgcttgtagaaaagcctatgcttgccatgccccttgagggctctgagtcagggtcagaatcttcagctggaggaaatgtgaactgaccagatcctgcctgctcctccctctgcacccaggggcgtccggcacaacctttcctgggatgtccaggcgctgggctttctgtctggatcaccacccccaccccctgccctccttcactgcctgagcacgggcgtgcctctgcccagagcttctcagccgtcagcccacatcagcccacgccaacggcgagccatcactgtggaggccctctgtgagaaccacttaggcccagcaccaccctacagcatttccaacttctccatccacttgctctgccagcacaccaagcctgccactccacagccccatcccagcaccactgccatctgccagacagctgtgtggtatgcagtgtcctgggcaccaggtgcccaaggctggctacaggcctgccacgaccagtttcctgatgagtttttggatgcgatctgcagtaacctctccttttcagccctgtctggctccaaccgccgcctggtgaagcggctctgtgctggcctgctcccaccccctaccagctgccctgaaggcctgccccctgttcccctcaccccagacatcttttggggctgcttcttggagaatgagactctgtgggctgagcgactgtgtggggaggcaagtctacaggctgtgccccccagcaaccaggcttgggtccagcatgtgtgccagggccccaccccagatgtcactgcctccccaccatgccacattggaccctgtggggaacgctgcccggatgggggcagcttcctggtgatggtctgtgccaatgacaccatgtatgaggtcctggtgcccttctggccttggctagcaggccaatgcaggataagtcgtgggggcaatgacacttgcttcctagaagggctgctgggcccccttctgccctctctgccaccactgggaccatccccactctgtctgacccctggccccttcctccttggcatgctatcccagttgccacgctgtcagtcctctgtcccagctcttgctcaccccacacgcctacactatctcctccgcctgctgaccttcctcttgggtccaggggctgggggcgctgaggcccaggggatgctgggtcgggccctactgctctccagtctcccagacaactgctccttctgggatgcctttcgcccagagggccggcgcagtgtgctacggacgattggggaatacctggaacaagatgaggagcagccaaccccatcaggctttgaacccactgtcaaccccagctctggtataagcaagatggagctgctggcctgctttagtgtgagtgctctgccagagggaaagctcctagaacagtgagaaggccctccaggggaattcctcgaatactcagaggcagtagtgtggggtagtagttgaagcacacagctctagagtcagacaggcttggattcatatcttggttctgtgaccagccttgaatgagttatttaacttctctgagcaatatttttctcgtctcatttataaactagggatgataatggtatatgagataatacatgctgtgggcttagcacagtgcatgatacacaaacatgcaataaatattaccttgttattcttttgggctctttgactctctcactttctgcaccagaaagaaaaaggatcaagttagaggactctaaatttttcccctagagagtgagaattggaggctggcagaatacaggaagataaggtaggaatgagaaagattcagggacactaccaatcagaagactttggttctaggttcaactgtgccacaaattagtgtgatcttaggcaagcaatttcatttagtttttctgggcttcagtttttagtctgtagaatggaggggtgagaatatgttaaacaccataattaattcactgagtgcctattatatgcaaggcactttgctaggttctgtaggatatataaagatttcttactccatgttggggccacctttttcaaaccctgggcccagtaaaatggaattagatagtctcatagtatttggttcaggtctacaagtattaattgagccaactatggacctggcatgggagagggtacaagagaaattagagatatgatcccggacctaaaagagcttaatatctgaagaatcacacttgagatgatggacaagcatcccagcaagtggagctggaatgcctgggggagctgcaggagagacagagaagacagctctgttggcatattgtctttcttcccaccagcctgtgctgtgggatctgctccagagggaaaagagtgtttgggccctgcagattctagtgcaggtaacaggtggagggcacatgggtgggctgggtgacagccatggctggaggtccctgccccgtgaggtgaggccatacccaccatgacctcctattcgcaggcgtacctgcatatgcccccagaaaacctccagcagctggtgctttcagcagagagggaggctgcacagggcttcctgacactcatgctgcaggggaagctgcaggggaagctgcaggtgagcactgagaaaggggagcaagggcacctggagcctagtgttcagagggcttgctttagtgggaggaggaactccagagaggaaatggcagggatactgagcatctccagaggcagaatccattcctgtgcccctacaggtaccaccatccgaggagcaggccctgggtcgcctgacagccctgctgctccagcggtacccacgcctcacctcccagctcttcattgacctgtcaccactcatccctttcttggctgtctctgacctgatgcgcttcccaccatccctgttagccaacgacagtgtgtaaggttcttgcactactcctcctgctcctgtcacggtcaggccaaccgcatccacctggagcagccccttccggagctcctctctgtttttttctttcatgccagataggcaatgtgccaacatcgtagcaaggtttgagagaggcacatctcacgcctgagtgtgaaaacccaatcattatgctaatgaactacaaaaggatcagagagctcctctctattaaaaccagggagaggatgggcgtggtggctcatgcctgtaatcccagcacgttgggagcccgaggcaggtggatcactaggtccgcctagtgagttcgagaccagcctggccaatatggtgaaaccccgtctctattaaactacaaaaattagccaggcatggtggtgggcgcttgtagtcccagttactctggaggctgaggcaggaggatagcttgaacctgggaggcagaggttgcagtgaaccaagatcgtgccactgcactccagcctgggtgacagagcgagactccgtcttaaaaaaaacaaaaaacaaaacaaaacaaaaaaacagggagagtctccttcctatctagacagcagggctacagagggtcagaggaaaacagtttggaggaagacaaagggttaagacccatgactcctcgcagcctggctgccatccgggattacagcccaggaatgaggcctgaacagaaggaggctctggcaaagcgactgctggcccctgaactgtttggggaagtgcctgcctggccccaggagctgctgtgggcagtgctgcccctgctcccccacctccctctggagaactttttgcagctcagccctcaccaggtatgagaatcatcttctttacttgactggcccatcttctgctagtggggacaaagagtcaatggcatgtctctcagtggcccctccctgcaagaaccctatagtgaccccagtgcgagctaaccttccccatctcagatccaggccctggaggatagctggccagcagcaggtctggggccagggcatgcccgccatgtgctgcgcagcctggtaaaccagagtgtccaggatggtgaggagcaggtacgcaggtgagttgttgtgggatcagtaaccaaggcaagagtggaagaggtagagagaggaaggcacagctgtcacgctgggtcggtgttctaggaagaaaggggcaagagagtaggcagtggcctcaggcagcatagagttccaggagagaggtctatagatggtgcccctgtgtagtggtgtagtgtcagagtgcccagtgtatgtacccataccatctgctgccaggcctgccttagtgctagtcttggggaccacacaaaggtcagcttcatgccctcctcaggcttgggcccctcgcctgtttcctgagccctgaggagctgcagagcctagtgcccctgagtgatccaacggggccagtagaacgggggctgctggaatgtgcagccaatgggaccctcagcccagaaggacgggtgagcccctcagcacaagcctacaagactttaggcttcccctgggtctgtgtggatggctttcccattgtgtcaacttgagcacagtggtgccagcccccatcccacttttgcaacctccattccttactccatggccattcttacctgttaccacctcttcctggcccttctctatctggtctgtagcaccccaaacataccctttgccattttgaacctaatctactccagtccaatccctagttccaaaccctagcccaggccctgggaaattcagatgtgggattagagaggaagttcaaggttcatctgtcttttctctccagtcctaaaccttctttggttacaggtggcatatgaacttctgggtgtgttgcgctcatctggaggagcggtgctgagcccccgggagctgcgggtctgggcccctctcttctctcagctgggcctccgcttccttcaggagctgtcagagccccagcttagagccatgcttcctgtcctgcagggaactagtgttacacctgctcaggtttgcctgtctcactccctggcatgtaccctccatccccgcttgagccccagtcaagagaatcccattcagggataaaagcagcccctcctttccctgggtgaacagtagaggtaaactctgtctgcaggaggacgccttcattccctttcctcagatcaagaagggacctgagtcactgaggatggttactagggatggttaagaggcagcgggaagttttggagggtttgccttaggaacccacttaggacctggctgctgggtcctgagagctgttgttttcggtcccatcccaacacaggctgtcctgctgcttggacggctccttcctaggcacgatgtgagtagcagcaacttctcagcctcccgccagaggtctctatcctcttttaacctggctcctgcatctgcccctcctctctctccgctcccctcatacttactgccttgctgcattgtgattgttgtcttccccaacacccttcccttcttcttcaggcctcttgtctctcttgctctttagctatccctggaggaactctgctccttgcaccttctgctaccaggcctcagcccccagacactccaggccatccctaggcgagtcctggtcggggcttgttcctgcctggcccctgaactgtcacgcctctcagcctgccagaccgcagcactgctgcagacctttcgggtatgagagtggcaaggaggatgagataatcagggataccggctctttctggttgggaggaaggcatcttccctgaggccagggaaggcctttcatacctccccacttacacacacacacacacacacacacacacacacacacacaaccaattctcatgcaggttaaagatggtgttaaaaatatgggtacaacaggtgctggtccagctgtgtgtatccctggtcaggtaagtgtgagatctcccaactgagctcctctccccattctggggcagtttcatatggctggtgctacctcccacactaccctgcagtggccctgagagttctggttagctctgtgcccattagcagccctccccagtgccagatgcaggacagcatgatccactcacattgtcctagactaatgtcaaagctggaagggcctgagaaatcttccaggccacccaccctgctttcagatgaaaagaccaaggctgggagaagctaagggactttgtttgcctggtgcctaactagcagcaacacttgaccacagcagcctgcagtgtgaggctcttaggcgtttattgctacagtggcaaatgccattccacttctgtcctagctttggtccctttccacccccatggttccttttctctgagtgctaagtacagactctctcacctatcactacactgctatacccatcaccgccagcagcctattcccaccacctggccagactgcctgcttcccctgctcccattaaagctgctacaactggattccttggctcttctggcaaatcgaagacgctactgggagctgccctggtctgagcagcaggtaattctccccacttaatttcagaacttcctccctcaatgtagtctaccttctttacctatcccttagccctatttggccagcttatccctactatcctttatttgattgtttgagatacagtctcactctgttgcccaggctgcagtgcagtggcatgatcagagttcgctgtaacctcaaactcctgagctcaggcaatctttctgcctcagcctcctgaatagctaggacgacaggtggttaccaccatgcctggctaatttttaaattttttttttgttttttgagatgaagtcttgctctgtcacccaggcttgagtacagtggcacaagcttggctcactgcaacctctgtctcccgggttcaagcgattctcctgcctcagcctcccgagtagctgggactacaggcactccccacaatgcctggctaattttttttttgttttagtagagacagggtttcaccatattggccaggctggtctcgaactgctgaccttgtgatctgcctgcctctgcctctcaaagtgctgggattacaggtgtgagccaccatgcccggccaatttttaaattttttgtagagacagacaatacaaaaatgtggacactatgtggagacactatgttgaggtactatgctgtccagattggtcttgaactcctggcctcaagcaatcctcctgccttggcctcccaaagtgctgggattacagacctgagccactgcacccagccccctagtatctcttataatgtgacttgcttttctttttctttctccttcccttttctttcatttctttctcactctcgagagaagagtgggcatctgggagagtgggaggctggtgggtcccacagagtgaggaggcaggactgggtccaaggcagtcctgcctctccactctagggggtatccttggacagtgtctcttctgggaaggggctcgtctttctttctcttgtaggcacagtttctctggaagaagatgcaagtacccaccaaccttaccctcaggaatctgcagtgagtaacttgtgttgagcagtgcgctgaattcgaccaacatttttttgagtgcttactatgtgccaggcaccatgtgatatggaatgggggatatagggatgaatgatgcatagtccctgcctcgtggacgttctcctagcacctccctttgccctcctttccttccacagtgccatgcctatcctgactagagccaaaggactcagaaaacctggattcaggttccagtcctgtcacctacttgtcctcttgggcaagtcatttaacgtccctgtgtcagttttcccttctttaaatgagaattacaatggcaccagcctcataggtagttactgtgaagattaaatgaggtaggtcatgtaagatatttaacacagtgtttggtccattgtaaagtcccagtagtcatttgctactgttagtttacttcaggatgacttcagaggcactggccaagcaagaataaataggaataagaaggtatcactttacttacacccacattagaagaacaatgggcttcagaatctttttttttttttttttttcgagacagtcttgctctgttgcccaggctggagtgcagtggcgcgatttcggctcactgcaacctctgcctcccaggttcaagcgattctcctgtctcagcctctggagtagctgggattacaggaatgtgccaccatacccagctaatttttgtatttttagtagagatggggtttcaccattttggccaggctggtctcaaactcctgacctcaggtgatccacccgcctcagcctcccaagggcttcagaatctaagacatggctctagtttcagtttaccacatttctagcagaatgatgttgggaatgtcacctgacttccataaatccttattttctcctctgataaacagcagtgatgttatggggagctgatgagatatctatgtaaaaacatttctcaaaccataaattacggtggatgaacatctgtacttgtgttgagagtactgatatcaaggagcaaacaggctgttgtatgtgttgaatgagcctctccccactcacacacccacagggctctgggcaccctggcaggaggcatgtcctgtgagtttctgcagcagatcaactccatggtagacttccttgaagtggtgcacatgatctatcagctgcccactagagttcgagggagcctggtgagagggggtgcctggactttagtgggagcagggaggctgggaccctaggtatagaacccagctcctatgttctgctctggcctcacactgcttccctacagagggcctgtatctgggcagagctacagcggaggatggcaatgccagaaccagaatggacaactgtagggccagaactgaacgggctggatagcaagctactcctggacttaccgtaagtactgcagctagagatattggcccctcagaaagctcaatctggggtgaagatctgcccttagggaatgccctggaggaggtagtttttctgtctggtagttccctgacataatttatagcccaaagcagaggattttattcaaagttgctctatgtattgactggttcccagaatatgctccagcacagggcagctgagggtggcaacactgtattgaagcctgccaagtaatcttacaataacctagtccacattaattgagattgagacagagcatctgaagtgagggaggcaatgctccaaatctgccccagaggattgtagtttgctcagggcactgtgttcttagtgcattcagaggagtagatcgagagaaaaatatatgaaaaatgtgataaataccttcaaatacctgaggggctatcaagtagaaattagattgtcatatttatgagtggccccattgggcaagactaagagtagttaacggagatcagatttttacatagtataagaaaaactaaggtagtgagttcctggtccttggagctgttcgagcctaagccagatggccccatggcaggaatgttgtagagcacgttcatatacaggttgtgggaagaaaaggctataggaacccaaggctcctccctacccatggagaaatttattagtatgttactcatatgctgcttttctcattttacccctaccaccaccccgttgccatccgcactgtaagtcaggataggaaaatgctggtgttacagtcttcctggggaatatggagctgaagtggagtaaaagcagttgacttcattcctacttttttcttttttttctttttttttttttttgagacagagttttgctgtgtcaccaaggctggagtgcagtgacgtgatctcggctcactgcaacctccatcttccaggttcaagcaattctcctgcctcagtctcccgagtagctgggactgtaggtgtgcaccaccatgccaggctaatttttgtatttgttgtagggacgagctttcaccatgttggccaggctggtcttgaactcctggcttcaagtgatctgcccacctcggcttcccaacattcttatatttttataggcctttccacagatttcagctcttgtatgacttagcccagttccagaactggtaatcctaggtagggtacaggttatcacctctgatttcgggtaaaagggatttatttatttatttgtttatttatttatatttttgagacagagtctcgctctgtcacccaggctggagtgcaatggtgccatctcggctcactgcaacctctccctctggggttcaagcaattctcctgcctcagcctgctgagtagctgggattacaggcgcgtgccaccacacccggctaatttttgcatttttagtagagacggggtttcaccatgttgctcagggtggtctcgaatttctgaccctgtgatctgcctgcctcggcctcccaaagtgctgggattacaggcatgagccactgcgtccggcctgtttttacttttttttaatgccattcagatctgtttaaatatgtgggttctgtgagataatttagaatcccaaggttacagatgaggtgaaagatcctagaccatgcatcaaaaaacttgagtttctcatttgtgaaagaaggataagagaaacacctattttgtctgggtgcagtggctcatgcctataatcccagcatttggggaggccaaggtgggtggatcacggaggtcaggtgttcaagaccagactggccaacatggcaaaacaccatctctactaaaaatacaaaagttagctgggcgtggtggcacgtgcgtgtaattccagctattcgggaggctgaggcacgagaattgcttgaacctgggaggtgcgggttgcagtgaactgagatcgcagcaccactgtgctccagcctgagtgatggagtgaggccaggtcttgttgtaggatcaaatgagataacacctgaaagaactttgtaaattgtatagcacgtacaaacaagaagggacctcttcacaagcagaggaagggtggtcctgtggaaaaaaacgggaattgggagtgagagacctcaacatttgatctctgtgaacctcagttttttaatctataaaatggggaaatgttaatggtacttaatatttggagcttttgagtccattagatcaggtaggattgttcgttattttttttttttaggaagactagaaatatgttgctccctttttctcccccactcaagcttgatggtgggaattggccctggagctgtttactatcagttcctgtccagcttcactaaatttggtctggggtcacatcttagctgcggactgtggggttttgtggtcccttctcgacttggcccagctccacctgaatcctgttgttgtcaaattgctgtaataggatccagttgatggacagactatccaatgaatccattatgttggtggtggagctggtgcaaagagctccagagcagctgctggcactgacccccctccaccaggcagccctggcagagagggcactacaaaacctggtaagagtccaccctaccagactcagatttgctgccctgggcaattcttgctcctcagacaatgctctctgactgtcccccaaccctctacttcttgctttcttgctgccaaacagattcctgtctacaaggcctggcccctgttttgcctctgggttctgttccttgataatatgcttcacgttacttgtccatacctcttggagtccgagaaatctcttggagtccacctctcagtctttctgcctgctcctatctgggctcattgcttaaggaagtgaacaaaggtagtgagcatcatagggtgctgagctgggagcaggagggagggaaggttagggggcttggtgtcttgatcaaggtgtctggtattctgagtcagaagtgcattgtccaagttctgatgctcttctccaggctccaaaggagactccagtctcaggggaagtgctggagaccttaggccctttggttggattcctggggacagagagcacacgacagatccccctacagatcctgctgtcccatctcagtcagctgcaaggcttctgcctaggagagacatttgccacagagctgggatggctgctattgcaggagtctgttcttgggtatggaccttcgagaacttcagattctaactcattctatacccagtccctcagccaccatcatcagtggcagcctgttccatattcttaaggtcccctggagccctgtgtccgaaatcctagcatgtcctcttttccccttccttttcctcacagttccctcagctccccagcccccgattttcttcctgtccccaggaaaccagagttgtggagccaggatgaagtagagcaagctggacgcctagtattcactctgtctactgaggcaatttccttgatccccagggtgagatgaaggaagaagggaagggagtaaatgcatagaggggactggtgagctggttatggggacccgtggccaaagagggcaaaggatatgaagcctagatctggggggagactgcaaaacagagacaggactttggacttagagctatagcagcaggtcctgatctgtccagatctccccactctccttctaccttctcatgcaggaggccttgggtccagagaccctggagcggcttctagaaaagcagcagagctgggagcagagcagagttggacagctgtgtagggagccacagcttgctgccaagaaagcagccctggtagcaggggtggtgcgaccagctgctgaggatcttccaggtgaaactacccaaatacttatatgtccagcaggatgtacagggagtatcaaacggtctgggttctacatgtgctcttccctgggactgggttttctaatttataaagcaaagagtttagagggatgatcttcaagcctcttgtagttctagaattctgtagttctgggagtttgtaaactattaagttttcttttagcccagaacttccattttcctgctctctcgtgtctgctctagactcagctctagctcggctaagtgtggagctctctgctggggagatccctagaagctttgaaggagacattgtgaggctggagaactgggttcaaattcagtgctaccattaaatctctgaataacatcctcagtcttccatctataaaagtcttggcatctccaatcacttcttgttctattatctcctaagccctatacatattactctgtaatactcctttgatccctatttctcacagtgctctatcctccaaaggttggaagactcactctatctacagatatctctctgggcatattttattactgcgctgacctcctggccctgccttcccccttcagaacctgtgccaaattgtgcagatgtacgagggacattcccagcagcctggtctgcaacccagattgcagagatggagctctcagactttgaggactgcctgacattatttgcaggagacccaggacttgggcctgaggaactgcgggcagccatgggcaaagcaaaacaggttagggatggagagccaactggggttggccatgaggaagctatttgggtgtgatgtaggacacaaagagaatggagagttggatgagaggtgggggaagcaagagatagaagagttagaagatttgggtcacaagtaggaggtgaagggagataaatattgaggaaagagagctagtataatgaatagagggacgaaagcagtggttaccaaattttaatgcatatcacgatcatcaagggaacagatttttttctttatttttttttctttcttaaaaaaataatggcatgcttcggctgggtgcagcggctcacgcctataatctcagaactttgggaggccaaggcgggcagatcacgaggtcaggagatcaagaccatcctgtctaacacggcgaaacacggtctctactaaaaatacaaaaaagttagccgggcatggtggtgcacacttgttgtcccagctacttgggaggctgaggcaggagaatggcgtgaacctgggagggggagcttgcagtgagccgaagtcaagccaatgcactccatcctgggtgacagagcaagactccatctcaaaaaaaaaaaaaaaaaaaaaaggcatgcttcatgaatttgcgtgttatccttgcacaggcgccatgcaaatctctgtatcattccaattttttggggtatgtgctgctgaactgagcatgggaacagtgccagtgccagattaccatgcttcactgacttaataaaaacctttggggaggctgggcgcagtgactcatgcctgtaatcacagcactttgggaggcggaggcaggtggattgcttgagcccaggagttagagaccagactgggcaacatggtgaaaccctgtctctactaaaaatagaaaaaacattagctgggtgtggcggcacatgcctgtaatcccagctactcaggaggctggggtaggagaatcccatgagtgcaggaggtggagggtgcaatgtgccaagatcgcaccactgccctccagcctgggtgtcagagcaagaccctgtctcataaattaaaaaataagcctctgggggaaagagtctagacatctgcatctcctttttttttttttttttttttttttgagacagagtctcactctgtcacccagcatccaggctggagtgcagtggtgtgatcttggctcactgtaacctctacatcctgggttcaaacgatcctcctgcctcagcctctcaagtagctgggactacaggtgcaccacacctggctaatttttgtatctttggtagagatggggtttcactatgttgcccaggatggtctcgaacttctgggctcaagcaatcctcccacctcagcctcccaaagtgctgggattacagctgttagccactgtgctgggccctaggcatctgttttaataagcgtctctgtgtctgatgcacataaaagtgtggaactcatggactagagttagtttgctcttcttttccactgattgtaatgtctttcaaaacaccttagaggaactgtaaggcaacggtctcattttatagtggaggaaactaaagaaaaggcaaatgatttacctagagttatacagctaagggcagaggcaagacttaaaacccagcagtatgactcccaatccactgcttttccactcacattgttcctgtctttctcctagttgtggggtcccccccggggatttcgtcctgagcagatcctgcagcttggtaggctcttaataggtctaggagatcgggaactacaggagctgatcctagtggactggggagtgctgagcaccctggggcagatagatggctggagcaccactcaggtaacacttttcctcctccctacggcttcccaaacacccatcccacagacccagccctatagatcatctaaagcccaaggaatttttttcctgtgaccctacctggtccttctttctatcttttgttgataccccatactagtgaccttcaggactctgatttattcactctgaggccctggacacataatactgtctcctacctcttttcctggaggcttcctctttttctttccttttcttttctgagtcctcagccttccccatgactccttaggtcttaatagtaacagaatataacccagtaacacctatcacttccctgtccattaattctccataactttcctccttcccctcttctcccaccccccaccccagctccgcattgtggtctccagtttcctacggcagagtggtcggcatgtgagccacctggacttcgttcatctgacagcgctgggttatactctctgtggactgcggccagaggagctccagcacatcagcagttgggagttcaggtcatttgtgaaggggctgagggtggtggtgctgaggtaaaggtggacttactggggaaagaaggatcatgaaggtctggtcccatggaggaagggaactcatttgaagccatctcttcctttgtctcatgaccacagcccctttcactgaagccgaattcttcttccttccttcctactgttctacagccaagcagctctcttcctcggcaccctgcatctccagtgctctgaggaacaactggaggttctggcccacctacttgtactgcctggtgggtttggcccaatcagtaactgggggcctgagatcttcactgaaattggcaccatagcaggtggggagctgggccactgctggtgcaagttggtttggtttctataccatgggtggactggatggaagactgccctgcaattcttaaggtgggggcctgagggtgtttaaataaggggctagagacatattggggaaggtctatgatagggcactttgggagtagttagagaaggtctataggtttgaagagagggaaggtcagtctaagacaatgtttggatgccacttgcttcaacagctgggatcccagacctggctctttcagcactgctgcggggacagatccagggcgttactcctcttgccatttctgtcatccctcctcctaaatttgctgtaagtattaatggactggggtgaccacaggagagccagggcccaatggggactacatgcatgcactgattcctacccctgccctcaggtggtgtttagtcccatccaactatctagtctcaccagtgctcaggctgtggctgtcactcctgagcaaatggcctttctgagtcctgagcagcgacgagcagttgcatgggcccaacatgagggaaaggagagcccagaacagcaaggtgagttcccagctgcacagcttgatcctccatctcctgacccagaatcaaacccctaatttggtgctgtctggctcttagagtgcacccagggagatccctggagtgaaggagtctacaggcagagcgctaatttccaagtatcaatgctcctggagagctgagttgtgatattactcccattccctgtctattataggtcgaagtacagcctggggcctccaggactggtcacgaccttcctggtccctggtattgactatcagcttccttggccacctgctatgagcctgtctctacagtagaaggagattgtggggagagaaatcttaagtcataatgaataaagtgcaaacagaagtgcatcctgattattttcagaagctgatgaggaata

SEQ ID NO: 4 - DNA, 2301 bp, human
ccaggcaggtaaggatcaggggtgataggggagagcaattactttgttcactgtatgcacccgcgggggcctgggagtccccatttgcaggtgggtagggcctccagcccacaccacccagacctaggcttccctcttctcaggatccaccacagggttaggggacaggaagcctgttctattctcaataaatcttacaaaattccaaaaagactttcctgtgtccctctgtccccctggggggaagggacaatggaggaatattccccaggcctgggcgactgtccgctggtcagagggaaggccccgctgcctgcccggtgcacctgtgagctgagattgtggggatcattcagtcattcctttattcagtagatctgtacggggctggctttgtgccaaggcctgccccgggcacctgggatgtggggaaccaggcatagcaatccctgctctgggacagtctacaaacgagcagcgagaacaaagacatactctggtcatgacagacactaccccgcacattactgatggggcagtgtgggcctgctgggttagagacggcctactccctgatctgtgcatgaagggctgggaagaggagatgggccttcggggcacaggaaggtgtggggggaccagagcggaggctgctgagggcagatgcaggcatctagggccaggaagctggtgtggtggtggggagaagcagggttgggatgactgaaagcagagtggacgactcaatgaaaaacgacttgggagacggggtgagtgcagtgtgagggcaaagtgaaccgagatgcctcgctttcttcatgggactgagaaatgggtattgcaaatcgtgtgatctgcttagcatagtgcctggttcatagcatggctgaacacaatgtagctgtcatcctggtgtttggaggccgccctgtcccccataacctgcccacctctctgccccattcccacagctcactgcattttgtccctgctgccagtcgtgtgccatgggctctcctgtgctcccccggggtctcatttccccatccgtgtagggctgacgccactggccccgcccagcctgggaggatggagaggggggacttgaggagtggccagtactgggtttcggcagcaggtgcaaatcccaggtggggactgtgttccagggactcctggactcctgttaggccagcaggctaaggggcagacggcatcctggggtcttcctgggacagggtcgtcgggtctgaggggacccaggccctagcagaagctgactcctgtcacggtcacttcccagcggtgctcagcccgcctgacggaagcagctgggcagtggggcctgtgaccagcaggcggcgctggcgagctatagcgcccctttgggcccccaccccaccttctgggcagcttccctgcagacaccccagttatgggggctagggacccaaaagagacatccttctgccacccagagctgccctggcgaggtgcactatggggccgccgacagctgcgtggctgccgagggcggaaaggagaaactgtcatgtcccgatagggccgcgcgaggtctccatcctcgacaacgctaataacaaagacgtgtgctcctctttgcttggttccccccactcctttaaatcacagatttcacttcagtttatctgtgtcgctgtcacacgtggggtggctcccagtcagctggtttggcaaagtttctggatgattacggaataacatgtgtccccaacccgcagagcaggttgtgggggcaatgttgcattgaccagcgtcagagaacacacatcagaggcaagggtgggtgtgcaggagggagaaggcgcagaaggcagggctttagctcagcactctccctcctgccatgctctgcctgaccgttccctctctgagtcccaaacagccaggtagaggaggaagaaatggggctgagaccccagcacatcagtgattaagtcaggatcaggtgcggtttcctgctcaggtgctgagacagcaggcggtgtcctgcaaacaacaggaggcacctgaagctagcctggggggcccacgcccaggtgcggtgcattcagcagcacagccagagacagaccccaatgaccccgcctccctgtcggcagccagtgctctgcacagagccctgagcagcctctggacattagtcccagccccagcacggcccgtcccccacgctgatgtcaccgcacccagaccttggaggccccctccggctccgcctcctgggagaaggctctggagtgaggaggggagggcagc

SEQ ID NO: 5 - DNA, 2343 bp, human
agtgctggctggacagctgctctgggcaggagagagagggagagacaagagacacacacagagagacggcgaggaagggaaagacccagagggacgcctagaacgagacttggagccagacagaggaagaggggacgtgtgtttgcagactggctgggcccgtgacccagcttcctgagtcctccgtgcaggtggcagctgtaccaggctggcaggtcactgagagtgggcagctgggccccaggtaaggatgggctgcccactgtcctgggcattgggaggggtttggatgtggaggagtcatggacttgagctacctctagagcctctgccccacagccacttgctcctgggactgggcttcctgccacccttgagggctcagccaccacagccactgaatgaaactgtcccgagcctgggaagatggatgtgtgtcccctggaggagggaagagccaaggagcatgttgtccatcgaatcttctctgagctggggctggggttagtggcatcctggggccaggggaatagacatgctgtggtggcagagagaagagtccgttctctctgtctcctttgctttctctctgacactctttatctccgtttttggataagtcacttccttcctctatgccccaaatatcccatctgtgaaatgggagtatgaagccccaacagccagggttgtagtggggaagaggtaaaatcaggtatagacatagaaatacaaatacagtctatgccccctgttgtcagttggaaaagaaattaacttgaaggtggtctagttctcatttttagaaatgaaatgtctgtctggtcattttaaaatgtggcccttaaatttcacgccctcaccactctcccccatcccttggagccccatgtctctagtgaaagcactggctctgcccccagccctcatggctcatgctggcatagggcgcctgctccacagcctgggcaccatcttcagacaagtgcccggtggcaactgcctgctggccctgttgaatccacatctccaccaggcatccagactagttcaggtctctggaaggactgtgggtttgctgtgtcccagagctccagggcaggggtcagggctcggatgtcgggcagtgtcatgggcagaggatcgaatgccccggcggctctgaatgggcccttgtgaaaaattgatgcgcattctaggagacaggttgggagccagaggggcctcataccagggtctgtaggctggggctgccttttaagctccttcctgaggccgtctctgggtctggccctgtgctggacaaggctggagacaaggcaatgtctcagaccctctcccattggccacatcctgccctggatcaactcgccaactttgggggcagaggtgggactgacccttaccctgacaacataatgcatatagtcaaaatgggataaaggggaatatagaggctcttggcagcttgggagtggtcagggaaggcttcctggaggaggtatcatctgaactgagccatgaaccataagtggaaattcactagtcaaaatttcaggtagaagggccagtgtgtgaaggccaggagatggcaagagctggcgtatttcaggaacagtgagtcactgaggatgtccaagtataagggtaggaaagggagtgagcagtgagagaaaagaccgaggcatcagcaggggccagattgtgctgggcctagcggggcgggcccgggcccgggcccaggcccaggtgcggtgcattcagcagcacagccagagacagaccccaatgaccctgcctccccgtcagcagccagtgctctgcacagagccatcctgagggcagtgggtgctcttgagaggtttcaggcagggtgtgctgtgagcaggtcatgcccagcccttgaccttctgctcagtcaggcttgtccttgtcacccacattcctggggcagtccctaagctgagtgccggagattaagtcctagtcctaaatttgctctggctagctgtgtgaccctgggcaagtcttggtccctctctgggcccctttgccgtaggtccctggtggggccagacttgctactttctaggagccctttgggaatctctgaatgacagtggctgagagaagaattcagctgctctgggcagtggtgctggtgacagtggctgaggctcaggtcacacaggctgggcagtggtcagagggagagaagccaaggagggttcccttgagggaggaggagctggggctttgggaggagcccaggtgaccccagccaggctcaaggcttccagggctggcctgcccagaagcatgacatggtctctctccctgcag

SEQ ID NO: 6 - DNA, 2063 bp, mouse
agagggatctgtctgtttcatttttcccgtaccccgcacccccccccacacacacacacacaagggtaaagacagagggcacaatgggtagctgacctctggtcagaaggatgagcaaggaaggccttgggaatcacagataaaagctggccttgctggttacctagtgagtcagagctcagctctgtggaatccttcaggttccctgtcagtgtggggctggctttgccgagctctgcccatctcaggccctggggacatgggggcacacagttcctgctctgcagcagccttccagcaactgaggaaaatgtacaaatcatgtctgatcgaagtactgcagtgtccttgataggcagtgtccctaggagctgactctcagtctctgagttcaagatctgtgccctgttctagtggagaggagagacttacatgcaggccacagacagaggtggcagagcagagatactgggaaggtgcggtagctagagctaggaatttatggccagggccacgggcagaagctgggttgggttgtggaaaacgaaatgatggaatacgacgatagattggatttggaggggcgagggtggtatgaacttaagaccagttctgcagtggttcagatgtggtgcccttaagaggctcaggtgccagaaaccagtgccccttctggatagggcagaaaacaggcagaacctggagaggtacggtggagcatgaggtggtaaggacacacaccatctcaggtggtttgttttgggggaaacaagcatggatttctaggtttctccgatcgggtgacctgcttagccgagtttgggtgctgaggaaatgtctccgttatggttctgagaccactttctcacccacccactgccttcccagcatgcacctcagccccaacgaagcacacctgccccagttgccgccttgtcgattgggctctggatgctccaacctgtgtctctcctactctgtgtaggctcatgctactcacctgactcataagtatcagtttcgtaggtgaaggctgcgttgggtgcaagtcaaccttggaccgtggtctcactgtggcaccctggtatgaggaagttgaccggcttttccttagtcttgtagcagttggctgtgcccagcaggtggcaccattgcaccattgattctccccactccatacccactatagattccaccccaccctattcccctccccaccttaacccacccacccccaccgaaaagcagctttcctgagtagatgtcccagctggcaagtgtgggcagaagaaggggccaggtctcaggaggaggaggaggaggaagaggaagaggagtcctccatcctccatcctcatctaccatgatgagtatttgtgtcctgttcattcccacccactcccttttttaaatcacacatttaaatcacacacacacacacacacacacacacacacacacacacacacacacacacacacacacacacacaccgggtaagtctgtcctgcgtgaggtggctcctactcaggtggctttgcaaactgtctggataacagcacactcaagactcctaggacaggctgtgggggccagttgtagagcttgggggtggggtgcatcttggggagtcctggtttggatgttgtgtccagccaaggctccaggtattgcaaagcctgctcatctatgtggtctctagtagtgccctgacgagggaagctgggtgagcaggggaggctactgggaactgagacccagcaaaatcatgaggaagatgggacgtgatcaggtgtcctaaccatgcagagatggcaggtagtaacacatgtgacaagagaccctgaggtcctgatggttggccccaggcccgaggttcccactggccagcagtgccccctggagcttctatgccttgcatccctgctggttagctttacacagcaccttgggcaacctctagacgttagtcagcagccccagcacagcccgcccctcatgctgatgtcaccacatccagaccttcgaggcccccagggctccgcctcctgggagaaggcttt